(19) United States
(12) Patent Application Publication
(10) Pub. No.: US 2022/0339127 A1
(43) Pub. Date: Oct. 27, 2022

(54) DRUGGABLE TARGET TO TREAT RETINAL DEGENERATION

(71) Applicant: The United States of America,as represented by the Secretary,Department of Health and Human Services, Bethesda, MD (US)

(72) Inventors: Kapil Bharti, Bethesda, MD (US); Karla Yadira Barbosa Sabanero, Bethesda, MD (US); Justin Ren Yuan Chang, Bethesda, MD (US); Balendu Shekhar Jha, Bethesda, MD (US); Ruchi Sharma, Bethesda, MD (US)

(73) Assignee: The United States of America,as represented by the Secretary,Department of Health and Human Services, Bethesda, MD (US)

(21) Appl. No.: 17/642,610

(22) PCT Filed: Sep. 11, 2020
(86) PCT No.: PCT/US2020/050540

§ 371 Date: Mar. 11, 2022

Related U.S. Application Data
(60) Provisional application No. 62/899,899, filed on Sep. 13, 2019.

(51) Int. Cl. A61K 31/155; A61P 27/02; A61K 9/00

(52) U.S. Cl. CPC A61K 31/155; A61P 27/02; A61K 9/0048

(57) ABSTRACT

This invention relates to novel method of treating or ameliorating a retinal disease or disorder or retinal degradation in a subject and a novel method of restoring retinal pigment epithelium cell compromising the administration of a one or more compounds which modulate Nox4, formation of radical oxygen species, serine protease, a dopamine receptor, NF-kB, mTOR, AMPK, RPE epithelial to mesenchymal transition, RPE dedifferentiation, or one or more Rho GTPases; and kits for administration of the methods.

### CROSS-REFERENCE TO RELATED APPLICATIONS

This application claims the benefit of U.S. Provisional Patent Application No. 62/899,899 filed on Sep. 13, 2019. The entire contents of this patent application is incorporated herein by reference in its entire

### STATEMENT REGARDING FEDERALLY SPONSORED RESEARCH OR DEVELOPMENT

This invention was made with Government support under project numbers Z01 #: EY000532 by the National Institutes of Health, National Eye Institute. The government has certain rights in the invention.

### BACKGROUND OF THE INVENTION

The retina is a layer of specialized light sensitive neural tissue located at the inner surface of the eye of vertebrates. Light reaching the retina after passing the cornea, the lens and the vitreous humor is transformed into chemical and electrical events that trigger nerve impulses. The cells that are responsible for transduction, the process for converting light into these biological processes are specialized neurons called photoreceptor cells.

The retinal pigment epithelium (RPE) is a polarized monolayer of densely packed hexagonal cells in the mammalian eye that separates the neural retina from the choroid. The cells in the RPE contain pigment granules and perform a crucial role in retinal physiology by forming a blood-retinal barrier and closely interacting with photoreceptors to maintain visual function by absorbing the light energy focused by the lens on the retina. These cells also transport ions, water, and metabolic end products from the subretinal space to the blood and take up nutrients such as glucose, retinol, and fatty acids from the blood and deliver these nutrients to photoreceptors.

RPE cells are also part of the visual cycle of retinal: Since photoreceptors are unable to reisomerize all-trans-retinal, which is formed after photon absorption, back into 11-cis-retinal, retinal is transported to the RPE where it is reisomerized to 11-cis-retinal and transported back to the photoreceptors.

RPE plays an important role in photoreceptor maintenance, and regulation of angiogenesis, various RPE malfunctions in vivo are associated with vision-altering ailments, such as retinitis pigmentosa, RPE detachment, displasia, athrophy, retinopathy, macular dystrophy or degeneration, including age-related macular degeneration, which can result in photoreceptor damage and blindness.

General retinal diseases that can secondarily effect the macula include retinal detachment, pathologic myopia, retinitis pigmentosa, diabetic retinopathy, CMV retinitis, occlusive retinal vascular disease, retinopathy of prematurity (ROP), choroidal rupture, ocular histoplasmosis syndrome (POHS), toxoplasmosis, and Leber's congenital amaurosis. None of the above lists is exhaustive.

Many ophthalmic diseases, such as (age-related) macular degeneration, macular dystrophies such as Stargardt's and Stargardt's-like disease, Best disease (vitelliform macular dystrophy), and adult vitelliform dystrophy or subtypes of retinitis pigmentosa, are associated with a degeneration or deterioration of the retina itself or of the RPE. It has been demonstrated in animal models that photoreceptor rescue and preservation of visual function could be achieved by subretinal transplantation of RPE cells (Coffey et al. Nat. Neurosci. 2002:5, 53-56; Lin et al. Curr. Eye Res. 1996:15, 1069-1077; Little et al. Invest. Ophthalmol. Vis. Sci. 1996:37, 204-211; Sauve et al. Neuroscience 2002:114, 389-401). There is a need to find ways to produce RPE cells, such as from human stem cells, that can be used for the treatment of retinal degenerative diseases and injuries.

Age-related Macular degeneration (AMD) is the most common cause of blindness in elderly population. There are two types of AMD, a dry form that results in RPE atrophy and a wet form that results in abnormal growth of choroidal vasculature that penetrates the RPE. The dysfunctional RPE has been associated with disease pathology and progression as it is unable to support photoreceptor which leads to the degeneration of neural retinal layer and hence vision loss. Currently there are only treatments available for the wet form of AMD which include laser coagulation therapy and anti-VEGF injections. Similarly, to date there are no effective treatments for retinal degenerative diseases like proliferative viteroretinopathy (PVR) and age-related and inherited retinal degenerations that is characterized by the loss of epithelial phenotype in RPE EMT cells eventually leading to blindness.

There continues to be a need for compounds and methods useful in the treatment retinal degenerations. Such treatment would prevent or reduce the rate of retinal degeneration arising from multiple etiologies.

### BRIEF SUMMARY OF THE INVENTION

In one aspect, the invention provides a method of treating a retinal disease comprising administering to a patient in need thereof a pharmaceutically effective amount of a compound, or a pharmaceutically acceptable salt thereof, which inhibits Nox4 or reactive oxygen species formation, or modulates serine protease, a dopamine receptor, NF-kB, mTOR, AMPK, RPE epithelial to mesenchymal transition, RPE dedifferentiation, or one or more Rho GTPases.

In certain embodiments of the method of treating a retinal disease of the invention, the retinal disease is macular or peripheral retinal degeneration, retinal pigment epithelium atrophy, macular dystrophy, Geographic Atrophy, choroidal neovascularization, Stargardt's disease, a Stargardt's-like disease, Best disease, vitelliform macular dystrophy, adult vitelliform dystrophy, retinitis pigmentosa, proliferative vitreoretinopathy, retinal detachment, pathologic myopia, diabetic retinopathy, CMV retinitis, occlusive retinal vascular disease, retinopathy of prematurity (ROP), choroidal rupture, ocular histoplasmosis syndrome (POHS), toxoplasmosis, or Leber's congenital amaurosis.

In other embodiments of the method of treating a retinal disease of the invention, the compound is a Nox4 inhibitor (or reactive oxygen species inhibitor). In still other embodiments of the method of treating a retinal disease of the invention, the compound modulates NF-kB, mTOR, or one or more Rho GTPases. In specific embodiments, the compound modulates one or more Rho GTPases, the Rho GTPase is CDC42 and/or RAC1. In other embodiments, wherein the compound modulates AMPK. In still other embodiments, the compounds regulate RPE epithelial to mesenchymal transition or RPE dedifferentiation.

In certain embodiments of the method of treating a retinal disease of the invention, the compound is Aminocapropic acid, L-701,324, Vas2870, L-745,870 hydrochloride, Me-3,4-dephostatin, N-Methyl-1-deoxynojirimycin, L-750,667 trihydrochloride, (+)-MK-801 hydrogen maleate, Pempidine tartrate, (−)-Naproxen sodium, Raloxifene hydrochloride, SKF 83959 hydrobromide, L-687,384 hydrochloride, 7,7-Dimethyl-(5Z,8Z)-eicosadienoic acid, SP-600125, Ro 41-0960, Ancitabine hydrochloride, Risperidone, Telenzepine dihydrochloride, NO-711 hydrochloride, U-99194A maleate, S(+)-Raclopride L-tartrate, Pirenzepine dihydrochloride, Captopril, Thioperamide maleate, Alprenolol hydrochloride, Ritodrine hydrochloride, Putrescine dihydrochloride, 1-(2-Methoxyphenyl)piperazine hydrochloride, PAPP, U-69593, AG-1478, riluzole, Phentolamine mesylate, DBO-83, Formestane, Carbamazepine, 4-(2-Aminoethyl)benzenesulfonyl fluoride hydrochloride, Terbutaline hemisulfate, UK 14304, GR 113808, Leflunomide, Acetylthiocholine chloride, spermidine, 5-(N-Methyl-N-isobutyl)amiloride, ATPO, Acadenisine or Metformin, or a combination thereof. In particular embodiments of the method of treating a retinal disease of the invention, the compound is Aminocaproic Acid; L-745,870; Riluzole; Acadenisine; Metformin or a pharmaceutically acceptable salt thereof.

In some embodiments of the method of treating a retinal disease of the invention, the compound is administered in the form of a pharmaceutical composition wherein the pharmaceutical composition comprises the compound and one or more pharmaceutically acceptable carriers.

In another aspect, the invention provides, a method of treating retinal degeneration comprising administering to a patient in need thereof a pharmaceutically effective amount of a compound, or a pharmaceutically acceptable salt thereof, which inhibits Nox4, or modulates NF-kB, mTOR, AMPK, RPE epithelial to mesenchymal transition, or RPE dedifferentiation, or one or more Rho GTPases.

In certain embodiments of the method of treating a retinal degeneration of the invention, the compound is a Nox4 inhibitor (or reactive oxygen species inhibitor). In still other embodiments of the method of treating a retinal degeneration of the invention, the compound modulates NF-kB, mTOR, or one or more Rho GTPases. In specific embodiments, the compound modulates one or more Rho GTPases, the Rho GTPase is CDC42 and/or RAC1. In other embodiments, wherein the compound modulates AMPK. In still other embodiments, the compounds regulate RPE epithelial to mesenchymal transition or RPE dedifferentiation.

In certain embodiments of the method of treating a retinal degeneration of the invention, the compound is Aminocapropic acid, L-701,324, Vas2870, L-745,870 hydrochloride, Me-3,4-dephostatin, N-Methyl-1-deoxynojirimycin, L-750,667 trihydrochloride, (+)-MK-801 hydrogen maleate, Pempidine tartrate, (−)-Naproxen sodium, Raloxifene hydrochloride, SKF 83959 hydrobromide, L-687,384 hydrochloride, 7,7-Dimethyl-(5Z,8Z)-eicosadienoic acid, SP-600125, Ro 41-0960, Ancitabine hydrochloride, Risperidone [Please Confirm], Telenzepine dihydrochloride, NO-711 hydrochloride, U-99194A maleate, S(+)-Raclopride L-tartrate, Pirenzepine dihydrochloride, Captopril, Thioperamide maleate, Alprenolol hydrochloride, Ritodrine hydrochloride, Putrescine dihydrochloride, 1-(2-Methoxyphenyl)piperazine hydrochloride, PAPP, U-69593, AG-1478, riluzole, Phentolamine mesylate, DBO-83, Formestane, Carbamazepine, 4-(2-Aminoethyl)benzenesulfonyl fluoride hydrochloride, Terbutaline hemisulfate, UK 14304, GR 113808, Leflunomide, Acetylthiocholine chloride, spermidine, 5-(N-Methyl-N-isobutyl)amiloride, ATPO, Acadenisine or Metformin, or a combination thereof. In particular embodiments of the method of treating a retinal degeneration of the invention, the compound is Aminocaproic Acid; L-745,870; Riluzole; Acadenisine; Metformin or a pharmaceutically acceptable salt thereof.

In some embodiments of the method of treating a retinal degeneration of the invention, the compound is administered in the form of a pharmaceutical composition wherein the pharmaceutical composition comprises the compound and one or more pharmaceutically acceptable carriers.

In still another aspect, the invention provides a method of restoring retinal pigment epithelium cells comprising administering to a patient in need thereof a pharmaceutically effective amount of a compound, or a pharmaceutically acceptable salt thereof, which inhibits Nox4, or reactive oxygen species formation, or modulates serine protease, a dopamine receptor, NF-kB, mTOR, AMPK, RPE epithelial to mesenchymal transition, or RPE dedifferentiation, or one or more Rho GTPases.

In certain embodiments of the method of restoring retinal pigment epithelium cells the invention, the retinal disease is disorder is macular degeneration, retinal pigment epithelium atrophy, macular dystrophy, Stargardt's disease, a Stargardt's-like disease, Best disease, vitelliform macular dystrophy, adult vitelliform dystrophy, retinitis pigmentosa, proliferative vitreoretinopathy, retinal detachment, pathologic myopia, diabetic retinopathy, CMV retinitis, occlusive retinal vascular disease, retinopathy of prematurity (ROP), choroidal rupture, ocular histoplasmosis syndrome (POHS), toxoplasmosis, or Leber's congenital amaurosis.

In other embodiments of the method of restoring retinal pigment epithelium cells, the compound is a Nox4 inhibitor. In still other embodiments of the method of treating a retinal disease of the invention, the compound modulates NF-kB, mTOR, or one or more Rho GTPases. In specific embodiments, the compound modulates one or more Rho GTPases, the Rho GTPase is CDC42 and/or RAC1. In other embodiments, wherein the compound modulates AMPK. In still other embodiments, the compounds regulate RPE epithelial to mesenchymal transition or RPE dedifferentiation.

In certain embodiments of the method of restoring retinal pigment epithelium cells, the compound is Aminocapropic acid, L-701,324, Vas2870, L-745,870 hydrochloride, Me-3,4-dephostatin, N-Methyl-1-deoxynojirimycin, L-750,667 trihydrochloride, (+)-MK-801 hydrogen maleate, Pempidine tartrate, (−)-Naproxen sodium, Raloxifene hydrochloride, SKF 83959 hydrobromide, L-687,384 hydrochloride, 7,7-Dimethyl-(5Z,8Z)-eicosadienoic acid, SP-600125, Ro 41-0960, Ancitabine hydrochloride, Risperidone [Please Confirm], Telenzepine dihydrochloride, NO-711 hydrochloride, U-99194A maleate, S(+)-Raclopride L-tartrate, Pirenzepine dihydrochloride, Captopril, Thioperamide maleate, Alprenolol hydrochloride, Ritodrine hydrochloride, Putrescine dihydrochloride, 1-(2-Methoxyphenyl)piperazine hydrochloride, PAPP, U-69593, AG-1478, riluzole, Phentolamine mesylate, DBO-83, Formestane, Carbamazepine, 4-(2-Aminoethyl)benzenesulfonyl fluoride hydrochloride, Terbutaline hemisulfate, UK 14304, GR 113808, Leflunomide, Acetylthiocholine chloride, spermidine, 5-(N-Methyl-N-isobutyl)amiloride, ATPO, Acadenisine or Metformin, or a combination thereof. In particular embodiments of the method of treating a retinal disease of the invention, the compound is Aminocaproic Acid; L-745,870; Riluzole; Acadenisine; Metformin or a pharmaceutically acceptable salt thereof.

In some embodiments of the restoring retinal pigment epithelium cells, the compound is administered in the form of a pharmaceutical composition wherein the pharmaceutical composition comprises the compound and one or more pharmaceutically acceptable carriers.

In another aspect, the invention provides a method of treating Stargardt's disease or a Stargardt's-like disease comprising administering to a patient in need thereof a pharmaceutically effective amount of a compound or a pharmaceutically acceptable salt thereof, wherein the compound is Aminocaproic Acid, Vas2870, L-745,870, Riluzole, Acadenisine, or Metformin. In some embodiments of the method of treating Stargardt's disease or a Stargardt's-like disease of the Mention, the compound is Metformin or a pharmaceutically acceptable salt thereof.

In some embodiments of the method of treating Stargardt's disease or a Stargardt's-like disease of the invention, the compound is administered in the form of a pharmaceutical composition wherein the pharmaceutical composition comprises the compound and one or more pharmaceutically acceptable carriers.

In particular embodiments of the method of treating Stargardt's disease or a Stargardt's-like disease of the invention, the compound or composition of the invention is administered topically to the eye of the subject, or administered to the subject through intravitreous injection, sub-tenon injection, or sub-retinal injection.

In particular embodiments of the method of treating Stargardt's disease or a Stargardt's-like disease of the invention, the compound or composition of the invention is administered topically to the eye of the subject, or administered to the subject through intravitreous injection, sub-tenon injection, or sub-retinal injection.

In particular embodiments of the method treating a retinal disease of the invention, the compound or composition of the invention is administered topically to the eye of the subject, or administered to the subject through intravitreous injection, sub-tenon injection, or sub-retinal injection.

In particular embodiments of the method of treating retinal degeneration of the invention, the compound or composition of the invention is administered topically to the eye of the subject, or administered to the subject through intravitreous injection, sub-tenon injection, or sub-retinal injection.

In particular embodiments of the method of restoring retinal pigment epithelium cells degeneration of the invention, the compound or composition of the invention is administered topically to the eye of the subject, or administered to the subject through intravitreous injection, sub-tenon injection, or sub-retinal injection.

### BRIEF DESCRIPTION OF THE DRAWINGS

FIGS. 1a-1s depict various testing data which demonstrates complement competent human serum (CC-HS) induces AMD-like cellular endophenotypes in mature iRPE. (a) mature and polarized iRPE cells with F-ACTIN (green) and β-CATENIN (red) co-localized at the cell borders were used throughout the study, n=3. (b-d) iRPE treated with CC-HS for 48 h showed a significant 10-fold increase in APOE positive sub-RPE deposits, as compared to complement incompetent human serum (CI-HS); n=10 different iRPE lines with three technical replicate for each line. (e, f) CC-HS also upregulated the neutral lipid droplets as compared to CI-HS treatment, as observed by staining with Nile red, n=3. (g, h) transmission electron microscopy (TEM) confirmed sub-RPE laminar and lipid deposits to only be present in CC-HS treated iRPE (arrow in h), n=10. (i,j) CC-HS treatment induced stress fibers in iRPE, as seen by F-actin staining (yellow) (arrowheads in J), and a loss of RPE hexagonality, n=3. (k-o) Loss of junctional integrity in CC-HS treated iRPE cells. (k,l) mis-localized expression of tight junction marker, CLDN16 (red) in the cytoplasm co-stained with another junctional marker, Na+/K+ ATPase (green) (arrowhead in L as compared to K), n=3. (m,n) TEM of CC-HS treated iRPE confirmed the disappearance of tight junctions between neighboring RPE cells (arrowheads in n when compared to arrowheads in m), n=3. (o) box-plot shows a significant three-fold decrease in TER measurements for CC-HS treated iRPE monolayers, with respect to CI-HS treated cells. (p) Represents the loss of iRPE functionality in CC-HS treated cells, assessed in terms of phagocytic ability. The CC-HS treatment resulted in a significant six-fold drop in uptake of photoreceptor outer-segments. (q-s) CC-HS treatment induced loss of iRPE response to physiological stimuli such as ATP and 1 mM K+, when compared to CI-HS samples. The representative traces of altered responses in stressed iRPE cells with respect to healthy cells are shown in (q and r).

FIGS. 2a-2g depict various testing data which demonstrates CC-HS induced AMD like cellular endophenotypes likely works through C5a and C3a signaling. (a) Western blot confirms the complement receptor 3a (C3aR) and complement receptor 5a (C5aR) to be present in the membrane fraction of iRPE cell lysates only, with no expression in cytoplasmic fraction, liver and A549 cell line lysates serves as positive controls. Na+/K+ ATPase, a known membrane protein marker acts a loading control. (b,c) The co-localization of C5aR1, C3aR1 (red) with ezrin (green, apical processes marker, top panel) and not with Collagen V (green, basal marker, bottom panel) verifies the presence of receptors on the apical side of iRPE cells. (d-g) Total ERK1/2, AKT, and phosphoylated p-ERK1/2, p-AKT protein levels in iRPE cell lysates were checked by WB across three different iRPE cell lines treated in CI-HS, or CC-HS, quantifications are shown in the bar graph.

FIGS. 3a-3k depict various testing data which demonstrates activation of NF-kB pathway acts downstream of C5aR1 and C3aR1 signaling by inducing AMD like cellular endophenotypes. (a-b) Treatment of iRPE with CC-HS, compared to CI-HS, induced a translocation of the p65 subunit (stained in red, b) from the cytosol to the nucleus indicating the activation of the NF-kB pathway. (c) qRT-PCR further confirmed an increased expression of target and pathway genes of the NF-kB pathway, in CC-HS treated iRPE. (d, e) confirms the increased apical and basal secretion of IL-8 and IL-18 in CC-HS treated cells suggesting the activation of the pathway. (f-h) Nuclear translocation of p65 (red) is not seen in iRPE treated with C3, C5 or CFD depleted human serum, suggesting that complement proteins C3 and C5 have integral roles in the activation of NF-kB in CC-HS treated iRPE cells. (i-k) The iRPE from the patient with a mutation in NEMO—a negative regulator of NF-kB pathway, was examined to confirm the role of the NF-kB pathway. (i) shows increased basal levels of p65 in the nucleus, (j-k) The patient line showed more APOE positive sub-iRPE deposits, and quantification of the data is shown in figure k.

FIGS. 4a-4j depict various testing data which demonstrates that anaphylatoxin complement downregulates autophagy in iRPE cells. (a-f) Autophagy proteins, LC3 (red, a,b) and ATG 5 (red, d,e), are downregulated in CC-HS treated iRPE, compared to CI-HS treated iRPE. (c,f) Quantification of Western blots confirms 2× reduced expression for LC3-II (i) and ATG5 (1) in CC-HS treated samples, as compared to CI-HS treated samples. (g) qRT-PCR reveals 3-6 fold reduced expression of multiple autophagy pathway genes in CC-HS treated iRPE, compared to CI-HS treated cells. (h,i) The accumulation of autophagolysosomes is induced with CC-HS treatment on iRPE, but not with CI-HS treatment. (j) Western blot confirms that induced reduction of LC3-II expression with CC-HS treatment is not present in iRPE treated with C5 and C3 depleted human serum or with C5aR+C3aR blocked cells treated with CC-HS.

FIGS. 5a-5f depict various testing data which demonstrates that the proteotoxic high throughput screen identifies drugs that rescue iRPE health. (a) Calcium response curves in CI-HS and (b) CC-HS treated iRPE cells. (c) A23187 is a proteotoxic drug that kills iRPE over a 48 h period. 10 μM A23187 concentration kills approximately 40% cells in 48 h. Dot plot shows results from two different sets of 384-well plates. Plates 3-6 iRPE were treated with 10 um A23187 and 46 uM of 1280 Library of Pharmaceutically Active Drugs (LOPAC) drugs, whereas in plates 7-10, iRPE were treated with 10 uM A23187 and 9.2 uM of LOPAC. Percent cell survival was scored using CellTitrGlow (ATP release) assay and plotted on the Y-axis. Note, at a concentration of 46 um, most drugs are cytotoxic, whereas at a 9.2 uM concentration range, approximately 20 drugs improved iRPE cell survival to varying degrees. (d-f) The seven-point dose curve of four drugs (L745,870—d; riluzole—e; aminocaproic acid—f) shows reproducible cell survival between two iRPE samples, over three different A23187 concentrations (2.5 uM, red; 5 uM, blue; and 10 uM, green).

FIGS. 6a-6k depict various testing data which demonstrates that anti-proteotoxic drugs ameliorate the effects of CC-HS on iRPE and rescue RPE cell health and functions. (a-e) Co-treatment of iRPE with drugs (Riluzole, L745, 470, and aminocaproic acid) and CC-HS does not lead to nuclear translocation of the p65 subunit of Nf-kB (red) (a-e), or reduced expression of autophagy protein, ATG5 (red) (f-j).

FIGS. 7a-7h depict various testing data which demonstrates that anti-proteotoxic drugs suppress NF-kB activation and upregulate autophagy in CC-HS treated iRPE cells. (a, b) Co-treatment of CC-HS treated iRPE cells with L-745,870 (L-745) or aminocaproic acid (ACA) reduced the amount of Nile red positive lipid droplets (a) and the expression of Fibulin3 (b), compared to CC-HS and vehicle treated cells. (c-f) Co-treatment of CC-HS treated iRPE cells with L-745 and ACA reduced area (c, e) and improved hexagonality (d, f) of CC-HS treated iRPE cells (c, d), and RPE cells at the borders of laser lesion in rat eyes (e, f). (g, h) Co-treatment of CC-HS treated iRPE cells with L-745 and ACA increased monolayer TER (g) and phagocytic ability (h).

FIGS. 8a-8b depicts Sschematic of changes in iRPE phenotype following CI-HS or CC-HS treatment.

FIGS. 9a-9m depict various testing data which demonstrates complement competent human serum (CC-HS) treatment leads to basal RPE deposits. (a) Progressive increase in transepithelial resistance (TER) indicates maturity of iRPE monolayers. (b) CC-HS treated transwell membrane co-stained for APOE (red) and C5ab (green). (c, d) Immunostaining reveals increased FIBULIN3 (green) expression in CC-HS treated iRPE, compared to CI-HS treated iRPE. (e, f) Oil red O staining (red) reveals higher number of intracellular lipid droplets in CC-HS treated, compared to CI-HS treated iRPE. (g, h) Scanning electron micrographs (SEM) of the basal surface of the iRPE reveal increased laminar deposits following CC-HS treatment (red arrow heads). (i-I) Immunostaining shows altered Vimentin (red) expression, without any cytoskeleton structure in CC-HS treated iRPE (j) and at the borders of GA lesion in an AMD eye (1). CI-HS treated cells (i) and non-lesions RPE (k) areas show normal membranous and cytoplasmic organized with cytoskeleton expression of Vimentin. F-actin (green) markers actin cytoskeleton. (m) Quantification of electrophysiology data shows that CC-HS treatment leads to 2.5× lower TER under resting state, and dampened TER changes under 1 mMK and ATP stimuli.

FIGS. 10a-10m depict various testing data which demonstrates Anaphylatoxin complement proteins mediate AMD-like cellular endophenotypes in iRPE. (a) mRNA expression levels for C3aR1 and C5aR1 in primary and iRPE cells. Note, ˜30× higher expression of C5aR1 in iRPE cells. Expression of both receptors increases with CC-HS treatment. (b) Immunostaining of iRPE cells predominant apical expression of C3aR1 (left panels, red) and C5aR1 (right panels, red) in iRPE cells as confirmed by co-localization with EZRIN (green, apical marker), and minimal with Collagen IV (green, basal marker). (c-f) In cadaver human eyes, both C3aR1 and C5aR1 are expressed similarly on apical and basal sides of RPE cells, as confirmed by co-localization with EZRIN (green, apical marker) and with Collagen IV (green, basal marker). There is also a prominent intracellular expression of these receptors in RPE cells. (g-k) APOE staining of iRPE membranes treated with human sera depleted of proteins CFD (upstream of C3 and C5), C3, C5, and co-treated CC-HS plus receptor blockers for C3aR1 and C5aR1 show reduced APOE expression under all conditions, compared to CC-HS treatment. (1, m) Electrophysiological responses of iRPE treated with human sera depleted in C5 and C3 proteins display normal electrical properties of RPE cells.

FIGS. 11a-11e depict various testing data which demonstrates that RNAseq identifies upregulation of NF-kB target genes and downregulation of autophagy genes in CC-HS treated iRPE as compared to CI-HS treated cells. (a) Heatmap of RNAseq data from three different donor derived iRPE samples reveals clustering of samples by CI-HS and CC-HS treatments. (b) Top ten pathways statistically different gene expression pattern in CI-HS vs CC-HS treated iRPE. (c) Heatmap of RNAseq data identifies an upregulation of NF-KB target genes in CC-HS treated iRPE, compared to CI-HS treated iRPE. (d) Immunostaining shows increased expression of NF-KB target genes RELB (red) and TRAF3 (red) in CC-HS treated iRPE compared to CI-HS treated cells. (e) Two-fold upregulation in apical and basal secretions of IL-18 an NF-KB downstream cytokine in CC-HS treated iRPE.

FIGS. 12a-12f depict various testing data which demonstrates that anaphylatoxin complements downregulate autophagy in iRPE. (a, b) Western blots show reduced levels of autophagy proteins, ATG5 (a), ATG7 (a), and LC3-II (b), across three different donor derived CC-HS treated iRPE samples. β-ACTIN was used for normalization. (c) 3-6-fold decreased expression of autophagy pathway genes in CC-HS treated iRPE, compared to CI-HS treated iRPE. (d) TEM shows accumulation of autophagolysosomes (red arrow heads) in CC-HS treatment on iRPE (e) Western blots show LC3-II expression levels reduced only in iRPE samples treated with CC-HS on the apical side, or both sides, and not when treated only on the basal side. β-ACTIN was used for normalization. (f) Western blots showed similar LC3-II expression levels across CI-HS treated iRPE, iRPE treated with C5 or C3 depleted human serum, and iRPE co-treated with CC-HS and C5aR1+C3aR1 receptor blockers. β-ACTIN was used for normalization. (g-u) time dependent activation of NF-kB pathway (g-k), downregulation of autophagy (l-q), and APOE deposit formation (r-u) in CC-HS treated iPSC-RPE cells.

FIGS. 13a-13g depict various testing data which demonstrates that proteotoxic high throughput screen with iRPE cells. (a) At 96 h, all three concentrations of A23187 (2.5 μM, 10 μM, 25 μM) are cytotoxic on iRPE cells. (b) Mean relative light intensity across all the 10 plates shows similar results across all plates treated with A23187, suggesting screen reproducibility across different plates. (c) Percent cell killing by A23187 is similar across all the plates with slight reduction in plates treated with 9.2 μM drug, suggesting cell survival in those plates. (d-f) heat maps of secondary screen using three different A23187 concentrations (2.5 μM—e, 10 μM—f, 25 μM—g) and seven different concentrations of drugs ranging from (10 μM to 10 μM). Responses of four select drugs are highlighted. (g) 45 drugs were selected from the primary screen for a hit-validation in a follow-up screen. Four drugs (L745,870, AG-1478, riluzole, and aminocaproic acid) were selected in the follow up screen based on a linear response the seven-point dose curve and reproducibility between two different iRPE samples. (h) PCA plot shows separate clustering for iRPE treated with drugs and CC-HS, only CC-HS, and only CI-HS. (i, j) Heatmaps of RNA seq data for iRPE derived from three donors and three treatment groups (CI-HS vs CC-HS, CC-HS+ vehicle vs CC-HS+L, 745,870 or ACA) shows a reversal of the upregulation of NF-KB pathway genes and a reversal in downregulation of autophagy genes in samples co-treated with drugs and CC-HS as compared to samples treated with CC-HS.

FIGS. 14a-14i depict various testing data which demonstrates that patient-specific iPSC-RPE retained a disease-causing mutation. (a) Sanger sequence analysis confirms the presence of the S163R mutation in iPSCs derived from patients with L-ORD. The sequences are shown on top and the base affected by the mutation is indicated on the sequence chromatogram by the black arrow. The heterozygous point mutation (AGC->AGC, AGG) appears as a peak within a peak. Primers for DNA sanger sequencing are described in Methods. (b) boxplot diagrams of deltaCt values of the indicated RPE signature genes. Each box represents the distribution of the deltaCt measured from n=3 iPSC-RPE from at least 2 different unaffected siblings or L-ORD patient donors. Bottoms and tops of the boxes define the 10^{th} and 90^{th} percentile. The band inside the box defines the median. (c) Transmission electron microscopy images of iPSC-RPE monolayers fed photoreceptor outer segments for 7 consecutive days. TEM of iPSC-RPE derived from an unaffected sibling (above) and patient (below) showing normal RPE morphology and highly polarized structure including abundant apical processes (yellow arrow), melanosomes (magenta arrow), and basally located nuclei (white arrow). Scale bar: 2 μm. (d) SEM images of iPSC-RPE derived from unaffected siblings and L-ORD patients showing preserved hexagonal morphology and abundant apical processes. (e) Box plot of cell area of iPSC-RPE derived from unaffected siblings and L-ORD patients. iPSC-RPE monolayers were immunostained with a membrane marker (ADIPOR1) to outline their hexagonal shape for multiparametric analysis of cell morphology. L-ORD patient iPSC-RPE tended to be larger in size on average (107.7+/−68.5 μm²) and more variable compared to unaffected siblings (79.8+/−57.5 μm²) (p=0.000026). Similar spatial irregularities have been reported in the eyes of human AMD donors. (f) Establishment of functional tight junctions between iPSC-RPE cells was measured by transepithelial resistance measurements using an EVOM epithelial voltohmmeter (World Precisions Instruments). The disease associated missense mutation does not alter the transepithelial resistance of the RPE monolayer. (g) Scatter plot of genes enriched in RPE cells that undergo dedifferentiation (epithelial mesenchymal transition) reveal that under normal conditions L-ORD patient cells do not show an abnormal phenotype indicative of diseased or stressed RPE. The expression of dedifferentiation (EMT)-related genes in unfed (shown in gray) patient iPSC-RPEs resemble the expression patterns of unfed unaffected siblings. (h) iPSC-RPE derived from unaffected siblings and L-ORD patients subjected to normal culture conditions show similar levels of APOE basal deposits. Scale bar: 50 μm. (i) The release of VEGF by iPSC-RPE into the supernatant under normoxic conditions was measured by ELISA. The highly polarized structure of RPE is responsible for vectorial transport and secretion of proteins including VEGF. Naturally, iPSC-RPE derived from unaffected siblings (shown in gray) secreted VEGF in a polarized manner, predominantly basal. L-ORD Patient derived iPSC-RPE exhibit a loss of polarity with approximately a ˜53.3% reduction in basal VEGF secretion (P=0.046).

FIGS. 15a-15h depict various testing data which demonstrates expression and localization of CTRP5 in L-ORD patient-derived RPE. (a) In L-ORD the S163R mutation occurs in a bicistronic transcript that codes for CTRP5 (a secretory protein) and membrane frizzled related protein (MFRP). The mutation does not alter the mRNA expression of either transcript. (b) Representative western blot of cell lysate from iPSC-RPE of unaffected siblings and L-ORD patients. Since CTRP5 is a secreted protein, the strong 25 kDa band (CTRP5) in the unaffected siblings may indicate CTRP5 is retained to a greater degree in the whole cell extract. (c) Quantification of western blot (cell lysate) normalized to β-actin (p<0.05). (d) In iPSC-RPE from unaffected siblings and L-ORD patients CTRP5 was selectively secreted to the apical side as measured by ELISA following 48 hours. No measureable difference was observed between the amounts secreted by unaffected siblings and patients. Negligible amounts of CTRP5 were detected in the basal media (data not shown). (e) Airyscan confocal microscopy images of immunofluorescent stainings of iPSC-RPE from unaffected siblings and L-ORD patients. The membrane receptor ADIPOR1 (shown in green) co-localizes with CTRP5 (shown in red), HOESCHT (nuclear stain shown in blue). (f) TEM image of native immunolabeled ADIPOR1 (6 nm immunogold) and CTRP5 (12 nm immunogold) provide evidence of receptor-ligand interaction (indicated by black arrow). (g) 3-D model of protein-protein interaction between ADIPOR1 (shown in blue) and CTRP5 (shown in green) using published crystallographic structures. h) The Serine (polar) to Argenine (+) mutation alters the charge of the residue making it positive. This positive charge is predicted to repel a neighboring argenine residue and results in a conformational change that reduces the binding affinity of the mutant CTRP5 to ADIPOR1.

FIGS. 16a-16f depict various testing data which demonstrates reduced antagonism of CTRP5 on ADIPOR1 results in altered AMPK signaling in L-ORD. (a) Phospho-AMPK levels determined by ELISA indicate approximately a 20.6% increase in baseline activity in L-ORD patient iPSC-RPE (N=15; (120.6%±0.075) cultured in 5% serum containing media compared to unaffected siblings (N=21; 100%±0.04). (b) Influence of recombinant globular CTRP5 on phospho-AMPK levels in the presence and absence of serum containing adiponectin. Data are normalized to the untreated condition (0 ug/mL gCTRP5). In unaffected siblings, the addition of 0.2 μg/mL of recombinant globular CTRP5 in the absence of the natural ligand, adiponectin (under 0% serum conditions) reveals a 20% decrease in pAMPK levels (N=9; 0.81±0.04). This significant decrease is masked by the presence of 5% serum under baseline conditions (N=6; 0.99±0.01). In L-ORD patient iPSC-RPE, the addition of 0.2 μg/mL recombinant globular CTRP5 has no measurable effect on the p-AMPK levels (N=6; 1.12±0.09) even in the absence of serum (N=6; 0.98±01). (c) Dose-response effects of recombinant full length CTRP5 on the p-AMPK levels of iPSC-RPE derived. In unaffected siblings (5h 0% serum), the phosphorylation levels of AMPK are reduced after treatment (30 min) with increasing concentrations of recombinant full length CTRP5. 25 ug/mL CTRP5 results in a ˜50% reduction in p-AMPK levels (N=6, 47.89%±0.13). Patient RPE subjected to similar concentrations of full length CTRP5 elicited no measurable change in p-AMPK levels. (d) Conditions that elevate the AMP:ATP ratio in the absence of serum result in altered p-AMPK levels in patient derived iPSC-RPE compared to unaffected siblings. All data are normalized to the 0% serum containing condition. 30 min treatment with 2 mM AICAR, an AMP analog, or 500 nM BAM15, a mitochondrial uncoupler that reduces ATP production, results in further elevation in AMPK levels in unaffected siblings. In contrast the p-AMPK levels of patient RPE are insensitive to changes in AMP or ATP levels. However two-week treatment with 3 mM metformin restores the sensitivity of the L-ORD patients to changes in the AMP:ATP ratio. (e) Elevated AMPK in L-ORD patient derived iPSC-RPE results in significantly upregulated mRNA expression of PEDF-R (˜8-fold). (f) Immunohistochemistry confirmed elevated PEDF-R protein expression localized to the apical membrane in L-ORD patient iPSC-RPE.

FIGS. 17a-17f depict various testing data which demonstrates altered lipid metabolism in L-ORD patients contributes to reduced neuroprotective signaling. (a) Presumptive model depicting the phagocytic uptake of lipid-rich outer segments and their digestion by phospholipase into free fatty acids that the RPE utilizes for ketogenesis and the synthesis of neuroprotective lipid mediators such as NPD1. In human cancer cell lines, elevated p-AMPK levels have been shown to suppress phospholipase D activity and is the proposed mechanism through which increased lipid uptake in L-ORD patients results in decreased utilization and synthesis of DHA-derived Neuroprotectin D1 and an accumulation of undigested lipids. (b) The uptake of ph-Rhodo labeled outersegments were quantified by FACS to compare the phagocytic rate of iPSC-RPE derived from unaffected siblings and L-ORD patients. The phagocytic uptake of L-ORD patient iPSC-RPE (N=14; 11.81±3.55) was 33% higher than unaffected siblings (N=15; 7.86±3.94). This phenomenon of increased lipid uptake has been reported in RPE as a protective response to oxidative stress. (c, d) Despite a significant increase in overall PEDF-R expression, L-ORD patient phospholipase A2 activity was measured by ELISA to be 40% lower than unaffected siblings. (e) Phospholipase A2 activity is shown to be significantly reduced (26%) in normal iPSC-RPE (n=6) subjected to elevated levels of pAMPK (n=6, induced by serum starvation) (p<0.05). (f) The polarized secretion of PEDF was determined by ELISA. L-ORD patients (N=12) exhibited reduced apical (patient: 939.6 ng/mL/sibling: 1277.22 ng/mL) and increased basal (patient: 92.16 ng/mL/sibling: 75.96 ng/mL) secretion of PEDF, resulting in a significantly reduced PEDF ratio (Ap/Ba) (10.13±1.63) compared to unaffected siblings (N=12, 19.82±3.67) (p=0.0014). Data are mean±SE and represent the average of 3 independent experiments. * indicates is p<0.05. f) Apical secreted DHA-derived neuroprotection D1 was measured by tandem mass spectrometry lipidomic analysis. Unaffected siblings (Z8: n=12, 9i: n=12) collected and pooled over 6 days secreted approximately ˜10 times more NPD1 than L-ORD patients (K8: n=12, E1: n=12) (p=0.0089).

FIGS. 18a-18h depict various testing data which demonstrates L-ORD patient RPE have increased susceptibility to epithelial-mesenchymal transition. (a, b) All images were obtained using a 63× objective. Scale bar=20 um. b) Images obtained under conditions described in were subjected to shapemetric analysis to construct box plots of the distribution of cell area (Low whisker: 5% of data, Low hinge: 25% of data, Midline: Median, High hinge: 75% of data, High whisker: 95% of data). L-ORD Patient iPSC-RPE (N=6 images, 135.37±1.76 um) possess increased cell size and variability compared to unaffected siblings (N=5, 95.77±1.68 um) (p<2E-16). In unaffected siblings, metformin treatment initiated during photoreceptor feeding had minimal effect on cell area (N=7, 93.14±1.56 um) compared with untreated unaffected siblings (p=0.52). However, 3 mM metformin treatment resulted in a significant decrease in patient cell area (N=7, 117.92±0.96 um) compared to untreated patients (p<2E-16). Dunnett's multiple comparison test was performed to compare either to untreated unaffected siblings or L-ORD patients. (c) Immunofluorescent microscopy images of APOE stained cryosections of iPSC-RPE monolayers following 7-days POS feeding. L-ORD patient iPSC-RPE exhibited altered relative proportions of apical and basal APOE deposition (white arrow). L-ORD patients treated with metformin during POS feeding resulted in a redistribution of the relative proportions of apical and basal APOE deposition (yellow arrow) resembling unaffected siblings. (d) Image quantification of the integrated density of APOE signals of images similar to those shown in c). Integrated density of APOE signal is significantly higher in untreated L-ORD patients (N=5; Apical: 185.69±5.42; Basal: 46.38±2.51) compared to unaffected siblings (N=4; Apical 30.89±12.05; Basal: 8.45±3.09) (Apical: p=7.76E-6; Basal: p=2.71E-5). No significant difference between metformin treated L-ORD patients (N=4; Apical 79.30±37.51; Basal: 13.58±4.58) compared to metformin treated unaffected siblings (N=8; Apical 119.98±20.36; Basal: 23.55±6.17) (Apical: p=0.32; Basal: p=0.32). All images taken at 20×. Scale bar=50 μm. (f) ELISA measurements of VEGF secretion under hypoxic conditions (6h) mimicking from reduced choroidal blood flow has been implicated in the pathophysiology of age-related macular degeneration and serves as a metabolic stressor to determine the susceptibility of L-ORD iPSC-RPE to hypoxia-driven EMT. Similar to normoxic conditions shown in FIG. 1i) L-ORD patient iPSC-RPE (N=10; Ap: 1.89±0.30; Ba: 1.8±0.24) secrete VEGF in a non-polarized manner compared to unaffected siblings (N=9; Ap: 0.78±0.16; Ba: 1.59±0.36) (Ap: p=0.005; Ba: p=0.63). Prior treatment (2 weeks) with metformin protects L-ORD patient RPE (N=6; Ap: 0.59±0.09; Ba: 1.8±0.24) against hypoxia-driven EMT and restores apical/basal VEGF polarity similar to untreated or metformin treated unaffected siblings (N=9; Ap: 0.98±0.16; Ba: 1.64±0.33) (Ap: p=0.09; Ba: p=0.64). (g) The effect of POS feeding on the expression of dedifferentiation (EMT)-related genes in L-ORD patient iPSC-RPE compared to unaffected siblings. 7-days POS feeding (shown in white) causes an increased in the expression of EMT-related genes in L-ORD patients compared to unaffected siblings. Metformin treatment (shown in red) during the 7-days POS feeding suppresses the expression of EMT related genes. Dashed line indicates 4-fold difference. Housekeeping genes: ACTB and GAPDH. (h) Table of results from retrospective clinical study reveals metformin delays age of onset of nonexudative age-related macular degeneration (362.51/H35.31). In patients ages 50-59, metformin delays the age of onset from 56 years of age (n=157, no metformin) to 58.5 years of age (n=16, with metformin) (p=0.001).

FIG. 19 depicts data which demonstrates that the gene expression profile of L-ORD patients suggest a compensatory attempt to limit activation of pAMPK at baseline.

FIG. 20 depicts data which demonstrates that Metformin rescues mispolarized secretion of VEGF in L-ORD Patients RPE under normaxias.

FIG. 21 depicts data which demonstrates that Metformin treatment increased beta-hydroxybutyrate apical secretion by the RPE.

FIGS. 22a-22b depict a model of mechanical retinal injury which mimics the features of RPE-EMT and RPE-dedifferentiation in vivo

FIGS. 23a-23b depicts data which shows that Nox4 is present in the intact RPE, and highly expressed in the injured RPE

FIG. 24 depicts data that demonstrates that NOX4 colocalizes with cytoskeletal proteins known as a EMT markers.

FIG. 25 depicts data that demonstrates that pharmacological inhibition of NOX4 using VAS2870 Down-regulates SMA an EMT marker.

FIGS. 26A-26C depict data showing the knockdown of NOX4 using shRNA.

FIG. 27 depicts data showing that the down-regulation of NOX4 using shRNA decreased cell migration in injured RPE.

FIGS. 28A-28C depict data showing that the down-regulation of NOX4 using shRNA downregulates ZEB1 an EMT marker.

FIG. 29A-29C depict data showing that NOX4 shRNA lentiviral particles successfully downregulates Nestin in scratched RPE.

FIGS. 30A-30B depict data showing that NOX4 effectively downregulates the expression of EMT markers.

FIGS. 31A-31C depict data demonstating ABCA4 localization in RPE cells. A: Western blot analysis of ABCA4 confirms its membrane localization. Membrane (M) and cytoplasmic fractions (C) from human primary (hp) RPE, control iPSC-RPE and fibroblast (negative control). Na/K ATPase is an apical membrane protein in RPE cells. B: ABCA4 and Na/K ATPase co-localization on RPE membrane. C: Orthogonal projections of RPE cells confirm apical co-localization of ABCA4 and Na/K ATPase.

FIGS. 32A-320 depict data demonstrating the characeterization of Stargardt iRPE. A-B: Absence of ABCA4 expression in iRPE (derived from ABCA4−/−, C1 and C2) seen by qRT-PCR (A) and Western blot (B). iPSC—negative control (A, B); monkey retina—positive control (B), Control1—isogenic control for ABCA4−/− iRPE. C: Sanger sequence confirms the presence of the mutation in patient iRPE (C>T in exon 44 at 6088 bp position). D-E: Expression of ABCA4 in patient iRPE by dd-PCR (D) and Western blot analysis(E). F-I: TEM images of control and Stargardt iRPE monolayers show polarized RPE with apical processes, apically located melanosomes, tight junctions, and basally located nuclei. Healthy RPE includes isogenic Control1 for ABCA4−/− clones and Control2 (un-affected unaffected sibling) for the patient iRPE. O Immunostaining of mature RPE markers show similar expression of −/− iRPE and control cells. **p<0.01; ***p<0.001.

FIGS. 33A-33N. depict data demonstating Stargardt pathophysiology replicated in Stargardt iRPE. A-G: Wild type POS fed Stargardt iRPE exhibit increased (2-3-fold) lipid deposits. Comparative analysis of intra/sub-RPE bodipy-positive deposits in un-fed (A-C) and POS-Fed (D-F). Stargardt iRPE exhibited increased (2-3-fold) ceramide accumulation while exposed with POS (J-L). G: Quantitative analysis of lipid deposits (M) and Creamide accumulation (N) in Stargardt-iRPE as compared to control iRPE. Control data point presented here is an average of iRPE from a isogenic Control1 for ABCA4−/−⁺ clones and Control2 for the patient. p<0.05; **p<0.01; ***p<0.001; ****p<0.0001).

FIGS. 34A-34 N depict data showing the effect of ABCA1 KO in ABCA4 lipid handling under complement stress; intra/sub-cellular lipid accumulation in ABCA1KD Stargardt iRPE; and ABCA1 activation rescued lipid accumulation defect in Stargardt RPE. Images of bodipy lipid-positive deposits in CI-HS (A-C) and CC-HS (D-F) treated iRPE cells. G: Quantitative analysis of intra/sub-RPE lipid-positive deposits. As compared to healthy iRPE, Stargardt iRPE shows an 2 increase in bodipy staining (p.ns). Intra/sub-RPE bodipy-positive deposits images of CC-HS (H-J) and CC-HS+GW 3965 (K-M) treated iRPE cells Note reduced lipid deposit in panel K-L when treated with 10 μM GW 3965 (ABCA1 activator). N: Quantitative analysis of intra/sub-RPE bodipy-positive of deposits confirms significant decrease in stargardt iRPE. Control data point presented here is an average of iRPE from a isogenic Control1 for ABCA4−/− clones and Control2 (un-affected unaffected sibling) for the patient. p<0.05; **p<0.01; ***p<0.001; ****p<0.0001).

FIGS. 35A-35B depict data demonstrating POS digestion defect in Stargardt iRPE cells. A: Defects in the clearance of POS and lipofuscin-like accumulation in Stargardt-iRPE. Flow cytometry-based phagocytosis assay reveals similar POS uptake in Stargardt-iRPE compared to healthy-iRPE at 4 h. B: Reduced digestion rate in Stargardt-iRPE. Cells were fed with pHrdho-labeled POS for 4 hrs and were washed with medium after 4h of POS treatment and collected at 4h and 24h for flow cytometry analysis. Healthy RPE includes isogenic Control1 for ABCA4−/− clones and Control2 for the patient iRPE. ***p<0.001).

FIGS. 36A-36C depict data demonstrating that metformin treatment ameliorates disease phenotypes. A: Quantitative analysis of ceramide expression in Stargardt iRPE cells showed a dramatic reduction in its accumulation in POS-Fed Stargardt iRPE treated with metformin. Control data point presented here is an average of iRPE from a isogenic Control1 for ABCA4−/− clones and Control2 for the patient B: lipid distribution in flat-mount images of RPE/Choroid stained with bodipy for Abca4−/− mice treated with metformin. C: The quantification of lipid stain confirms reduced deposits in metformin treated ABCA4 KO mice. (***p<0.001, ****p<0.0001).

### DETAILED DESCRIPTION OF THE INVENTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

All numerical values within the detailed description and the claims herein are modified by “about” or “approximately” the indicated value, and take into account experimental error and variations that would be expected by a person having ordinary skill in the art.

The following terms are used to describe the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. In particular regard to “consisting essentially of,” “consisting essentially of” shall be open-ended to additional components which do not materially effect the compounds or treatments of the invention and shall exclude any additional components which would degrade or otherwise render the compounds of the invention inoperable, which would diminish the effectiveness of the compounds of the invention in the treatments described herein, or which would induce detrimental side effects contrary to the goals of the treatments described herein.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent, such as a chemotherapy or biological therapy that targets epidermal growth factor receptors (e.g., epidermal growth factor receptor inhibitors, such as at least one of gefitinib, erlotinib, neratinib, lapatinib, cetuximab, vandetanib, necitumamab, osimertinib, or a combination thereof). In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented or understood within the context of the compound shown and well-known rules for valence interactions.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

Therapeutic Compounds

The invention provides compounds capable of modulating expression of genes, or proteins or tissues miRNAs or mRNAs or long-non coding RNA which improve morphology, and condition, viability, functionality of retinal pigment epithelium.

In certain embodiments, the compounds of the invention are compounds which inhibit of NADPH-Oxidase 4 (Nox4) function and/or expression or which inhibit formation of radical oxygen species. NADPH oxidases of the Nox family are a group of enzymes whose sole known function is the production of ROS by catalysing electron transfer from NADPH to molecular 02. Four rodent genes of the catalytic subunit Nox (Nox1-4) have been identified, each with tissue-specific expression and different functions in intracellular signalling (Lambeth, 2004; Brown and Griendling, 2009; Zhang et al., 2010).

In certain embodiments, the compounds of the invention are compounds which modulate the expression of serine protease, a dopamine receptor, NF-kB, mTOR, Rho GTPases, CDC42, and/or RAC1, or a combination thereof.

In certain other embodiments, the compounds of the invention are compounds which regulate AMPK.

In still other embodiments, the compounds of the invention modulate RPE epithelial to mesenchymal transition or RPE dedifferentiation.

NF-κB (nuclear factor kappa-light-chain-enhancer of activated B cells) is a protein complex that controls transcription of DNA, cytokine production and cell survival. NF-κB is found in almost all animal cell types and is involved in cellular responses to stimuli such as stress, cytokines, free radicals, heavy metals, ultraviolet irradiation, oxidized LDL, and bacterial or viral antigens. NF-KB plays a key role in regulating the immune response to infection. Incorrect regulation of NF-KB has been linked to cancer, inflammatory and autoimmune diseases, septic shock, viral infection, and improper immune development. NF-κB has also been implicated in processes of synaptic plasticity and memory.

mTOR is a member of the phosphatidylinositol 3-kinase-related kinase family of protein kinases. mTOR links with other proteins and serves as a core component of two distinct protein complexes, mTOR complex 1 and mTOR complex 2, which regulate different cellular processes.

Rho GTPases are molecular switches that control a wide variety of signal transduction pathways in all eukaryotic cells. Rho GTPases are central to dynamic actin cytoskeletal assembly and rearrangement that are the basis of cell-cell adhesion and migration. Human Cdc42 is a small GTPase of the Rho family, which regulates signaling pathways that control diverse cellular functions including cell morphology, cell migration, endocytosis and cell cycle progression. Activated Cdc42 activates by conformational changes p21-activated kinases PAK1 and PAK2, which in turn initiate actin reorganization and regulate cell adhesion, migration, and invasion. Rac1, also known as Ras-related C3 botulinum toxin substrate 1, is a small (˜21 kDa) signaling G protein and is a member of the Rac subfamily of the family Rho family of GTPases. Rac1 is a pleiotropic regulator of many cellular processes, including the cell cycle, cell-cell adhesion, motility (through the actin network), and of epithelial differentiation (proposed to be necessary for maintaining epidermal stem cells).

Serine proteases are a class of enzymes which includes elastase, proteinase 3, chymotrypsin, cathepsin G, trypsin, thrombin, prolyl oligopeptidase and others. A breakdown in the balance of protease/antiprotease activity has been implicated in the pathogenesis of numerous disease states. Serine protease inhibitors encompass a large family of compounds which are capable of regulating, particularly downregulating or inhibiting, serine protease.

Dopamine receptors are a class of G protein-coupled receptors that are prominent in the vertebrate central nervous system (CNS). Dopamine receptors activate different effectors through not only G-protein coupling, but also signaling through different protein (dopamine receptor-interacting proteins) interactions. here are at least five subtypes of dopamine receptors, D1, D2, D3, D4, and D5. Dopamine receptor antagonists encompass a large family of compounds which are capable of modulating, particularly downregulating expression of dopamine receptors.

5′ AMP-activated protein kinase or AMPK or 5′ adenosine monophosphate-activated protein kinase is an enzyme (EC 2.7.11.31) that plays a role in cellular energy homeostasis, largely to activate glucose and fatty acid uptake and oxidation when cellular energy is low. It belongs to a highly conserved eukaryotic protein family and its orthologues are SNF1 and SnRK1 in yeast and plants, respectively. It consists of three proteins (subunits) that together make a functional enzyme, conserved from yeast to humans. Due to the presence of isoforms of its components, there are 12 versions of AMPK in mammals, each of which can have different tissue localizations, and different functions under different conditions.

In certain embodiments, the compound of the invention is a NOX4 inhibitor compound which inhibits formation of a radical oxygen species. In certain other embodiments, the compounds of the invention inhibit or downregulate NF-kB. In other embodiments, the compounds of the invention inhibit or downregulate serine protease. In certain other embodiments, the compounds of the invention modulate expression of dopamine receptors. In still other embodiments, the compounds of the invention modulate expression of mTOR or a Rho GTPase. In other embodiments, the compounds of the invention modulate expression of complement receptors (C3aR and C5aR). In still other embodiments, the compounds of the invention upregulate autophagy. In such embodiments, the upregulation of autophagy improves RPE health and reduces APOE deposits. In other embodiments, the compounds of the invention regulate AMPK. In still other embodiments, the compounds of the invention modulate RPE epithelial to mesenchymal transition or RPE dedifferentiation.

In particular embodiments, the compounds of the invention include, but are not limited to Aminocapropic acid, L-701,324, Vas2870, L-745,870 hydrochloride, Me-3,4-dephostatin, N-Methyl-1-deoxynojirimycin, L-750,667 trihydrochloride, (+)-MK-801 hydrogen maleate, Pempidine tartrate, (−)-Naproxen sodium, Raloxifene hydrochloride, SKF 83959 hydrobromide, L-687,384 hydrochloride, 7,7-Dimethyl-(5Z,8Z)-eicosadienoic acid, SP-600125, Ro 41-0960, Ancitabine hydrochloride, Risperidone, Telenzepine dihydrochloride, NO-711 hydrochloride, U-99194A maleate, S(+)-Raclopride L-tartrate, Pirenzepine dihydrochloride, Captopril, Thioperamide maleate, Alprenolol hydrochloride, Ritodrine hydrochloride, Putrescine dihydrochloride, 1-(2-Methoxyphenyl)piperazine hydrochloride, PAPP, U-69593, AG-1478, riluzole, Phentolamine mesylate, DBO-83, Formestane, Carbamazepine, 4-(2-Aminoethyl)benzenesulfonyl fluoride hydrochloride, Terbutaline hemisulfate, UK 14304, GR 113808, Leflunomide, Acetylthiocholine chloride, spermidine, 5-(N-Methyl-N-isobutyl)amiloride, ATPO, Acadenisine or Metformin, or combinations thereof.

In particular embodiments, the compounds of the invention are L-745,870; Riluzole, Aminocaproic Acid; Vas2870; Acadenisine; Metformin, or combinations thereof. In still other particular embodiments, the compound of the invention is metformin.

In additional embodiments when NOX4 inhibition is desired, the compounds or compositions of the invention include one or more siRNA molecules or one or more antibodies which inhibit NOX4. In certain embodiments, the compounds of compositions of the invention include one or more bioactive agents which results in the production of antibodies which inhibit NOX4.

In additional embodiments, the description provides the compounds as described herein including their enantiomers, diastereomers, solvates and polymorphs, including pharmaceutically acceptable salt forms thereof, e.g., acid and base salt forms.

Therapeutic Compositions

Pharmaceutical compositions comprising combinations of an effective amount of at least one compound as described herein, and one or more of the compounds otherwise described herein, all in effective amounts, in combination with a pharmaceutically effective amount of a carrier, additive or excipient, represents a further aspect of the present disclosure.

The present disclosure includes, where applicable, the compositions comprising the pharmaceutically acceptable salts, in particular, acid or base addition salts of compounds as described herein. The acids which are used to prepare the pharmaceutically acceptable acid addition salts of the aforementioned base compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] salts, among numerous others.

Pharmaceutically acceptable base addition salts may also be used to produce pharmaceutically acceptable salt forms of the compounds or derivatives according to the present disclosure. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (eg, calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of compounds according to the present disclosure in local ocular administration routes may also be used. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. Administration of compounds according to the present disclosure as eye drops, intravitreous injections, sub-tenon injections, and sub-retinal injections may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

In particular embodiments, the compounds described herein are administered by local ocular administration routes. In such embodiments, compounds according to the present disclosure are administered as ophthalmic pharmaceutical composition. Such ophthalmic pharmaceutical compositions are prepared in the form of eye drops, a mist, a frost, a foam, a cream, an ointment, or an emulsion for direct application to the eye. In particular embodiments, the compositions are prepared as aqueous eye drops. In such embodiments, the eye drops are monophasic. The concentration of compounds of the present invention contained in aqueous eye drops is generally, but without limitation, not less than 0.01 W/V %, preferably not less than 0.1 W/V %, more preferably not less than 0.5 W/V %, and generally not more than 20 W/V %, preferably not more than 10 W/V %, and more preferably not more than 5 W/V %. The amount of the compound of the present invention to be actually administered depends on the individual to be subjected to the treatment, and is preferably an amount optimized to achieve the desired treatment without accompanying marked side effects. The effective dose can be sufficiently determined by those of ordinary skill in the art.

The eye drop of the present invention can contain additives generally added to eye drops as necessary, as long as the characteristics of the present invention and the stability of the eye drop are not impaired. Examples of such additive include, but are not limited to, isotonicity agents such as sodium chloride, potassium chloride, glycerol, mannitol, sorbitol, boric acid, glucose, propylene glycol and the like; buffering agents such as phosphate buffer, acetate buffer, borate buffer, carbonate buffer, citrate buffer, tris buffer, glutamic acid, ε-aminocaproic acid and the like; preservatives such as benzalkonium chloride, benzethonium chloride, chlorhexidine gluconate, chlorobutanol, benzyl alcohol, sodium dehydroacetate, paraoxybenzoate esters, sodium edetate, boric acid and the like; stabilizers such as sodium bisulfite, sodium thiosulfate, sodium edetate, sodium citrate, ascorbic acid, dibutylhydroxytoluene and the like; water-soluble cellulose derivatives such as methylcellulose, hydroxyethylcellulose, hydroxypropylmethylcellulose, carboxymethylcellulose and the like; thickeners such as sodium chondroitin sulfate, sodium hyaluronate, carboxyvinyl polymer, polyvinyl alcohol, polyvinylpyrrolidone, macrogol and the like; pH adjusters such as hydrochloric acid, sodium hydroxide, phosphoric acid, acetic acid and the like; and the like. In particular embodiments, eye drops comprising compounds of the present invention may further contain one or more other ingredients which can be contained in artificial tears, i.e., aminoethylsulfonic acid, sodium chondroitin sulfate, potassium L-aspartate, magnesium L-aspartate, potassium magnesium L-aspartate (equimolar mixture), sodium hydrogen carbonate, sodium carbonate, potassium chloride, calcium chloride, sodium chloride, sodium hydrogen phosphate, sodium dihydrogen phosphate, potassium dihydrogen phosphate, exsiccated sodium carbonate, magnesium sulfate, polyvinylalcohol, polyvinylpyrrolidone, hydroxyethylcellulose, hydroxypropylmethylcellulose, glucose, and methylcellulose. While the amount of these additives to be added varies depending on the kind, use and the like of the additive to be added, they only need to be added at a concentration capable of achieving the object of the additive.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions as described herein may be administered orally, parenterally, by inhalation spray, topically, intraocularly, to the ocular surface, rectally, nasally, buccally, vaginally or via an implanted reservoir. The term “parenteral” as used herein includes subcutaneous, intravenous, intramuscular, intra-articular, intra-synovial, intra-vitreous, sub-retinal, retinal, sun-tenon, intrasternal, intrathecal, intrahepatic, intralesional and intracranial injection or infusion techniques. Preferably, the compositions are administered by local ocular administration, orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added. In certain embodiments, pharmaceutical compositions for oral administration include formulations which aid in delivering the compound across the blood-retina barrier.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions as described herein may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. In certain preferred aspects of the disclosure, the compounds may be coated onto a stent which is to be surgically implanted into a patient in order to inhibit or reduce the likelihood of occlusion occurring in the stent in the patient.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. In certain embodiments, pharmaceutical compositions for ophthalmic or local ocular use include lipophiliccally modified compositions and transplantable carriers.

The pharmaceutical compositions as described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition as described herein that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one other compound according to the present disclosure.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure including pharmaceutically acceptable salts, solvates or polymorphs, thereof optionally in a pharmaceutically acceptable carrier or diluent, either alone, or in combination with other known therapeutic agents as otherwise identified herein.

These compounds can be administered by any appropriate route, for example, orally, parenterally, intravenously, intradermally, subcutaneously, or topically, including transdermally, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents, including epidermal growth factor receptor inhibitors, EPO and darbapoietin alfa, among others. In certain preferred aspects of the disclosure, one or more compounds according to the present disclosure are coadministered with another bioactive agent, or a wound healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

Therapeutic Methods

In an additional aspect, the description provides therapeutic compositions comprising an effective amount of a compound as described herein or salt form thereof, and a pharmaceutically acceptable carrier. The therapeutic compositions can be used for treating or ameliorating ophthalmic disease states or conditions in a patient or subject, for example, an animal such as a human. The therapeutic compositions can be used for treating or ameliorating retinal disorders or conditions in a patient or subject, for example, an animal such as a human.

The terms “treat”, “treating”, and “treatment”, etc., as used herein, refer to any action providing a benefit to a patient for which the present compounds may be administered, including the treatment of an ophthalmic disease state or condition. The description provides therapeutic compositions as described herein for treating ophthalmic diseases, such as age-related) macular degeneration, macular dystrophies such as Stargardt's and Stargardt's-like disease, Best disease (vitelliform macular dystrophy), and adult vitelliform dystrophy or subtypes of retinitis pigmentosa. In certain embodiments, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating an ophthalmic disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In additional embodiments, the description provides methods for treating retinal degradation in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating a symptom of retinal degradation in the subject.

In additional embodiments, the description provides methods for restoring retinal pigment epithelium cells in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for restoring retinal pigment epithelium cells in the subject.

In additional embodiments, the description provides methods for treating macular degeneration in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating a symptom of macular degeneration in the subject. In specific embodiments, the macular degeneration is age-related macular degeneration. In other embodiments, the macular degeneration is atrophic, neovascular or exudative macular degeneration. In other embodiments, the macular degeneration is early stage macular degeneration, intermediate stage macular degeneration, or advanced stage macular degeneration. In a particular embodiment, the description provides methods for treating early stage macular degeneration in a subject or a patient, e.g., an animal such as a human, comprising administering to a subject in need thereof a composition comprising an effective amount, e.g., a therapeutically effective amount, of metformin or salt form thereof, and a pharmaceutically acceptable excipient, carrier, adjuvant, another bioactive agent or combination thereof, wherein the composition is effective for treating or ameliorating a symptom of early stage macular degeneration in the subject.

In another embodiment, the present disclosure is directed to a method of treating or ameliorating an ophthalmic disease in a human patient in need thereof, the method comprising administering to a patient in need an effective amount of a compound according to the present disclosure, optionally in combination with another bioactive agent.

In another embodiment, the description provides methods for treating Stargardt's disease or a Stargardt's-like disease, in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating a symptom of Stargardt's disease or a Stargardt's-like disease, in the subject. In particular embodiments, the methods for treating Stargardt's disease or a Stargardt's-like disease comprises administration of an effective amount of metformin or a salt thereof.

The term “bioactive agent” is used to describe an agent, other than a compound according to the present disclosure, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered.

The term “pharmaceutically acceptable salt” is used throughout the specification to describe, where applicable, a salt form of one or more of the compounds described herein which are presented to increase the solubility of the compound in the gastic juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present disclosure.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

Kits

In an additional aspect, the description provides kits which, when used by the medical practitioner, can simplify the administration of appropriate amounts of the compounds of the invention or pharmaceutically acceptable salts, solvates or hydrate thereof to a patient or cell.

A typical kit of the invention comprises one or more units dosage forms of a compound of the invention or pharmaceutically acceptable salts, solvates or hydrates thereof, and instructions for administration to a subject or cell. A typical kit of the invention could also, or alternatively, contain a bulk amount of a compound of the invention or pharmaceutically acceptable salts, solvates or hydrates thereof.

Kits of the invention can further comprise devices that are used to administer a compounds of the invention or pharmaceutically acceptable salts, solvates or hydrates thereof, and instructions for administration to a subject or cell. Examples of such devices include, but are not limited to, intravenous cannulation devices, syringes, drip bags, patches, topical gels, pumps, containers that provide protection from photodegradation, autoinjectors, eye droppers, and inhalers.

In a particular embodiment, the kits of the invention comprise a solution comprising a compound of the invention or pharmaceutically acceptable salts, solvates or hydrates thereof, an eye dropper and instructions for administration of the solution directly to the eye of the subject. In certain such embodiments, the solution is provided in a container comprising a dropper tip which can dispense drops directly without an additional eye dropper.

Kits of the invention can further comprise pharmaceutically acceptable vehicles that can be used to administer one or more compounds of the invention as active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

### Example 1—Complement Competent Human Serum (CC-HS) Induces AMD-Like Cellular Endophenotypes in Mature iPSC-RPE

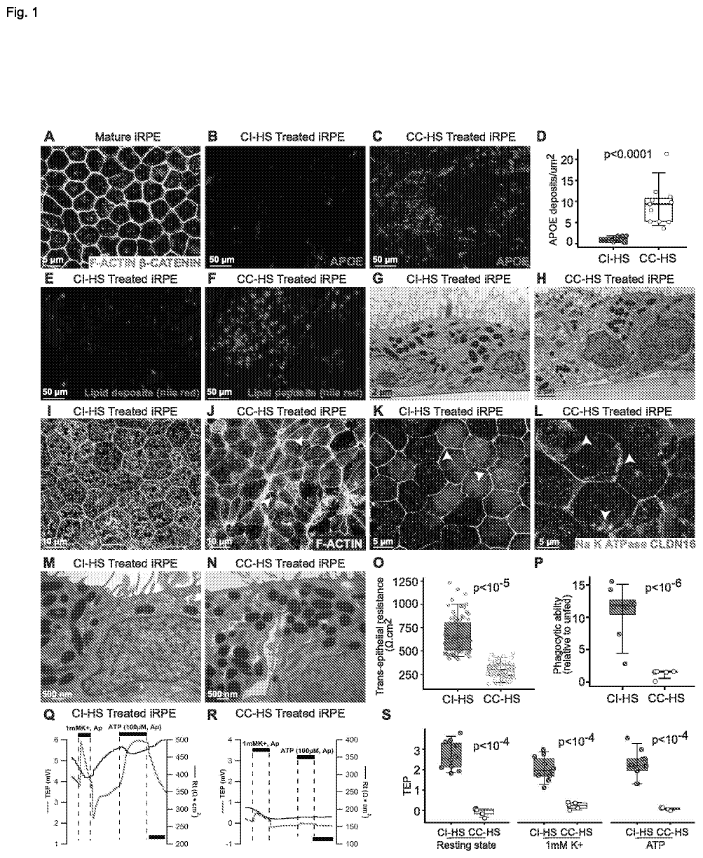

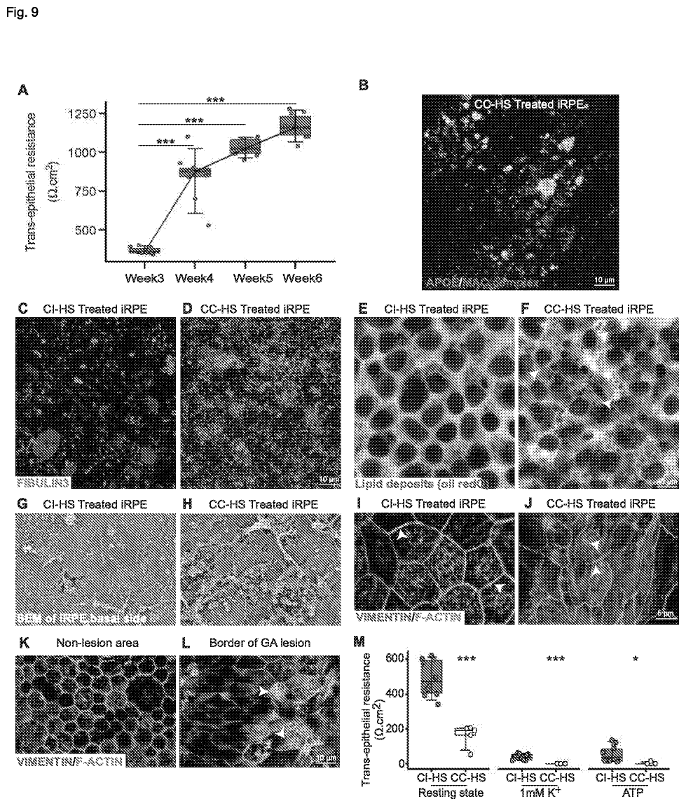

iPSCs derived from five different healthy individuals were used for this analysis. iPSCs were differentiated into mature RPE cells using previously published protocol (May-Simera et al., 2018, Sharma et al., 2019). Maturity of iRPE cells was confirmed by the presence of β-catenin on the cell membrane, (May-Simera et al., 2018, FIG. 1A) and by progressively increasing trans-epithelial resistance (TER) of the monolayer starting week 3 of culture (p<2×10⁻¹⁶, week 3 to weeks 4-6; FIG. 9A). Consistent with published reports for primary RPE cells (Johnson et al., 2011; Pilgrim et al., 2017), a five-fold increase in APOE positive sub-iRPE deposits was observed in CC-HS treated iRPE as compared to CI-HS treated cells (p<0.0001; FIGS. 1B-D). Similar to drusen deposits seen in AMD eyes (Mullins et al., 2000 FASEB J) APOE positive deposits co-stained with an anti-membrane attack complex (MAC) antibody (FIG. 9B). CC-HS treated iRPE also expressed higher levels of drusen marker Fibulin 3 (Marmostein et al., 2002; FIG. 9C, D), increased sub-RPE staining for neutral lipid deposits (Nile red) and increased intracellular staining for triglycerides and esterified cholesterol deposits (Oil red 0) (Pilgrim et al., 2017; FIGS. 1E, F and 9E, F). The presence of intracellular lipid deposits was further confirmed by transmission electron microscopy (TEM) of CC-HS treated iRPE cells (yellow arrowheads in FIG. 1H). TEM and scanning electron microscopy (SEM) also verified the presence of basal-laminar deposits with typical dome-shaped appearance in CC-HS treated samples (red arrowheads FIGS. 1G, H and 9G, H). Together, these findings support the claim that CC-HS treatment induces several characteristic disease phenotypes of AMD in iRPE cells. Thus, providing an in vitro model to investigate RPE cell-autonomous pathways involved in AMD pathogenesis and to discover drugs that could intervene at an earlier disease stage.

TEM of CC-HS treated cells also revealed disintegrated junctional complexes between neighboring RPE. cells (arrowhead in FIGS. 1 I, J). To further investigate the integrity of tight junctions between neighboring RPE cells in CC-HS treated samples, samples were stained for tight junctions and the actin cytoskeleton markers. CLDN16—a critical RPE tight junction protein (Wang et al., 2010) was often missing from cell borders and localized intracellularly in CC-HS treated samples (arrowheads in FIGS. 1 K, L). F-ACTIN staining displayed intracellular stress fibers in CC-HS treated iRPE cells that failed to retain their characteristic hexagonal morphology (arrowheads in FIGS. 1M, N). VIMENTIN immunostaining further confirmed dedifferentiation of CC-HS treated iRPE cells with VIMENTIN missing from cell membranes and present without any structure in the cytoplasm of enlarged, stretched out cells (Tamiya et al., 2010; FIGS. 9I, J). Loss of epithelial phenotype has been reported in patient eyes using Optical Coherence Tomography (Curcio et al 2017). To check if this previously reported loss of RPE epithelia phenotype is consistent with the dedifferentiation phenotype seen in the CC-HS iRPE model, RPE-flatmounts from cadaver AMD eyes were immunostained for VIMENTIN and F-ACTIN (FIGS. 9K, L).

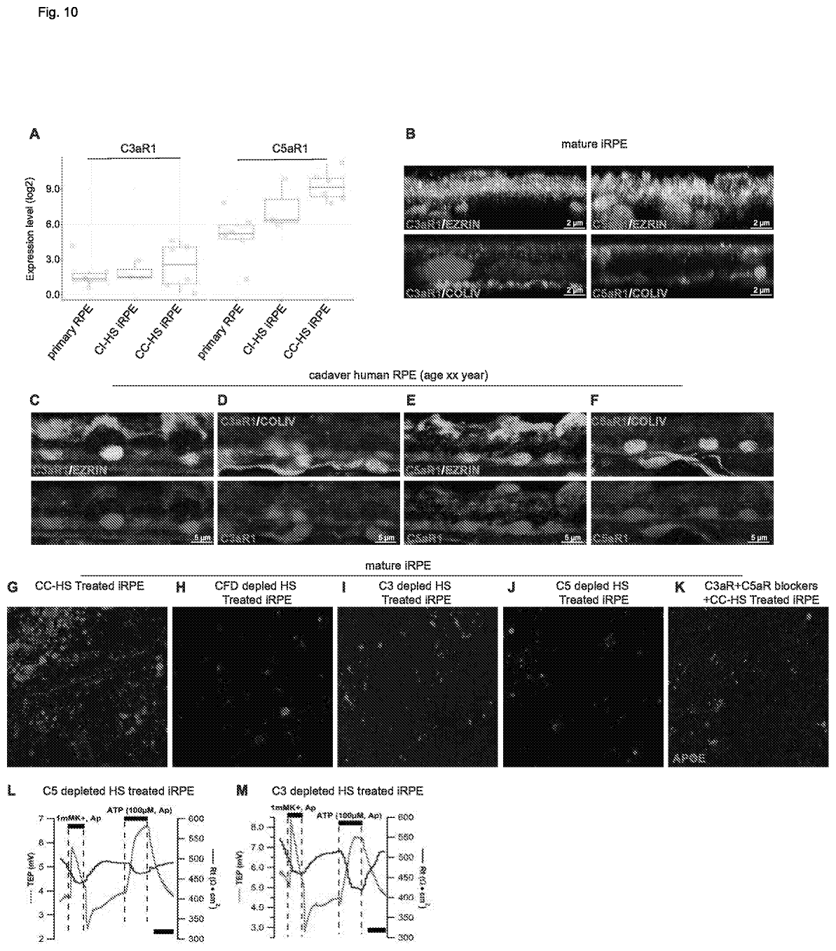

Dedifferentiation of RPE cells at the borders of GA lesion was confirmed by F-ACTIN staining showing the loss of typical hexagonal morphology and increased VIMENTIN immunostaining present in the cytoplasm without any proper structure (FIG. 9K, L), confirming in vitro observations. Dedifferentiation of CC-HS treated iRPE cells resulted in a 3-fold reduction (p<10⁻⁵) in the TER, compared to CI-HS treated iRPE (FIG. 10). It also resulted in a loss of functional maturity of CC-HS treated cells as confirmed by a 6-fold reduced (p<10⁻⁶) ability to phagocytose photoreceptor outer segments (POS) (FIG. 1P). Additionally, CC-HS treated cells as compared to CI-HS treated cells lost their polarized status, as demonstrated by a reduced steady-state trans-epithelial potential (TEP) (4 mV v/s 0.25 mV, p<10⁻⁴), lower hyperpolarization response to a physiological stimulus of reducing apical K+ concentration from 5 to 1 mM (2 mV v/s 0.5 mV, p<0.0001), and a negligible depolarization response to an apical ATP stimulus (p<0.03; FIGS. 1Q, R; 9M). Overall the results showed that CC-HS treatment induces several hallmark features seen in AMD RPE; most notably, the formation of APOE and lipid-containing sub-cellular deposits. This work extends the previous knowledge that CC-HS treatment and sub-RPE deposits are associated with degeneration of epithelial phenotype with loss of apical-basal polarity and functional maturity leading to dedifferentiation of cells, a phenotype that is thought to lead to advanced disease stages.

### Example 2—CC-HS Triggered AMD Disease Phenotypes are Induced Through C3aR1 and C5aR1 Signaling

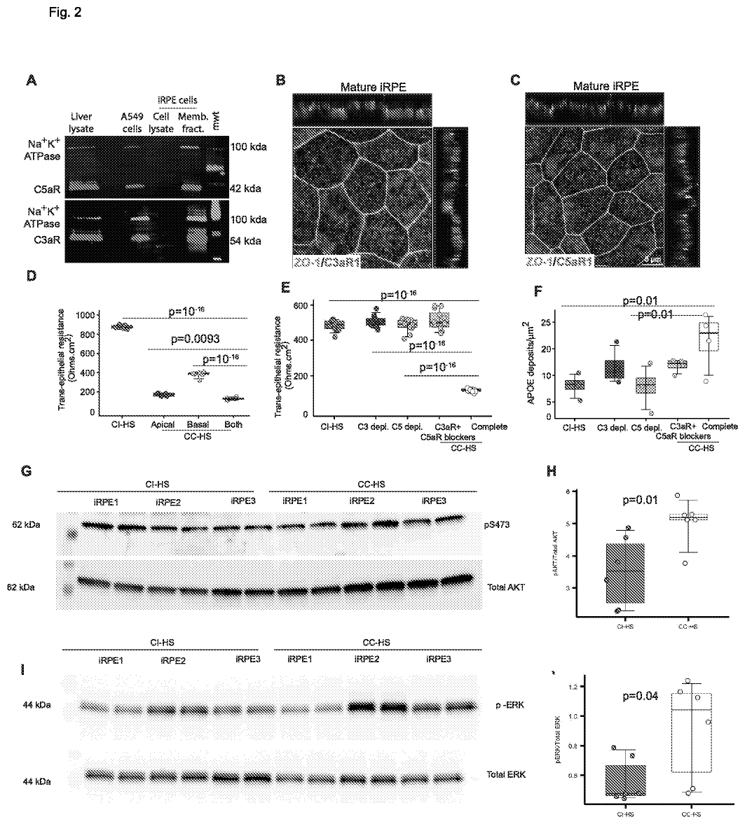

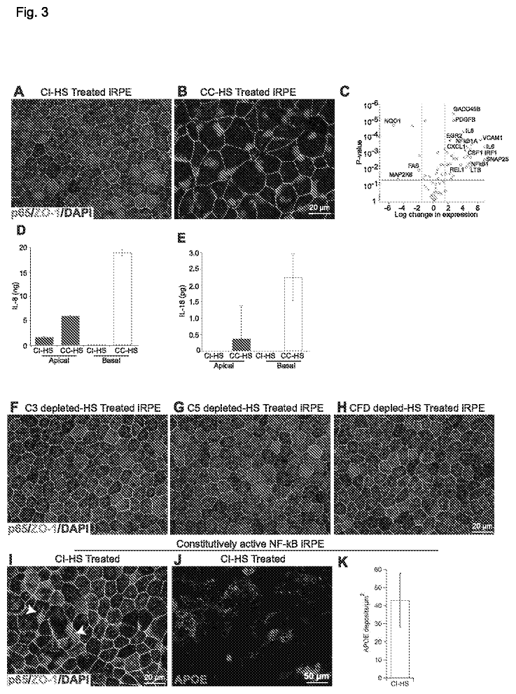

It was hypothesized that CC-HS triggered AMD cellular phenotypes in iRPE cells are generated by the anaphylatoxin arm of the complement pathway via C3a-C3aR1 and C5a-05aR1 signaling induced intracellular inflammation (Fernandez-Godino and Pierce 2018). RNAseq confirmed the expression of both receptors in iRPE cells with ˜30× higher expression in of C5aR1 as compared to C3aR1 (FIG. 10A). Furthermore, the expression of both receptors increases with CC-HS treatment. Western blot confirmed C3aR1 and C5aR1 receptors localization in the membrane fraction of iRPE cells (FIG. 2A). Their colocalization with the apical membrane marker EZRIN, but not with the basal membrane marker COLLAGEN IV suggests that the receptors for the two complement proteins, C3a and C5a, are predominantly apically located (FIGS. 2B, C, 10B). To verify that CC-HS activates C3aR and C5aR signaling in iRPE cells, samples were checked for phosphorylation of AKT and ERK1/2, the two key kinases downstream of C3aR1 and C5aR1 receptors (Hajishengallis and Lambris 2010; Zhu et al 2015 Mol Vis; Busch et al 2017 Front. In Immu.). Western blot for CC-HS treated samples across three donor iRPE samples showed 2-4× increased levels of pAKT (p<0.01) and pERK1/2 (p<0.01) in CC-HS treated cells as compared to CI-HS treated cells (FIGS. 2D-G). Furthermore, consistent with the predominant apical localization of C3aR1 and C5aR1, apical only treatment of CC-HS caused a 4.5-5×TER drop (p<10⁻¹⁶), similar to the combined apical/basal treatment of CC-HS. In contrast, basal only treatment of CC-HS resulted only in a 2×TER drop (p<10⁻¹⁶) (FIG. 2H). To further dissect the role of C5aR1 and C3aR1 signaling in inducing AMD cellular phenotypes in iRPE cells, a depleted sera and receptor blocker strategy was employed. Treatment of iRPE cells with sera depleted in C3 (p<0.01) or C5 (p<0.01) proteins, or the concurrent use of blockers for C3aR1 (compstatin, 10 μM) and C5aR1 (PMX053, 10 μM) in CC-HS serum (p<0.01) resulted in 2× lower sub-RPE APOE deposits as compared to CC-HS plus vehicle treatment (FIGS. 2I, 10C-G). Depletion in complement factor D, an upstream regulator of C3a and C5a formation, also led to lower sub-RPE APOE deposits as compared to complete CC-HS medium (Sharma and Ward 2011; Figures S2C, D). Similarly, as compared to CC-HS treated samples, a 5× higher iRPE monolayer TER was noticed in samples treated with sera depleted in C3 (p<10⁻¹⁶) or C5 (p<10⁻¹⁶) or with the use of blockers for C3aR1 and C5aR1 receptors (compstatin, 10 μM and PMX053, 10 μM respectively; p<10⁻¹⁶) (FIG. 2J). Furthermore, no changes in electrophysiological properties of iRPE monolayers were seen in samples treated with sera depleted in C3 and C5 proteins (compare FIG. 1Q, R with 10 H, I). In summary, the data suggests that stimulation of C3aR1 or C5aR1 complement receptors occurring predominantly through the apical surface of RPE cells is required for triggering AMD disease phenotype in iRPE cells.

### Example 3—C3aR1 and C5aR1 Induced subRPE Deposits are Mediated by Overactivation of NF-KB and Downregulation of Autophagy Pathways

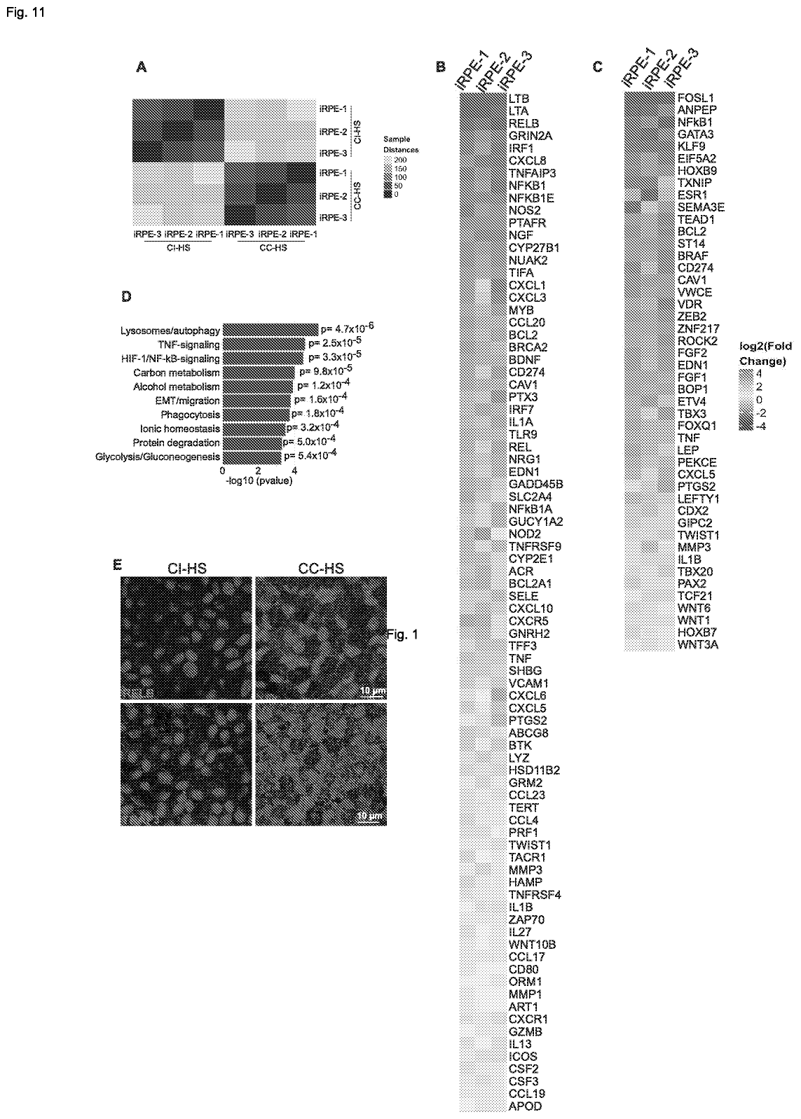

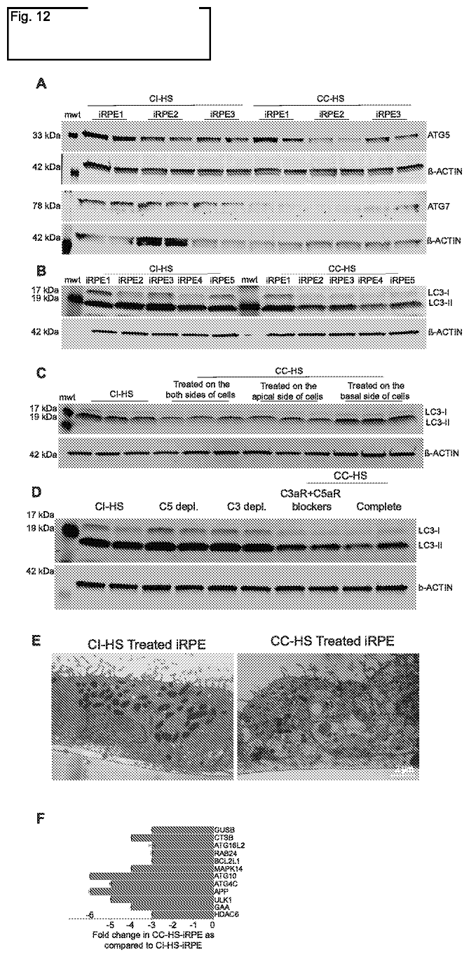

RNAseq analysis of CI-HS and CC-HS treated iRPE cells revealed dramatically different global gene expression pattern induced by CC-HS treatment (FIG. 11A). Consistent with the effect of anaphylatoxin complement (C3a, C5a) in immune cells, autophagy (p<10⁻⁶) and TNF/NF-KB (p<10⁻⁵) pathways were the most changed by CC-HS treatment of iRPE cells (Freeley et al., 2016; Kumar 2019 Int Rev of Immu; Nguyen et al., 2018; FIG. 11B, C, D). This led us to hypothesize that C3aR1 and C5aR1 signaling in iRPE cells is working through these two pathways. CC-HS treatment indeed caused p65 (RELA) subunit of NF-KB to translocate to the nucleus, suggesting its activation (Rayet and Gelinas 1999, Oncogene; FIG. 3A, B). Nuclear translocation of p65 led to 4-6× increased expression of NF-KB target genes (Tilborghs et al., 2017), as confirmed by RNAseq (FIG. 11B, p<10⁻⁵), qRT-PCR-based validation of selected target genes (e.g. IL-6, IL-8, GADD45B, EGR2, NFκB1A, REL1, NFκB1, SNAP25; FIG. 3C, p<10⁻¹ to p<10⁻⁶), and immunostaining for two NF-κB target genes RELB and TRAF3 (FIG. 11B-D). Furthermore, CC-HS treatment doubled the secretion of inflammatory cytokines of the NK-κB pathway, IL-8 (FIG. 3D, apical p<0.01, basal p<10⁻⁵) and IL-18 (FIG. 11E, apical p<0.005, basal p<0.005). Lacking nuclear translocation of p65 in iRPE cells treated with sera depleted with C3 or C5 proteins further confirmed the role of anaphylatoxin complement in direct activation NF-KB pathway in iRPE cells (FIG. 3E-G). To determine if NF-κB activation directly led to the formation of sub-RPE APOE deposits, iRPE cells derived from a patient with E391X mutation in gene NEMO were used, a negative regulator of NF-κB signaling (Zilberman-Rudenko et al., 2016). Consistent with the literature, nuclear translocation of p65 was seen in mutant iRPE cells even under CI-HS treatment conditions (FIG. 3H). Furthermore, 5-6× higher (p<0.001) sub-RPE APOE deposits were seen in mutant iRPE cells under CI-HS treatment conditions (FIGS. 3I, J). Overall, these results demonstrate that the anaphylatoxin complement triggered AMD disease phenotypes in iRPE cells are likely mediated through the activation of NF-kB pathway.

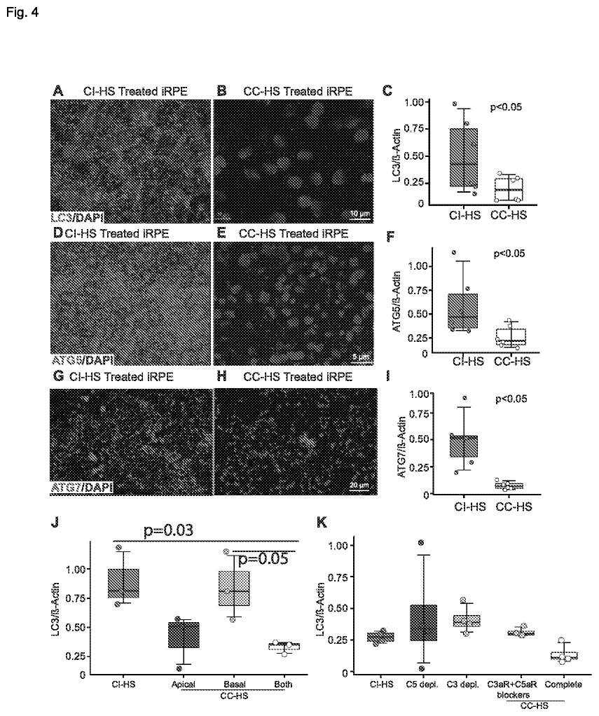

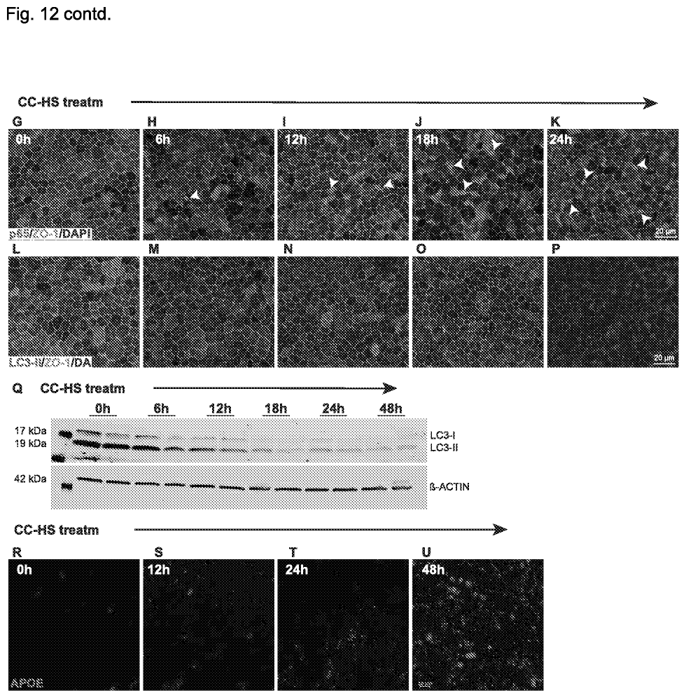

RNAseq analysis also revealed statistically significant (p<10⁻⁶) defects in autophagy pathway in CC-HS treated cells (FIG. 11C), which is supported by literature link between AMD and autophagy dysregulation in the RPE (Sinha et al., 2016; Golestaneh et al., 2017). This prompted us to investigate the role of autophagy in CC-HS induced AMD cellular endophenotypes in iRPE cells Immunostaining and Western blot analyses revealed that genes integral for autophagy regulation, ATG5, ATG7, and LC3-II, were all 3-4× downregulated (p<0.005) in CC-HS treated iRPE cells, compared to CI-HS treated iRPE cells (FIGS. 4A-I; 12A, B). These results were further corroborated by qRT-PCR, which showed a 4-16 fold downregulated (p<0.01-10⁻³) expression of crucial autophagy pathway genes (e.g. ATG3, ATG12, ATG4B, ATG4D, BCL2, LAMP1, SQSTM1, MAP1LC3A, MAP1LC3B) in CC-HS treated iRPE cells, as compared to CI-HS iRPE cells (FIG. 12F). Reduced expression of key autophagy genes in CC-HS treated cells suggested reduced autophagy flux, which was confirmed by increased accumulation of autophagosomes in CC-HS treated iRPE cells (arrowheads, FIG. 12E).

Consistent with the predominant apical localization of C3aR1 and C5aR1, it was demonstrated that treatment of CC-HS on the apical side of the iRPE was sufficient to trigger downregulation of LC3-II, a major marker for autophagy, in cells (FIG. 4J, 12C; p=ns between both sides CC-HS and apical only CC-HS treatment). Furthermore, unlike CC-HS treated cells, iRPE cells treated with CC-HS sera depleted in C3 or C5 proteins were incapable of inducing autophagy downregulation and behaved similar to CI-HS serum (FIG. 4K, 12D, p<0.05 CI-HS vs CC-HS; p=ns CI-HS vs C5 depl. Or C3 dept CC-HS). Similarly, iRPE cells treated with CC-HS coupled with C3aR1 and C5aR1 blockers behaved similar to CI-HS treated samples and didn't show statistically significant reduction in LC3 levels (p=ns CI-HS vs C3aR+C5aR blockers+CC-HS; FIG. 4K, 12D). Overall, these results confirm that the anaphylatoxin complement signaling C3aR1 and C5aR1 inhibits autophagy in CC-HS treated iRPE cells.

To further understand the sequence of events leading to the formation of sub-RPE drusen deposits, a temporal analysis of NF-κB activation and autophagy downregulation after the addition of CC-HS on to iRPE cells was performed. Within 6 hours of CC-HS addition, nuclear translocation of p65 was evident in just a few cells and over 24 hours this translocation was seen in most of the cells (FIGS. 12g-k). Similarly, LC3-II downregulation could be seen immediately within 6 hours of CC-HS treatment reaching maximum levels by 24 hours (FIGS. 12l-q). In contrast, a clear increase in APOE deposits was seen only at the 48 hour time point (FIGS. 12r-u), suggesting that NF-KB upregulation and autophagy downregulation precede the formation of APOE deposits in CC-HS treated iRPE cells.

Previously, NF-kB signaling and autophagy have been linked to STAT3 activity (Jonchere et al., 2015). Reduced STAT3 transcriptional activity is thought to result in autophagy downregulation (Jonchere et al., 2015). To check if STAT3 activity was changed in CC-HS treated samples, STAT3 phosphorylation in CC-HS and CI-HS treated samples were compared. Phosphorylation of STAT3 at tyrosine residue 705 was 5-6× downregulated (p<0.001), suggesting reduced transcription activity of STAT3. To confirm if reduced STAT3 activity in iRPE cells directly leads to subRPE APOE deposit formation, RPE cells from a patient with Job's syndrome were generated. Because of a DNA binding mutation, STAT3 is transcriptionally inactive in these cells. Consistent with previous data, 10-12× higher APOE positive subRPE deposits are seen in STAT3 mutant cells as compared to control cells even under CI-US treatment conditions. This suggests that STAT3 downregulation downstream of NF-kB over-activation also contributes to APOE deposit formation.

### Example 4—High Throughput Screen to Discover iRPE Cytoprotective Drugs

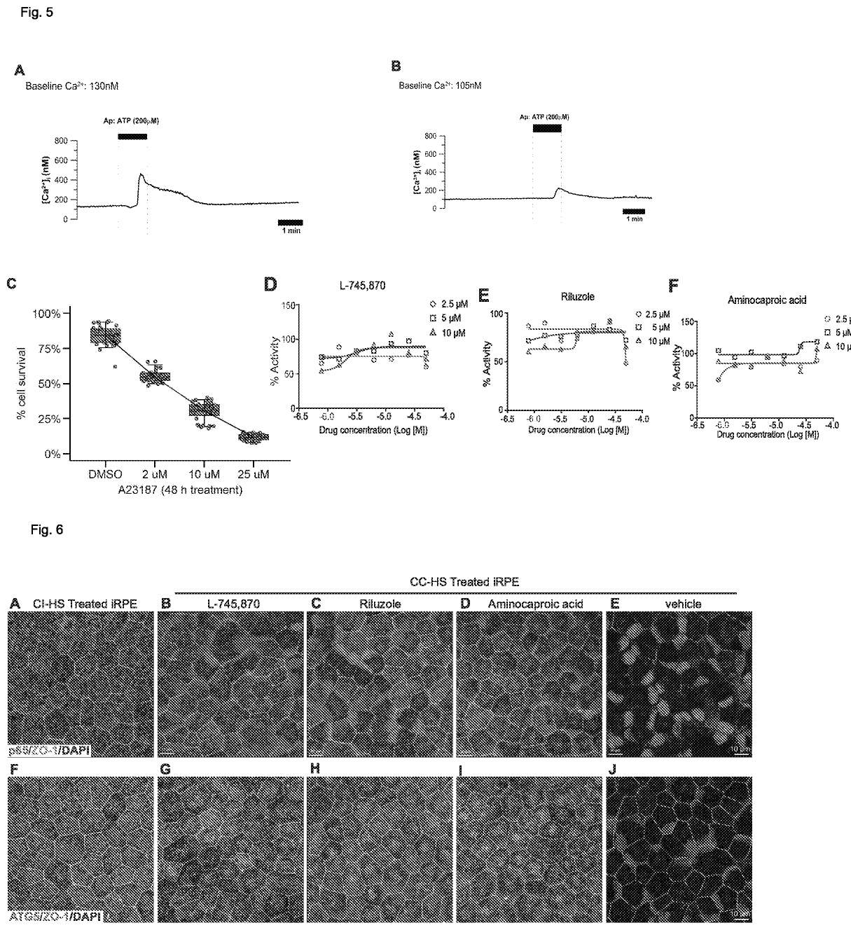

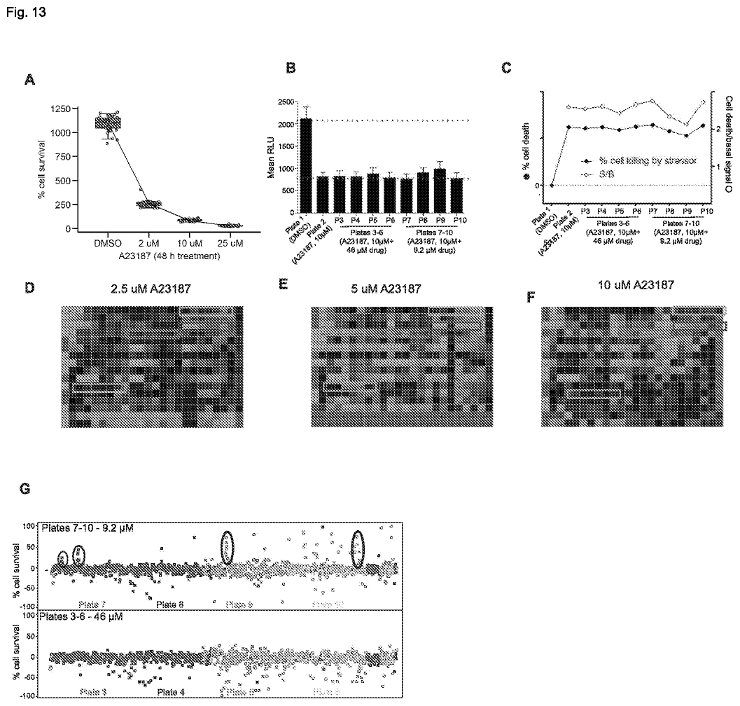

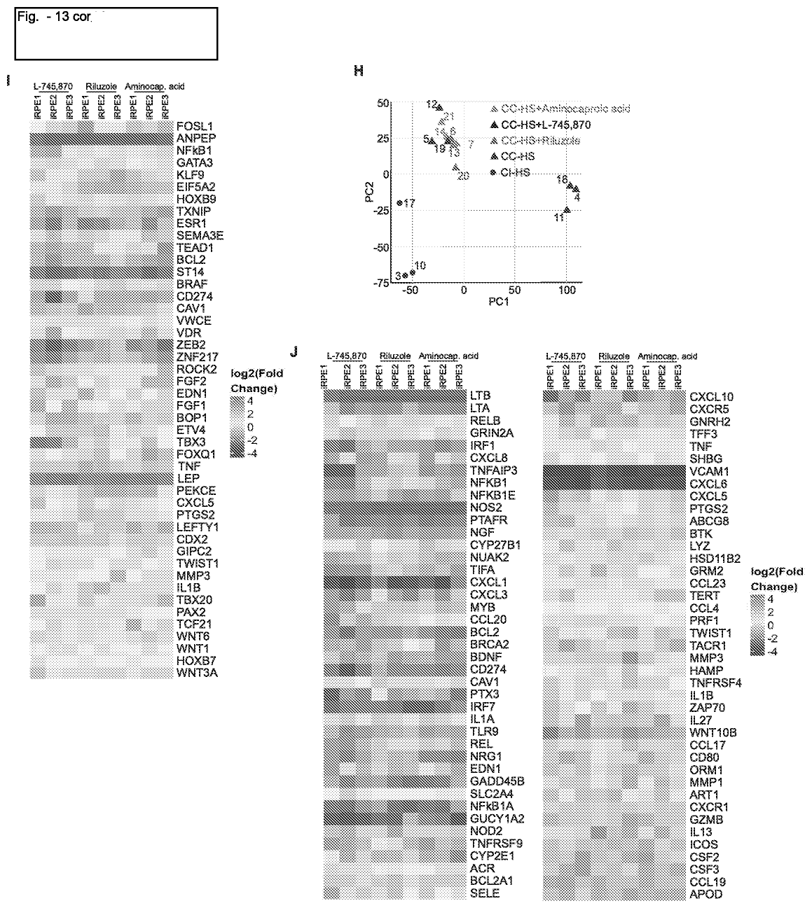

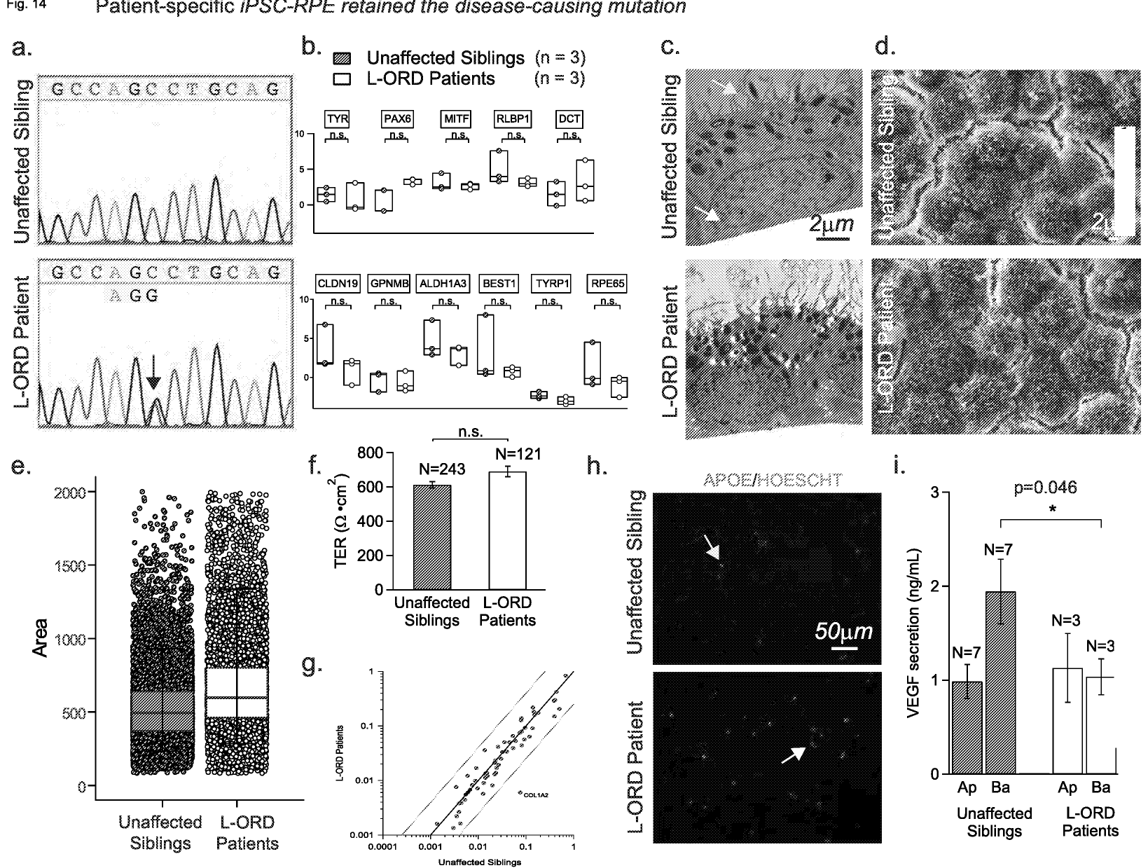

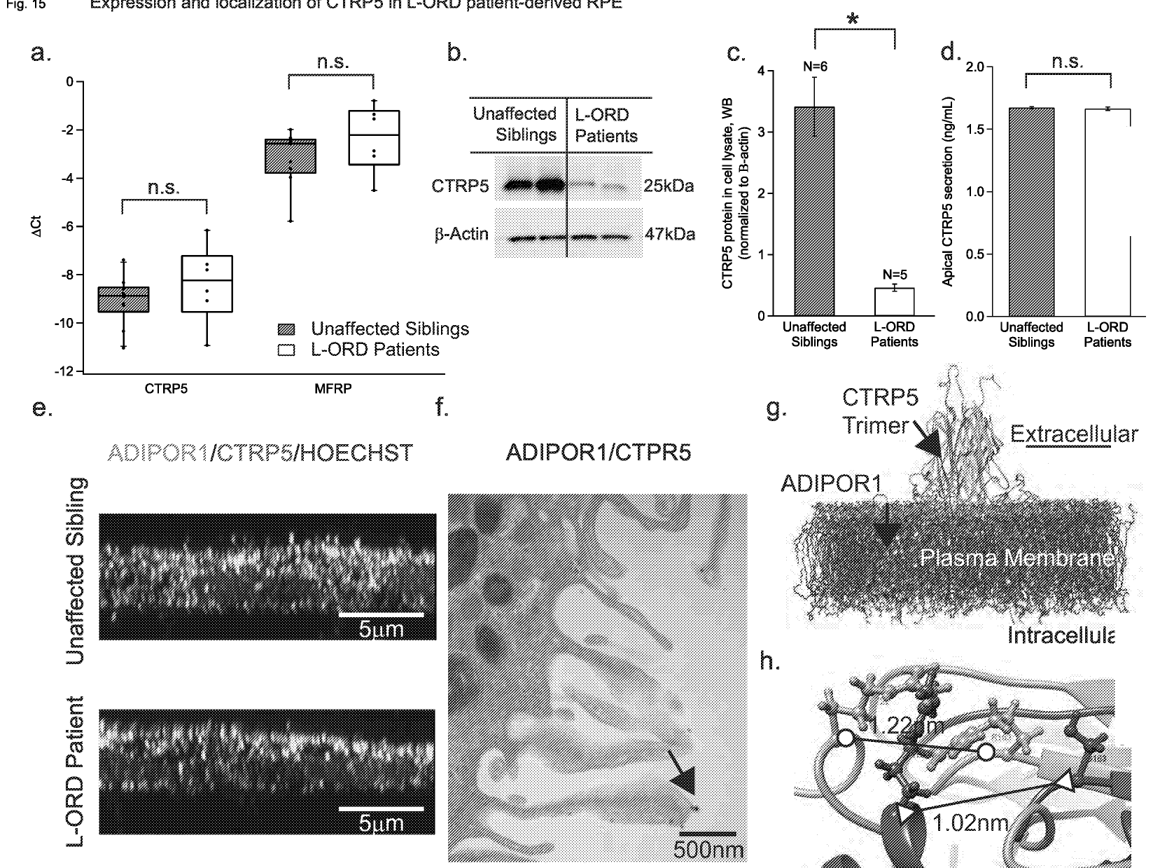

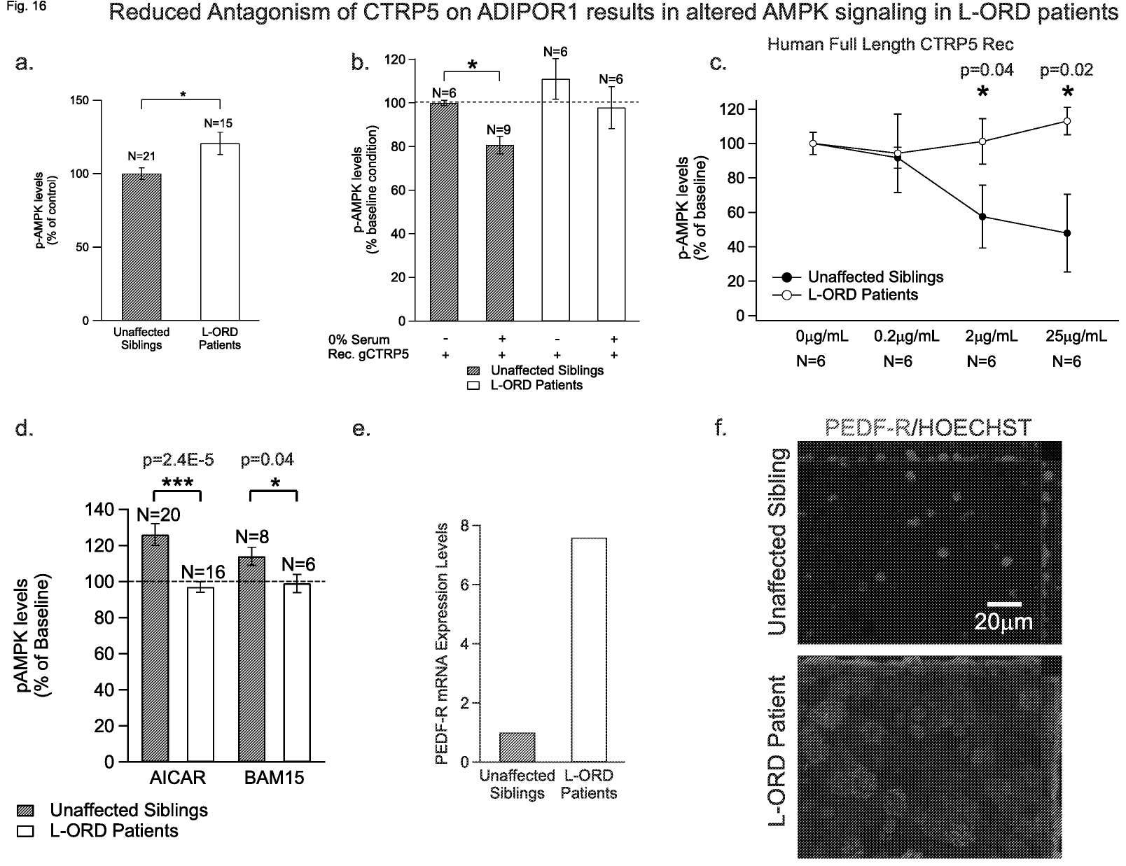

RNAseq revealed a highly complex response triggered in CC-HS treatment of iRPE—with multiple pathways including TNF/NF-KB, autophagy, carbohydrate metabolism, protein degradation, ionic homeostasis and the epithelial phenotype affected in cells (FIG. 11B). It was hypothesized that the loss of epithelial phenotype/RPE dedifferentiation is a key AMD cellular phenotype and easier to set up for a high throughput screen. Drugs that would suppress RPE dedifferentiation and recover the epithelial phenotype in cells might also work to rescue additional AMD cellular endophenotypes. Drug screen was designed using a calcium-ionophore A23187 instead of CC-HS serum, for three reasons: 1) CC-HS lost activity in liquid handler tubing used for medium change, likely because active complement proteins were absorbed on the walls of liquid handler tubes; 2) Similar to A23187, CC-HS treatment of iRPE also led to a defect in intracellular calcium homeostasis. Although the baseline calcium levels in CI-HS and CC-HS treated cells were similar (100-120 nM; FIGS. 5A-C), ATP stimulation that leads to an activation of intracellular calcium stores and increase intracellular calcium was significantly dampened in CC-HS treated cells (80 nM increase) as compared to CI-HS treated cells that showed an increase of 200 nM (FIGS. 5A-C, p<0.05); Similar to CC-HS treatment, A23187 treatment led to iRPE cell death (FIGS. 5D, 13A).

At 96 hours all three concentrations of A23187 (2.5, 10, 25 μM) were completely toxic to cells, but at 48 hours 2.5 μM caused 50% cell death, and 10 μM caused 70% cell death (FIGS. 5D, S6A). 10 μM concentration of A23187 was selected for the drug screening, because it provided a bigger margin for cell death rescue. A commercially available library of pharmaceutically active compounds (LOPAC) with 1280 drugs was used for the screen at two different concentrations 9.2 μM and 46 μM. Drug treatment along with the stressor, 10 μM A23187 were added on to iRPE cells matured in 384-well plates and cell death was scored 48 hours later by ATP release using the CellTitrGlo assay. Comparable relative mean intensity of signal across all of the assay plates containing A23187 confirmed consistency and reproducibility of the screen (FIG. 13B). Normalized cell death signal showed that plates 7-10 with the lower drug concentration (9.2 μM) had reduced cell death as compared to plates with the higher drug concentration (46 μM) (FIG. 13C). In fact, at 46 μM most drugs were cytotoxic, whereas at 9.2 μM 45 of the drugs showed improved (more than 40%) cell survival in A23187 treated cells (FIG. 13G).

A closer analysis of the drug data revealed potential artifacts in some of the 384-well lanes may have contributed to false positive signals (circled in FIG. 13G). To distinguish false positives from the real signal, a follow up screen on all of the 45 drugs using iRPE derived from two different iPSC lines was performed. The follow up screen was performed at three different A23187 concentrations (2.5, 5, 10 μM) and with seven different concentrations of drugs ranging from 10 nM-1 mM (FIG. 13D-F). Only two drugs (L, 745,870 and aminocaproic acid (ACA); FIG. 5E, F) exhibited reproducible cytoprotective activity across the two iRPE samples, confirming the initial observation of false-positive effects across several 384-well lanes (FIG. 13G). Overall, the HTS screen provided two drugs for testing in the in vitro AMD model.

### Example 5—L456,780 and Aminocaproic Acid Reversed CC-HS Induced NF-kB Activation and Autophagy Suppression in iRPE Cells

Based on the seven-point dose response curve (FIGS. 5D-F), the IC50 dose for both the drugs was selected (6 μM for L456,780 and 30 μM for aminocaproic acid (ACA)) to co-treat iRPE cells along with CC-HS Immunostaining for p65 revealed reduced nuclear localization in iRPE co-treated with CC-HS and L745,870 or CC-HS and aminocaproic acid, as compared to CC-HS and vehicle (DMSO) co-treated samples (FIGS. 6A-E). RNAseq further confirmed that co-treatment of iRPE with CC-HS and L, 745,870 or aminocaproic acid reversed gene expression changes induced by CC-HS treatment (FIG. 13H, I) and reduced (up to 16-fold) the expression of NF-KB pathway genes as compared to CC-HS and DMSO co-treated samples (FIG. 13I). Consistently, autophagy genes that were downregulated in CC-HS treated iRPE were upregulated in iRPE co-treated with CC-HS and either of the two drugs (FIGS. 6F-J; 13J, as confirmed by immunostaining for ATG5 (FIGS. 6F-J), Overall, these results demonstrated that both cytoprotective drugs discovered in the HTS were able to reverse the effects of CC-HS treatment on iRPE cells by suppressing NF-KB pathway and upregulating autophagy. This prompted us to further test the effect of these drugs on RPE-epithelial phenotype, functions, and AMD cellular endophenotypes.

### Example 6—Restoration of iRPE Epithelial Phenotype In Vitro in CC-HS Treated Cells and In Vivo in a Rat Model

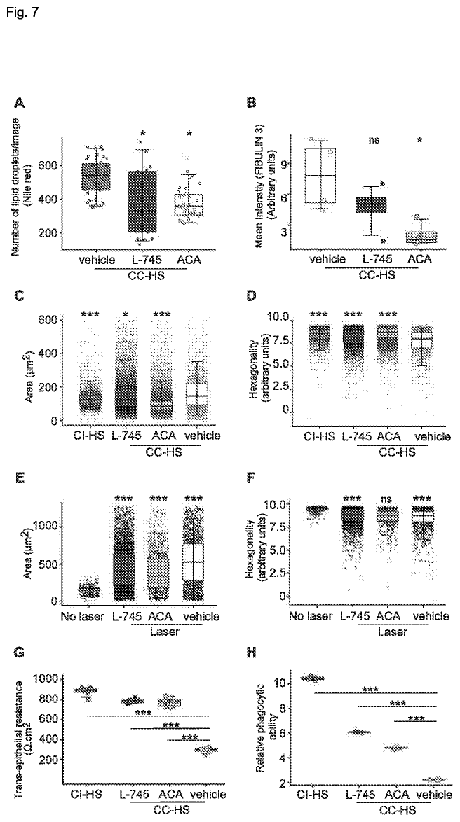

The key hallmark of AMD cellular phenotypes seen in cadaver eyes and observed in the in vitro model are the sub-RPE lipid and protein rich deposits, increased expression of drusen markers, and the loss of epithelial phenotype and RPE functionality. As compared to CC-HS treated samples, iRPE co-treated with CC-HS and L745,870 or ACA had 40-60% lower levels of −RPE lipid deposit, as measured by Nile red staining (FIG. 7A; CC-HS+ vehicle vs CC-HS+L, 745,870, p<0.01; CC-HS+ vehicle vs CC-HS+ACA, p<0.01) and four-fold lower expression of FIBULIN 3 (FIG. 7B; CC-HS+ vehicle vs CC-HS+ACA, p<0.01). Quantification of 3000-13,000 RPE cells revealed that, as compared to CC-HS+ vehicle treatment, cells co-treated with CC-HS and L745, 870 had an average area of 162.24 um2 (p<0.01) and hexagonality score of 8.25 (where 10 represents a perfect hexagon, p<10⁻¹⁵), and cells treated with CC-HS and ACA had an average area of 106.72 um2 (p<10⁻¹⁵) and hexagonality score of 8.52 (p<10⁻¹⁵) (FIGS. 7C, D). Similar results were obtained by RNAseq of iRPE treated with just CC-HS plus the vehicle or co-treated with CC-HS and the two drugs. Both drugs were able to suppress (4-10 fold) the expression of dedifferentiation markers in CC-HS treated iRPE cells (FIG. 13H).

To test the in vivo activity of these drugs, a rat model of RPE dedifferentiation was developed. A 0.5 mm area of rat RPE was damaged using a micropulse laser. RPE cells in the lasered area are ablated causing the cells surrounding the laser lesion to undergo dedifferentiation due to the loss of cell-cell contacts. These cells become enlarged and elongated with unorganized higher cytoplasmic expression of VIMENTIN, similar to CC-HS treated human cells. Effects of drugs on rat RPE dedifferentiation was tested by injecting either of the two drugs in the sub-retinal space of the rat eye at the time of laser injury. Quantification of 400-4000 RPE cells around the laser lesion revealed (ACA=397.53 um2, L-745,870=439.55 um2, laser=537.43 um2) 1 to 1.3 times smaller area in L745, 870 (p<10⁻¹⁵) and in ACA (p<10⁻¹⁵) injected cells as compared to lasered RPE (FIG. 7E). Drug treatment also improved hexagonality score from 6.91 in cells around laser lesion treatment to 7.42 to no drug injection treatment to X in CC-HS+L745, 870 treatment (p<10⁻¹⁴) (FIG. 7F). These in vivo experiments confirm the ability of these two drugs to restore epithelial phenotype of dedifferentiating RPE cells.

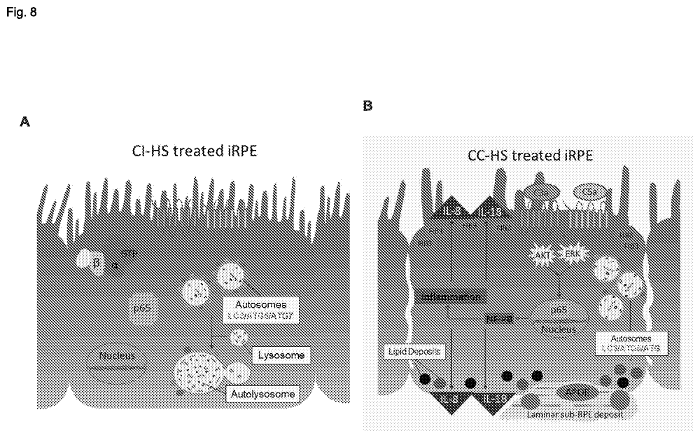

Lastly, it was checked if the drugs rescued mature RPE phenotype and RPE functionality. TER of samples co-treated with drugs and CC-HS was 2-3× higher (p<10⁻¹⁵) as compared to samples treated with vehicle+CC-HS was similar to TER values of CI-HS treated cells (FIG. 7G). Similarly, the addition of both drugs partially rescued (up to 2×; p<10⁻¹⁵) the ability of the RPE cells to phagocytose photoreceptor outer segments, as compared to cells treated with CC-HS and the vehicle (FIG. 7H). In conclusion, these results confirmed that the drugs, L, 745,870 and aminocaproic acid reversed the AMD cellular endophenotypes seen in CC-HS treated iRPE cells and restored RPE functionality and epithelial phenotype (FIG. 8). The in vitro and in vivo data provides sufficient evidence to support the potential use of these drugs to delay AMD disease initiation and slow its progression.

### Example 7—Mechanism of L-ORD and AMD and Use of Metformin as an Effective Therapy

In the present experiments, patient-specific iPSCs (induced pluripotent stem cells) were produced from members of a family affected with late onset retinal degeneration and their unaffected siblings and differentiated them into RPE to investigate the underlying disease mechanism. Under basal conditions patient and control subjects exhibited similar cobblestone-like morphology, and shared similar expression patterns of RPE-specific signature genes, and also stained positive for RPE-65, a mature RPE marker. The model was verified to accurately recapitulates the human disease phenotype in vitro by demonstrating that patient-RPE have altered cellular functions that mediate two key features of the disease: 1) elevated basal deposition of APOE, a demonstrated component of drusen and 2) excessive apical secretion of vascular endothelial growth factor (VEGF) a causative factor for the formation of CNV. The relationship between the mutation in CTRP5 was investiged and the disease-causing phenotypes observed. Contrary to what has been reported in the literature, CTRP5 is expressed and secreted in comparable levels between patients and unaffected siblings. Its receptor on the RPE was identified to be Adiponectin receptor 1 (AdipoR1), and not MFRP or Adiponectin receptor 2. AMPK is a sensor for the energy state of the cell, monitoring the ADP:ATP ratio, and is activated upon phosphorylation. Patient RPE were shown to be insensitive to changes in the energy status when placed under serum starvation. Additionally, this reduction in AMPK activity results in decreased utilization of photoreceptor outer segment (POS) which are rich in omega-3 lipids (DHA). This is manifested by reduced secretion of DHA-derived neurotrophic factors derived such as neuroprotectin D1 (NPD1). In this study, metformin, an anti-diabetic drug, is shown to rescue patient RPE cells by resensitizing AMPK to cellular stress (restoring energy homeostasis), restoring POS utilization, and repolarizing the secretions of both APOE and VEGF. To determine whether metformin may be an effective clinical therapy, a retrospective cohort study was performed of AMD patients concurrently treated with metformin for diabetes and found that metformin statistically improved clinical outcomes and delayed age of onset by two years. Taken together these results elucidate the disease mechanism that underlies L-ORD and AMD and demonstrates that ocular delivery of metformin is an effective therapy for patients with “dry AMD” who currently have no treatment options.

L-ORD is a rare inherited blinding disorder with presentation typically in the 5^{th}-6^{th} decades, and is characterized by yellowish punctate deposits in the fundus and delayed dark adaptation (night blindness). Unlike age-related macular degeneration (AMD), photoreceptor degeneration progresses from the periphery (rods) with subsequent loss of central cone vision gradually resulting in total vision loss. OCT revealed the presence of sub-retinal deposits as well as areas of separation between RPE and Bruch's membrane indicating sub RPE deposition suggesting that the observed loss in rod function may be secondary to dysfunction or death of the underlying RPE. Similar to AMD, the latter, advanced stages of the disease is frequently marked by progression to choroidoneovascularization (CNV) (Aye et al., 2010; Borooah et al., 2009; Cukras et al., 2016; Jacobson et al., 2014; Kuntz et al., 1996; Milam et al., 2000).

iPSCs were derived from skin fibroblasts taken from two patients with late-onset retinal degeneration (L-ORD) and two unaffected siblings. Fibroblast cultures were reprogrammed using Cytotune iPS 2.0 sendai reprogramming kit generating 2 clones per donor. All iPSC lines shared typical iPSC morphology, expressed pluripotency markers: OCT4, NANOG, SOX2, and SSEA4, and were karyotypically normal. An in vitro embryoid body (EB) assay demonstrated capability of differentiation into cell types from all three developmental germ layers (ectoderm, endoderm, mesoderm).

The 8 iPSC-RPE lines were considered as two groups: 4 healthy unaffected siblings, and 4 L-ORD patient iPSC-RPE. This grouping accounted for donor (genetic) and clonal variability as well as technical variability inherent with differentiation and cell culture conditions. Since modeling late onset neurodegenerative diseases with iPSCs has often proven difficult (Vera and Studer, 2015) due to the reprogramming process which resets the biological clock, using this approach to group and analyze the data provided a framework to make comparisons between multiple patients and controls to identify only relevant statistically significant differences that could be directly attributed to the disease phenotype (Schuster et al., 2015; Vitale et al., 2012).

In Vitro Model Replicates Clinical Disease Phenotype

The iPSCs were sequenced to verify that the patient lines retained the S163R point mutation (FIG. 14a). Using a previously published protocol the iPSCs were differentiated into RPE cells (Sharma et al., 2019a) and seeded onto transwells where they stably expressed markers of mature polarized RPE (TYR, PAX6, MITF, RLBP1, DCT, CLDN19, GPNMB, ALDH1A3, BEST1, TYRP1, and RPE65) (FIG. 14b). Transmission electron microscopy revealed a single monolayer of RPE cells with abundant apical processes (yellow arrow), apically localized melanosomes (magenta arrow), and a basally localized nuclei (white arrow) (FIG. 14c). Scanning electron images comparing L-ORD patient and unaffected siblings showed that iPSC-RPE grown on transwells took on classic hexagonal or cobblestone appearance but also revealed topographic differences in RPE cell size and shape suggesting that this heterogeneity was a feature of L-ORD patient RPE (FIG. 14(1). To determine whether differences in cell size distribution exist, quantitative image analysis was performed on iPSC-RPE stained with peripheral membrane proteins (Z0-1 or ADIPOR1) to outline cell borders. Cell area was found to be significantly larger and more variable in L-ORD patient RPE compared to unaffected siblings (FIG. 14e). TER measurements confirmed formation of normal RPE tight junctions (FIG. 14f) and did not reveal any differences between lord patient specific iPSC-RPE and unaffected healthy siblings. Morphological and phenotypic changes are often associated with RPE dedifferentiation—a process by which epithelial cells lose their cell polarity and cell adhesion. However, under normal culture (nonstressed) conditions, the expression patterns of genes related to dedifferentiation in L-ORD patient RPE is similar to that observed in unaffected siblings (FIG. 14g). Under the same conditions, cells were lysed and the underlying basal apolipoprotein E (APOE) deposits, which have been shown to associate with high density lipoproteins (HDLs) and involved with lipid trafficking (Ishida et al., 2004), were stained and analyzed indicating slight differences in the overall quantity and distribution in L-ORD patients versus unaffected siblings (FIG. 14h). Lastly, L-ORD patient RPE exhibited loss of polarized secretion of vascular endothelial growth factor (VEGF) (FIG. 14i). In particular, the basal VEGF secretion was reduced by approximately 50% (p=0.046) in L-ORD patient RPE (n=7) compared to unaffected siblings (n=3). Taken together, these results suggest that the iPSC-RPE model preserved phenotypic changes associated with the disease-causing mutation in CTRP5.

CTRP5 is an Autocrine Regulator of RPE Metabolism

The CTRP5 protein, which is known to have a S163R point mutation in L-ORD, is produced by a bicistronic gene that also encodes membrane frizzled related protein (MFRP). qRT-PCR was performed to determine if the point mutation altered the mRNA expression of CTRP5 or MFRP in L-ORD patients. Delta Ct values comparing CTRP5 and MFRP were similar across different donors and clones indicating no patient-specific differences in expression (FIG. 15a). Interestingly, western blots of CTRP5 indicated reduced protein expression in cell lysates of L-ORD patient RPE (FIG. 15b). FIG. 15c the CTRP5 protein levels in L-ORD patient iPSC-RPE were quantified by densitometry and normalized to β-actin and indicated a 7-fold decrease in expression. CTRP5 Elisa and WBs indicate that CTRP5 is apically secreted (FIG. 15d). The measured CTRP5 from the basal chambers was below the detection limit (data not shown). To determine the mechanism through which a mutation in CTRP5 brings about the disease phenotype in L-ORD, specific ligand-receptor interactionswere identified using super-resolution microscopy to screen putative candidate receptors based on the homology of CTRP5 to adiponectin (Yamauchi et al., 2014; Yamauchi and Kadowaki, 2013) and its reported interaction with membrane frizzled related protein (MFRP) (Mandal et al., 2006; Shu et al., 2006) Immunofluorescence confocal microscopy revealed co-labeling of CTRP5 (red-ligand) with adiponectin receptor 1 (ADIPOR1, green-receptor) (FIG. 15e). Native immunogold labeling of cultured human iPSC-RPE confirmed CTRP5 (12 nm) and ADIPOR1 (6 nm) interaction (FIG. 150. FIG. 15g displays a model of the integral membrane protein, ADIPOR1 (blue), and its interaction with CTRP5 (teal) taking into account 3D-structural constraints to determine probabilistic interaction. As shown in FIG. 15g, like adiponectin, CTRP5 forms trimers as its fundamental structural unit but also tends to form higher order structures resembling bouquet-like arrangements (Tu and Palczewski, 2012). This model was used to simulate the S163R mutation in L-ORD. The acquisition of a positively charged arginine alters CTRP5's electrostatic interaction with ADIPOR1 by repelling a similarly charged arginine on ADIPOR1's surface—weakening its binding affinity for the receptor (FIG. 15h).

CTRP5 Fine Tunes AMPK Sensitivity to Cell Energy Status

Adiponectin and its receptors are known to regulate lipid metabolism in an AMP-activated protein kinase (AMPK) dependent mechanism. Thus, the following experiments were designed to determine whether the mutation in CTRP5 altered AMPK activation and signaling pathways regulating energy homeostasis.

At baseline in serum containing media, the levels of phospho-AMPK (p-AMPK), a measure of AMPK activity, was 20% higher in L-ORD patient iPSC-RPE compared to unaffected siblings (p<0.05) (FIG. 16a).

Thus, in FIG. 16b iPSC-RPE were incubated with recombinant CTRP5 globular form (0.2 μg/mL gCTRP5) for 30 min in the presence (+) and absence (−) of serum to evaluate its role in AMPK signaling. The addition of gCTRP5 to iPSC-RPE from siblings and patients in serum containing media did not alter AMPK activity. However, in serum deprived media the addition of gCTRP5 led to a 20% decrease in p-AMPK levels in unaffected siblings but not in L-ORD patients (p<0.05). These data provide evidence that CTRP5 acts as a metabolic knob for fine tuning AMPK levels to meet the energy demands of the cell.

To better elucidate the ADIPOR1-CTRP5 (receptor-ligand) interaction, rmed a ligand dose response curve was performed (in serum free media) by exogenously adding increasing concentrations of recombinant full length CTRP5 (FIG. 16c). In unaffected siblings, dose-dependent increases in CTRP5 resulted in a significant decrease in p-AMPK levels (50% reduction at 25 μg/mL, p<0.05), whereas in L-ORD patients, the addition of CTRP5 had no effect on AMPK activity. These findings suggest that the mutant CTRP5 associates with native CTRP5 and forms complexes (higher order structures) that perturb its normal biological activity.

AMPK is a sensitive indicator of the cell energy status and is canonically activated by the levels of AMP or ATP (Hardie and Lin, 2017). Therefore, the AMPK activity (in serum free media) of sibling and patient iPSC-RPE under conditions (increased AMP:ATP ratio) known to stimulate AMPK phosphorylation was characterized (FIG. 16d). iPSC-RPE were incubated in serum deprived media for 5 hours (Park et al., 2009) followed by 30 mins exposure to AICAR (an AMP analogue) or BAM15 (a mitochondrial uncoupler to reduce ATP production). As expected, in iPSC-RPE derived from unaffected siblings increases in the AMP:ATP ratio activated AMPK. Interestingly, under the same experimental conditions L-ORD patient iPSC-RPE failed to sense changes in the AMP or ATP levels. These findings suggest that a failure to sense AMP or ATP is the key mechanism underlying incongrous AMPK activation in L-ORD patients and may be novel target for therapeutic intervention.

Adiponectin is also known to stimulate ceramidase activity to promote cell survival (Kadowaki and Yamauchi, 2011). As reported by Dr. Lakkaraju's lab, excess ceramide at the apical surface of the RPE may be a pathological feature that leads to intracellular complement (C3a) generation and mTOR reprogramming of RPE metabolism (Kaur et al., 2018). Consistent with increased AMPK activation in patient cells, immunostaining for ceramide in L-ORD patient iPSC-RPE (en face images) did not reveal excessive ceramide compared to unaffected siblings implying that the AMPK defect described above may be the primary mediator of disease pathogenesis in L-ORD.

AMPK is a central regulator of a multitude of metabolic pathways that may contribute to the disease phenotype observed in L-ORD. A gene expression profile of AMPK related genes revealed that PNPLA2 (PEDF-R) was highly expressed in iPSC-RPE of L-ORD patients. Interestingly, elevated levels of p-AMPK have been reported to increase expression on PEDF-R in skeletal muscle cells (Wu et al., 2017) Immunohistochemistry confirmed increased protein expression of PEDF-R (red) in L-ORD patient iPSC-RPE compared to unaffected siblings (FIG. 160. Collectively, these results suggest that AMPK-perturbed levels of PEDF-R may contribute to (can trigger) age-related pathological changes in L-ORD patient (human) RPE.

Elevated AMPK in L-ORD Disrupts PEDF-R Mediated Retinal Neuroprotection

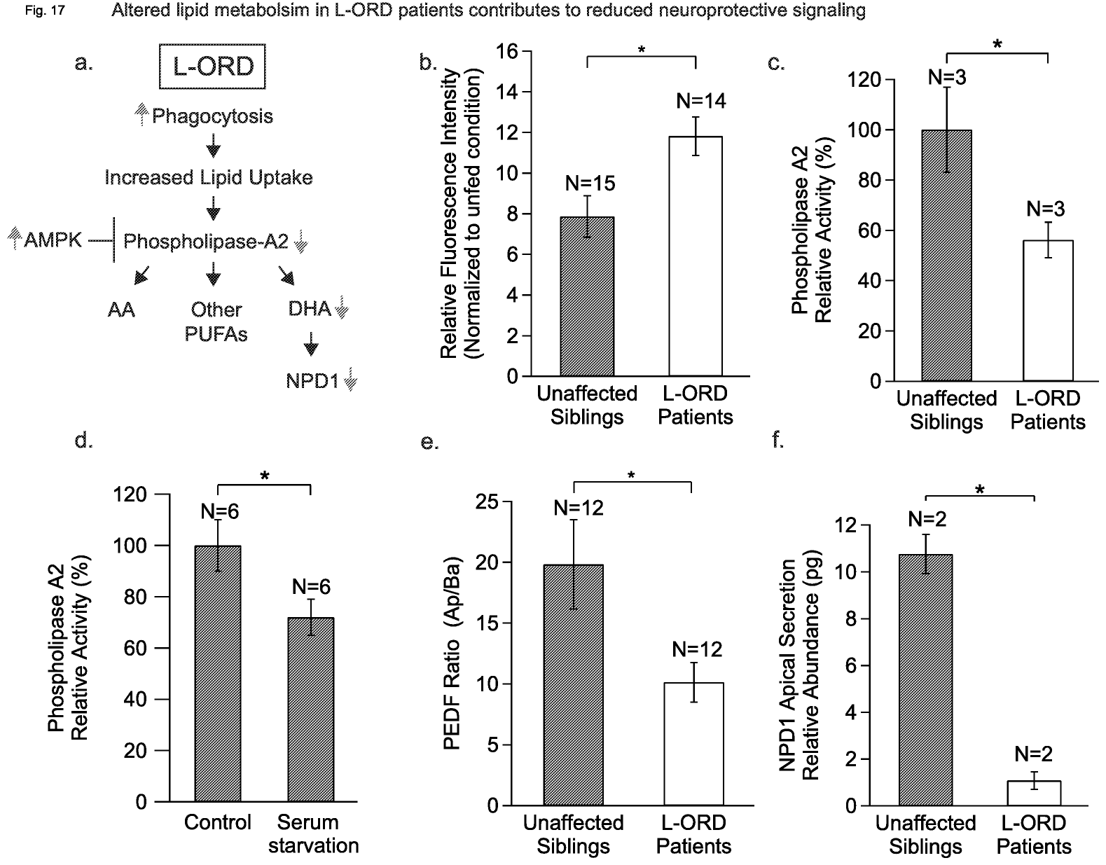

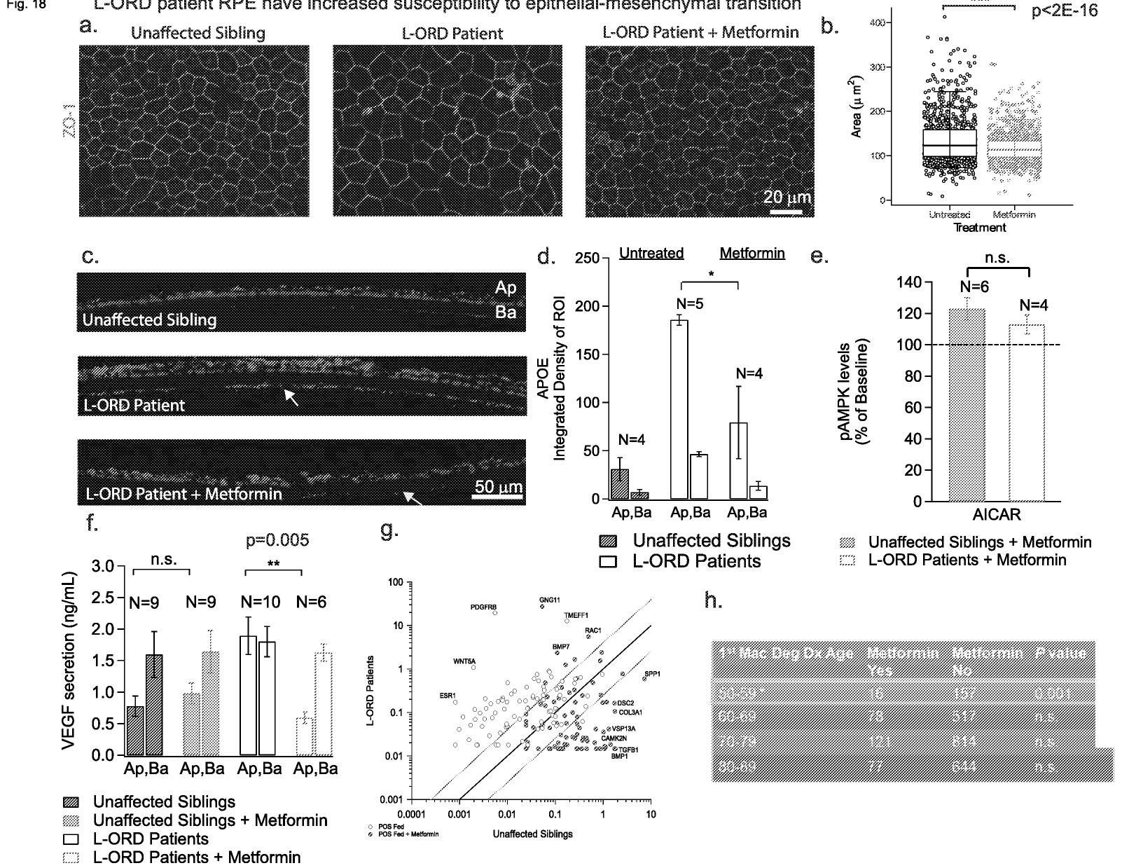

To investigate how the RPE pathology occurs, a comparative study of L-ORD patients and unaffected siblings was performed in the context of PEDF-R mediated neurotrophic signaling and its role as an angiogenesis inhibitor. FIG. 17a presents a model through which the mutation in CTRP5 disrupts normal homeostasis in the aging RPE resulting in an imbalance in lipid uptake and utilization. L-ORD patient iPSC-RPE exhibit elevated phagocytic capacity, a phenomenon that has been reported in RPE cells to compensate for oxidative-stress induced apoptosis (Mukherjee et al., 2007). The increased lipid uptake necessitates increased phospholipase A2 activity to cleave the phospholipids and produce free fatty acids that serve as the precursor molecules for biologically active compounds such as DHA, eicosanoids, and neuroprotection D1 (NPD1). In L-ORD patient iPSC-RPE however, the elevated levels of AMPK at baseline inhibit phospholipase-A2 enzyme activity stunting the production of these neuroprotective factors. In FIG. 17b, FACS analysis of iPSC-RPE fed phRodo labeled outersegments reveals that L-ORD patient iPSC-RPE phagocytose 1.5 times more POS compared to unaffected siblings. Despite the increased lipid intake, the phospholipase a2 activity at baseline in L-ORD patient iPSC-RPE is reduced by ˜40%, likely due to increased AMPK activity (p<0.05, FIG. 17c). In iPSC-RPE, the enzymatic activity of phospholipase A2 is dependent on pAMPK levels (FIG. 17d). Elevated pAMPK levels brought on by serum starvation in WT iPSC-RPE reduces phospholipase A2 activity by ˜30% (p<0.05) mimicking the condition observed in L-ORD patients. In RPE, PEDF is secreted in a polarized fashion, predominantly apical (Maminishkis et al., 2006; Sonoda et al., 2009). However the PEDF Ratio (Ap/B a) of PEDF secretion is significantly lower in L-ORD patient iPSC-RPE (FIG. 17e). Together the lower PEDF-R enzymatic activity coupled with the reduced amounts of apical PEDF result in significantly lower mitochondrial function (basal respiration, proton leak, atp production) and decreased neuroprotection D1 (NPD1) apical secretion (p<0.05). Collectively these results demonstrate how altered lipid metabolism in L-ORD patients contributes to reduced PEDF-mediated neuroprotection of photoreceptors.

Metformin Resensitizes AMPK and Pathological Phenotype

Accumulating evidence implicates disrupted lipid metabolism as a common pathogenic mechanism in a host of diseases including AMD (Ban et al., 2018; Xu et al., 2018). These metabolic defects are linked to long term health of RPE cells. To determine the consequence of PEDF-R deficiency in RPE cells, L-ORD iPSC-RPE and unaffected siblings were subjected to 7-day photoreceptor outersegment (POS) feeding to exacerbate the high fat-induced epithelial impairment. Concomitantly, it was investigated whether the lipid-lowering effects of metformin, an anti-diabetic drug (Schneider et al., 1990), could reverse RPE dedifferentiation and loss of polarity. Cell size, a key morphological indicator of RPE polarity, was evaluated by staining for ZO-1 (green) to identify cell borders (FIG. 18a) and employing automated image analysis algorithms in REShAPE (a cloud-computing based cell morphometry analyzer) to perform morphometric analysis of RPE cells.

Patient iPSC-RPE were treated daily beginning with the first day of POS feeding with 3 mM metformin (Fan et al., 2015; Kim et al., 2011). Patient iPSC-RPE fed POS exhibited increased cell size and variability, even compared to unfed cells. Similar morphometrics have been reported in human AMD eyes that also exhibited strong spatial irregularities (Rashid et al., 2016). Notably, metformin treatment largely prevented the POS stress-induced enlargement of patient iPSC-RPE (p<2e-6) (FIG. 18b). FIG. 18c shows that in similarly POS-fed iPSC-RPE the distribution of apolipoprotein E (APOE), a major constituent of very low-density lipoproteins (VLDL) and high-density lipoproteins (HDL) is affected.

In unaffected siblings, APOE is secreted from the RPE's apical and basal surfaces but was found to be primarily apical (FIG. 18c, top), where it may play a role in lipid trafficking (Ishida et al., 2004). In contrast, L-ORD patient iPSC-RPE exhibited increased levels of APOE. White arrow indicates basal increase in APOE deposits reminiscent of APOE-rich drusen deposits in AMD (Johnson et al., 2011). Patient RPE treated with metformin ameliorate the accumulation of APOE-containing basal deposits (yellow arrow). Image J was used to draw a rectangular region of interest around the apical and basal APOE-positive staining to quantify the localized mean integrated intensity and the data is summarized in FIG. 18d.

In FIG. 18e, L-ORD patient iPSC-RPE pretreated with metformin (1½ weeks) resulted in restored sensitivity to AICAR (an AMP analogue). This result suggests that metformin restored normal energy homeostasis and may have clinical value.

POS feeding for 7-days also altered the expression profiles of 85 EMT-related genes in L-ORD patient iPSC-RPE (FIG. 18g). Compared to unaffected siblings, L-ORD patient iPSC-RPE upregulated 54 genes >4-fold (white) associated with epithelial-mesenchymal transition (e.g. ESR1, WNT5a, PDGFRB, GNG11, TMEFF1, BMP7, and RAC1). In contrast, L-ORD patient iPSC-RPE treated with metformin (magenta) downregulated 42 EMT <4-fold genes (e.g. SPF, DSC2, COL3A1, VSP13A, CAMK2N, TGFB1, BMP1). Taken together, these data indicate that L-ORD patient iPSC-RPE are 1) susceptible to lipid-stress induced epithelial mesenchymal transition and 2) metformin can resensitize AMPK alleviating pathological changes in cell size, APOE deposition, VEGF secretion, and gene expression.

Additionally hypoxic microenvironments that accompany aging, have been shown to similarly alter lipid metabolism (Kurihara et al., 2016). Hypoxic stress (3% 02, 6h) was employed to determine how this perturbation regulates VEGF secretion in L-ORD patient iPSC-RPE. Similar to the normoxic condition L-ORD patients exhibited mispolarized VEGF secretion with elevated levels of apical secretion, an indicator of dedifferentiation (EMT). Treatment with metformin for 24h is insufficient to rescue this phenotype (data not shown) suggesting that metformin exerts its effects primarily by altering gene expression (FIG. 18e). In support of this hypothesis, 1½ week pretreatment with metformin alleviated the hypoxia-induced apical VEGF secretion (p=0.005) and restored VEGF polarity (FIG. 18f).

To determine whether there is clinical evidence that metformin could be beneficial in the treatment of AMD, a retrospective cohort study of patients presenting to Kaiser Permanante Medical with AMD was performed to test whether concurrent use of metformin by individuals affected diagnosis. For individuals belonging to the age group between 50-59 years of age, generally considered early-onset (Schick et al., 2015), this study revealed that metformin significantly delayed the age of onset by 2 full years (p=0.001).

Collectively these results suggest that metformin or AMPK sensitizing drugs can restore the RPE phenotype and be a potential treatment for dry AMD.

### Example 8—Analysis of iPSC-RPE from Patients with Late-Onset Retinal Degeneration Identifies the Role of AMPK in Regulating Healthy RPE Phenotype and LED to a Re-Purposing of Metformin, a Known Type 2 Diabetes Drug for a Potential Treatment of AMD and Other Retinal Degenerative Diseases

Late-onset retinal degeneration (L-ORD) is a rare, inherited, monogenic retinal dystrophy that shares many of the same clinical phenotypes of more common retinal degenerations such as age related macular degeneration (AMD) (drusen-like deposits, choroidal neovascularization that can develop late in the disorder).

Underlying L-ORD is a mutation in CTRP5 which is similar in structure to adiponectin a known adipokine that is an important regulator of glucose and lipid metabolism-altered metabolism has been associated with many forms of retinal degeneration.

Late Onset Retinal Degeneration, or L-ORD, presents with pathology similar to AMD, usually after the age of 40. L-ORD patients have delayed dark adaptation, which indicates a problem in the photoreceptors and the visual cycle. Furthermore, they had drusen like deposits, which showed up as hyperfluorescent deposits on the FAF. Finally they had disrupted inner and outer photoreceptor segments seen in OCT.

L-ORD is caused by a single missense mutation in CTRP5, an adiponectin paralog that is highly expressed in the RPE. CTRP5's globular domain is 40% homologous to adiponectin, indicating a possible role in cell metabolism.

A critical readout of cellular metabolism is AMPK a critical energy sensor that monitors the ratio of ATP/AMP and is phosphorylated (activated) during nutrient deprivation. The inventors hypothesized that because patients have hyperactive pAMPK levels (data not shown) that the cells will become insensitive to changes in ATP and AMP. Under conditions of serum starvation (3h), pAMPK levels are elevated compared to baseline in both L-ORD patients and unaffected siblings.

The addition of AICAR (30 min), an AMP analogue stimulates further phosphorylation of AMPK in unaffected siblings but not in L-ORD patients. The addition of BAM15 (30 min), a mitochondrial uncoupler that inhibits ATP production, also further stimulates phosphorylation of AMPK in unaffected siblings but not in L-ORD patients. In effect, L-ORD patients are insensitive to changes in ATP or AMP under serum starvation conditions. Metformin treatment consisted of 3 mM metformin added to the apical and basal media for 1½- 2 weeks. L-ORD patients treated with metformin regained sensitivity to AICAR following serum starvation.

Alterations in the AMPK signaling pathway in L-ORD patients was further assessed through gene expression revealing a compensatory down regulation in many of the genes involved. Treatment with metformin (3 mM) for 1½ to 2 weeks results in further decrease in some genes but an upregulation in others. In particular there is a significant increase in PNPLA2 (PEDF-R). In the RPE, the PEDF-R plays an important role in fatty acid metabolism.

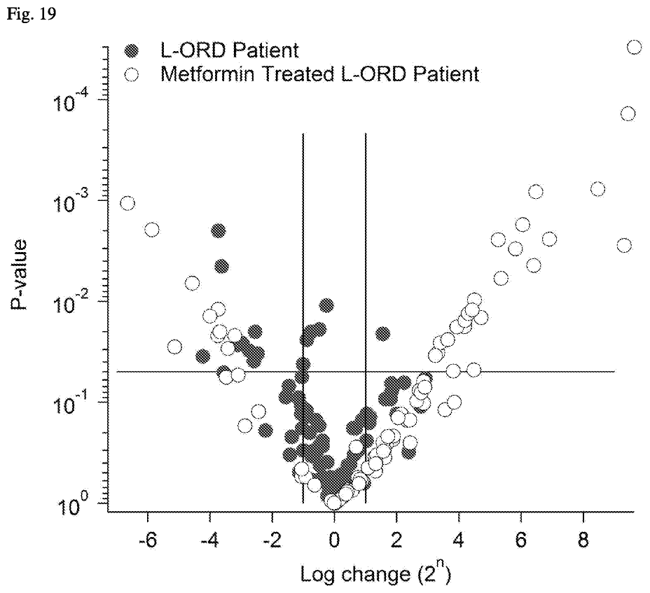

In FIG. 19, it is shown that the gene expression profile of L-ORD patients suggest a compensatory attempt to limit activation of pAMPK at baseline.

Unaffected Siblings (N=8), from N=2 donors

(2 from 24G, 2 from Z8, 2 from Y9, 2 from 9i)

LORD-Patients (N=7), from N=2 donors

(3 from Donor E1, 4 from donor K8)

Metformin Patient (N=4), from N=2 donors

Polarized secretion of cytokines by the RPE is a hallmark of their mature and differentiated state. Under conditions that promote epithelial to mesenchymal transition (EMT), RPE lose their morphology and their secretion becomes mispolarized. Dedifferentiation of the RPE is a frequently proposed mechanism in retinal degenerations such as AMD. Typically VEGF is primarily secreted basally by the RPE. In L-ORD patients, the polarity of VEGF secretion is lost as assessed by ELISA. In contrast treatment with metformin (3 mM) for 1½ to 2 weeks results in a rescue of polarized VEGF secretion suggesting that metformin may mediate EMT inhibition. This is shown in FIG. 19

Untreated:

Unaffected Siblings

Apical N=7

Basal N=3

L-ORD Patients

Apical N=7

Basal N=3

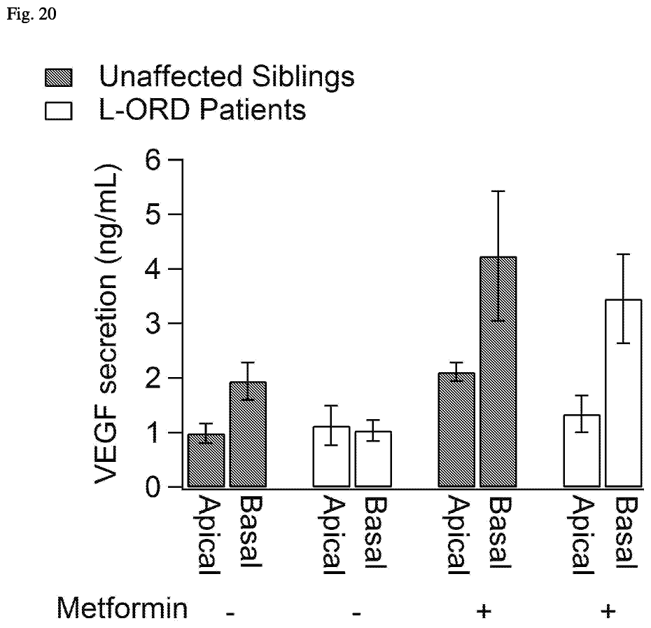

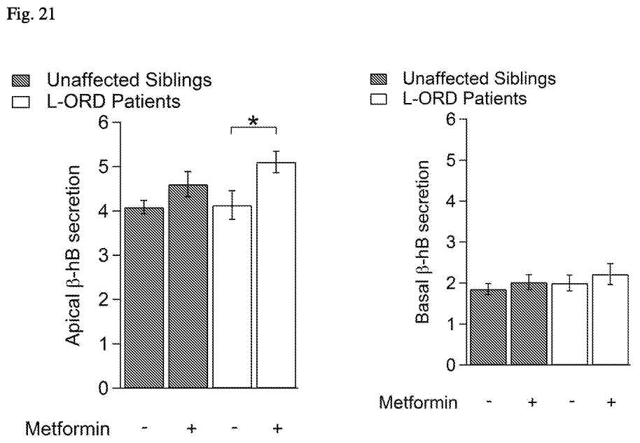

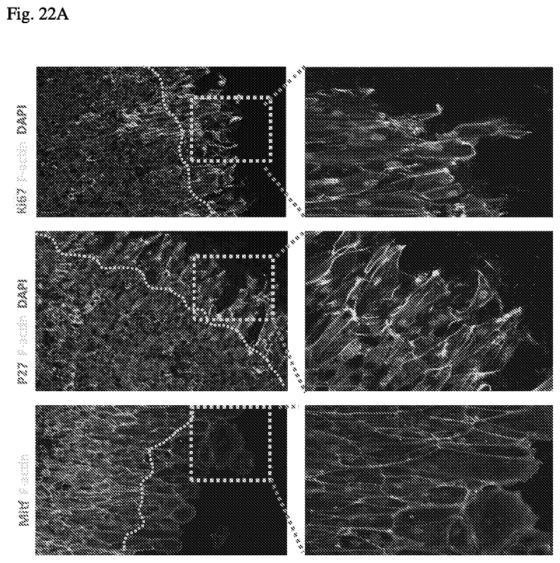

FIG. 20 demonstrates that B-hB is generated by the RPE which utilizes the fatty acids derived from phagocytosed photoreceptor outer segments (of which palmitate is a major component) and generates self sustaining metabolites through a process called fatty acid oxidation thus sparing glucose for the retina. The inventors have hypothesized that increasing fatty acid oxidation and ketone body formation (B-hB) may lead to decreased lipid accumulation in the sub retinal space. Metformin treatment resulted in a significant 25% increase in apical B-hB secretion by L-ORD patient RPE.

Metformin treatment consisted of 3 mM metformin added to the apical and basal media for 1½- 2 weeks.

Each bar plot represent N=12 biological replicates compiled from 2 different donors (either unaffected siblings or patients). * indicates pvalue <0.05

### Example 9—Metformin Delays Median-Age of Onset for Retinal Degenerative Diseases

The drug Metformin (Brand names: Fortamet, Gluophage, Glumetza, Riomet) is widely used to treat type 2 diabetes (T2D). The safety profile of Metformin has been widely established based on years of use in both US and European markets. Metformin was first marketed in 1958 in the U.K. by Rona a subsidiary of Aron laboratories for its potent effect to lower blood glucose in diabetic patients and was later found to activate AMP-activated protein kinase AMPK enzyme to normalize cellular metabolism and blood glucose levels.

To determine whether there is clinical evidence that metformin could be beneficial in the treatment of AMD a retrospective cohort study was performed of patients presenting to Kaiser Permanante Medical with AMD to test whether concurrent use of metformin by individuals affected diagnosis. For individuals belonging to the age group between 50-59 years of age, generally considered early-onset (Schick et al., 2015), this study revealed that metformin significantly delayed the age of onset by 2 full years (p=0.001). The Results of this study are shown in FIGS. 14-18

FIGS. 14a-14i depict various testing data which demonstrates that patient-specific iPSC-RPE retained a disease-causing mutation. a) Sanger sequence analysis confirms the presence of the S163R mutation in iPSCs derived from patients with L-ORD. The sequences are shown on top and the base affected by the mutation is indicated on the sequence chromatogram by the black arrow. The heterozygous point mutation (AGC->AGC, AGG) appears as a peak within a peak. Primers for DNA sanger sequencing are described in Methods. b) boxplot diagrams of deltaCt values of the indicated RPE signature genes. Each box represents the distribution of the deltaCt measured from n=3 iPSC-RPE from at least 2 different unaffected siblings or L-ORD patient donors. Bottoms and tops of the boxes define the 10^{th} and 90^{th} percentile. The band inside the box defines the median. c) Transmission electron microscopy images of iPSC-RPE monolayers fed photoreceptor outersegments for 7 consecutive days. TEM of iPSC-RPE derived from an unaffected sibling (above) and patient (below) showing normal RPE morphology and highly polarized structure including abundant apical processes (yellow arrow), melanosomes (magenta arrow), and basally located nuclei (white arrow). Scale bar: 2 μm. d) SEM images of iPSC-RPE derived from unaffected siblings and L-ORD patients showing preserved hexagonal morphology and abundant apical processes. e) Box plot of cell area of iPSC-RPE derived from unaffected siblings and L-ORD patients. iPSC-RPE monolayers were immunostained with a membrane marker (ADIPOR1) to outline their hexagonal shape for multiparametric analysis of cell morphology. L-ORD patient iPSC-RPE tended to be larger in size on average (107.7+/−68.5 μm²) and more variable compared to unaffected siblings (79.8+/−57.5 μm²) (p=0.000026). Similar spatial irregularities have been reported in the eyes of human AMD donors (Rashid, A. et al. RPE Cell and Sheet Properties in Normal and Diseased Eyes. Adv Exp Med Biol 854, 757-763, (2016)). f) Establishment of functional tight junctions between iPSC-RPE cells was measured by transepithelial resistance measurements using an EVOM epithelial voltohmmeter (World Precisions Instruments). The disease associated missense mutation does not alter the transepithelial resistance of the RPE monolayer. g) Scatter plot of genes enriched in RPE cells that undergo dedifferentiation (epithelial mesenchymal transition) reveal that under normal conditions L-ORD patient cells do not show an abnormal phenotype indicative of diseased or stressed RPE. The expression of dedifferentiation (EMT)-related genes in unfed (shown in gray) patient iPSC-RPEs resemble the expression patterns of unfed unaffected siblings. h) iPSC-RPE derived from unaffected siblings and L-ORD patients subjected to normal culture conditions show similar levels of APOE basal deposits. Scale bar: 50 μm. i) The release of VEGF by iPSC-RPE into the supernatant under normoxic conditions was measured by ELISA. The highly polarized structure of RPE is responsible for vectorial transport and secretion of proteins including VEGF. Naturally, iPSC-RPE derived from unaffected siblings (shown in gray) secreted VEGF in a polarized manner, predominantly basal. L-ORD Patient derived iPSC-RPE exhibit a loss of polarity with approximately a ˜53.3% reduction in basal VEGF secretion (P=0.046).

FIGS. 15a-15h depict various testing data which demonstrates expression and localization of CTRP5 in L-ORD patient-derived RPE. a) In L-ORD the S163R mutation occurs in a bicistronic transcript that codes for CTRP5 (a secretory protein) and membrane frizzled related protein (MFRP). The mutation does not alter the mRNA expression of either transcript. b) Representative western blot of cell lysate from iPSC-RPE of unaffected siblings and L-ORD patients. Since CTRP5 is a secreted protein, the strong 25 kDa band (CTRP5) in the unaffected siblings may indicate CTRP5 is retained to a greater degree in the whole cell extract. c) Quantification of western blot (cell lysate) normalized to β-actin (p<0.05). d) In iPSC-RPE from unaffected siblings and L-ORD patients CTRP5 was selectively secreted to the apical side as measured by ELISA following 48 hours. No measureable difference was observed between the amounts secreted by unaffected siblings and patients. Negligible amounts of CTRP5 were detected in the basal media (data not shown). e) Airyscan confocal microscopy images of immunofluorescent stainings of iPSC-RPE from unaffected siblings and L-ORD patients. The membrane receptor ADIPOR1 (shown in green) co-localizes with CTRP5 (shown in red), HOESCHT (nuclear stain shown in blue). f) TEM image of native immunolabeled ADIPOR1 (6 nm immunogold) and CTRP5 (12 nm immunogold) provide evidence of receptor-ligand interaction (indicated by black arrow). g) 3-D model of protein-protein interaction between ADIPOR1 (shown in blue) and CTRP5 (shown in green) using published crystallographic structures. h) The Serine (polar) to Argenine (+) mutation alters the charge of the residue making it positive. This positive charge is predicted to repel a neighboring argenine residue and results in a conformational change that reduces the binding affinity of the mutant CTRP5 to ADIPOR1.

FIGS. 16a-16f depict various testing data which demonstrates reduced antagonism of CTRP5 on ADIPOR1 results in altered AMPK signaling in L-ORD. a) Phospho-AMPK levels determined by ELISA indicate approximately a 20.6% increase in baseline activity in L-ORD patient iPSC-RPE (N=15; (120.6%±0.075) cultured in 5% serum containing media compared to unaffected siblings (N=21; 100%±0.04). b) Influence of recombinant globular CTRP5 on phospho-AMPK levels in the presence and absence of serum containing adiponectin. Data are normalized to the untreated condition (0 ug/mL gCTRP5). In unaffected siblings, the addition of 0.2 μg/mL of recombinant globular CTRP5 in the absence of the natural ligand, adiponectin (under 0% serum conditions) reveals a 20% decrease in pAMPK levels (N=9; 0.81±0.04). This significant decrease is masked by the presence of 5% serum under baseline conditions (N=6; 0.99±0.01). In L-ORD patient iPSC-RPE, the addition of 0.2 μg/mL recombinant globular CTRP5 has no measurable effect on the p-AMPK levels (N=6; 1.12±0.09) even in the absence of serum (N=6; 0.98±01). c) Dose-response effects of recombinant full length CTRP5 on the p-AMPK levels of iPSC-RPE derived. In unaffected siblings (5h 0% serum), the phosphorylation levels of AMPK are reduced after treatment (30 min) with increasing concentrations of recombinant full length CTRP5. 25 ug/mL CTRP5 results in a ˜50% reduction in p-AMPK levels (N=6, 47.89%±0.13). Patient RPE subjected to similar concentrations of full length CTRP5 elicited no measurable change in p-AMPK levels. d) Conditions that elevate the AMP:ATP ratio in the absence of serum result in altered p-AMPK levels in patient derived iPSC-RPE compared to unaffected siblings. All data are normalized to the 0% serum containing condition. 30 min treatment with 2 mM AICAR, an AMP analog, or 500 nM BAM15, a mitochondrial uncoupler that reduces ATP production, results in further elevation in AMPK levels in unaffected siblings. In contrast the p-AMPK levels of patient RPE are insensitive to changes in AMP or ATP levels. However two-week treatment with 3 mM metformin restores the sensitivity of the L-ORD patients to changes in the AMP:ATP ratio. e) Elevated AMPK in L-ORD patient derived iPSC-RPE results in significantly upregulated mRNA expression of PEDF-R (˜8-fold). f) Immunohistochemistry confirmed elevated PEDF-R protein expression localized to the apical membrane in L-ORD patient iPSC-RPE.

FIGS. 17a-17f depict various testing data which demonstrates altered lipid metabolism in L-ORD patients contributes to reduced neuroprotective signaling. a) Presumptive model depicting the phagocytic uptake of lipid-rich outer segments and their digestion by phospholipase into free fatty acids that the RPE utilizes for ketogenesis and the synthesis of neuroprotective lipid mediators such as NPD1. In human cancer cell lines, elevated p-AMPK levels have been shown to suppress phospholipase D activity (Mukhopadhyay, S. et al. Reciprocal regulation of AMP-activated protein kinase and phospholipase D. J Biol Chem 290, 6986-6993, doi:10.1074/jbc.M114.622571 (2015)) and is the proposed mechanism through which increased lipid uptake in L-ORD patients results in decreased utilization and synthesis of DHA-derived Neuroprotectin D1 and an accumulation of undigested lipids. b) The uptake of ph-Rhodo labeled outersegments were quantified by FACS to compare the phagocytic rate of iPSC-RPE derived from unaffected siblings and L-ORD patients. The phagocytic uptake of L-ORD patient iPSC-RPE (N=14; 11.81±3.55) was 33% higher than unaffected siblings (N=15; 7.86±3.94). This phenomenon of increased lipid uptake has been reported in RPE as a protective response to oxidative stress. (Mukherjee, P. K. et al. Photoreceptor outer segment phagocytosis attenuates oxidative stress-induced apoptosis with concomitant neuroprotectin D1 synthesis. Proc Natl Acad Sci USA 104, 13158-13163, (2007)). d) Despite a significant increase in overall PEDF-R expression, L-ORD patient phospholipase A2 activity was measured by ELISA to be 40% lower than unaffected siblings. e) Phospholipase A2 activity is shown to be significantly reduced (26%) in normal iPSC-RPE (n=6) subjected to elevated levels of pAMPK (n=6, induced by serum starvation) (p<0.05). f) The polarized secretion of PEDF was determined by ELISA. L-ORD patients (N=12) exhibited reduced apical (patient: 939.6 ng/mL/sibling: 1277.22 ng/mL) and increased basal (patient: 92.16 ng/mL/sibling: 75.96 ng/mL) secretion of PEDF, resulting in a significantly reduced PEDF ratio (Ap/Ba) (10.13±1.63) compared to unaffected siblings (N=12, 19.82±3.67) (p=0.0014). Data are mean±SE and represent the average of 3 independent experiments. * indicates is p<0.05. f) Apical secreted DHA-derived neuroprotection D1 was measured by tandem mass spectrometry lipidomic analysis. Unaffected siblings (Z8,9i) secreted approximately ˜10 times more NPD1 than L-ORD patients (K8,E1) (p=0.0089).

FIGS. 18a-18h depict various testing data which demonstrates L-ORD patient RPE have increased susceptibility to epithelial-mesenchymal transition. Representative images showing immunofluorescent staining of the membrane marker ZO-1 (shown in green) of iPSC-RPE following 7 consecutive days of feeding photoreceptor outer segments. All images were obtained using a 63× objective. Scale bar=20 um. b) Images obtained under conditions described in (a) were subjected to shapemetric analysis to construct box plots of the distribution of cell area (Low whisker: 5% of data, Low hinge: 25% of data, Midline: Median, High hinge: 75% of data, High whisker: 95% of data). L-ORD Patient iPSC-RPE (N=6 images, 135.37±1.76 um) possess increased cell size and variability compared to unaffected siblings (N=5, 95.77±1.68 um) (p<2E-16). In unaffected siblings, metformin treatment initiated during photoreceptor feeding had minimal effect on cell area (N=7, 93.14±1.56 um) compared with untreated unaffected siblings (p=0.52). However, 3 mM metformin treatment resulted in a significant decrease in patient cell area (N=7, 117.92±0.96 um) compared to untreated patients (p<2E-16). Dunnett's multiple comparison test was performed to compare either to untreated unaffected siblings or L-ORD patients. c) Immunofluorescent microscopy images of APOE stained cryosections of iPSC-RPE monolayers following 7-days POS feeding. L-ORD patient iPSC-RPE exhibited altered relative proportions of apical and basal APOE deposition (white arrow). L-ORD patients treated with metformin during POS feeding resulted in a redistribution of the relative proportions of apical and basal APOE deposition (yellow arrow) resembling unaffected siblings. d) Image quantification of the integrated density of APOE signals of images similar to those shown in c). Integrated density of APOE signal is significantly higher in untreated L-ORD patients (N=5; Apical: 185.69±5.42; Basal: 46.38±2.51) compared to unaffected siblings (N=4; Apical 30.89±12.05; Basal: 8.45±3.09) (Apical: p=7.76E-6; Basal: p=2.71E-5). No significant difference between metformin treated L-ORD patients (N=4; Apical 79.30±37.51; Basal: 13.58±4.58) compared to metformin treated unaffected siblings (N=8; Apical 119.98±20.36; Basal: 23.55±6.17) (Apical: p=0.32; Basal: p=0.32). All images taken at 20×. Scale bar=50 μm. f) ELISA measurements of VEGF secretion under hypoxic conditions (6h) mimicking from reduced choroidal blood flow has been implicated in the pathophysiology of age related macular degeneration. (Mukherjee, P. K. et al. Photoreceptor outer segment phagocytosis attenuates oxidative stress-induced apoptosis with concomitant neuroprotectin D1 synthesis. Proc Natl Acad Sci USA 104, 13158-13163, (2007)) nd serves as a metabolic stressor to determine the susceptibility of L-ORD iPSC-RPE to hypoxia-driven EMT. Similar to normoxic conditions shown in FIG. 1i) L-ORD patient iPSC-RPE (N=10; Ap: 1.89±0.30; Ba: 1.8±0.24) secrete VEGF in a non-polarized manner compared to unaffected siblings (N=9; Ap: 0.78±0.16; Ba: 1.59±0.36) (Ap: p=0.005; Ba: p=0.63). Prior treatment (2 weeks) with metformin protects L-ORD patient RPE (N=6; Ap: 0.59±0.09; Ba: 1.8±0.24) against hypoxia-driven EMT and restores apical/basal VEGF polarity similar to untreated or metformin treated unaffected siblings (N=9; Ap: 0.98±0.16; Ba: 1.64±0.33) (Ap: p=0.09; Ba: p=0.64). g) The effect of POS feeding on the expression of dedifferentiation (EMT)-related genes in L-ORD patient iPSC-RPE compared to unaffected siblings. 7-days POS feeding (shown in white) causes an increased in the expression of EMT-related genes in L-ORD patients compared to unaffected siblings. Metformin treatment (shown in red) during the 7-days POS feeding suppresses the expression of EMT related genes. h) Table of results from retrospective clinical study reveals metformin delays age of onset of nonexudative age-related macular degeneration (362.51/H35.31). In patients ages 50-59, metformin delays the age of onset from 56 years of age (n=157, no metformin) to 58.5 years of age (n=16, with metformin) (p=0.001).

### Example 10—Novel Therapeutics to Improve and Maintain Retinal Pigment Epithelium (RPE) Healthy Phenotype in RPE Disorders

A siRNA screen was performed to identify candidate genes and pathways required to maintain epithelial phenotype of iPSC-RPE; using a reporter induced pluripotent stem (iPS) cell line that expresses GFP when differentiated into RPE. With this approach NOX4 was identified. NOX4 is a NADPH Oxidase whose inhibition strongly promotes epithelial phenotype in injured RPE cells

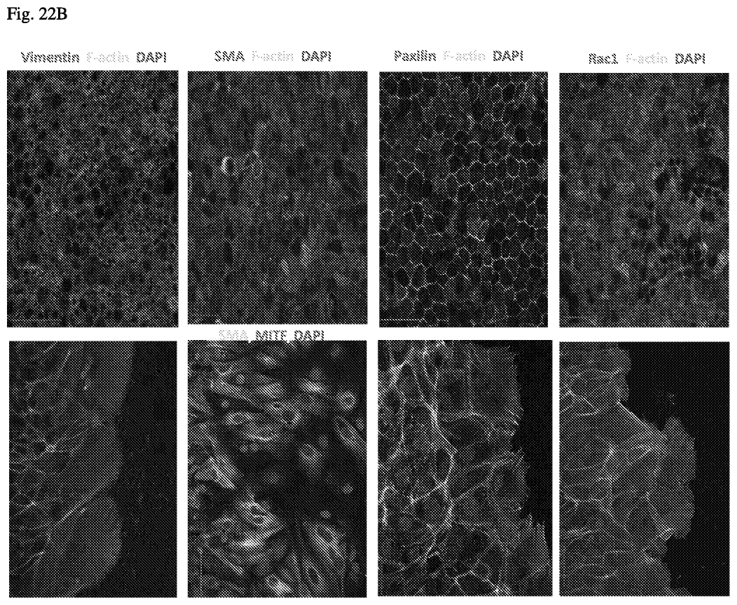

Retinal injuries induce RPE-EMT which is characterized by the dedifferentiation, proliferation, and migration of the RPE. FIGS. 22A and 22B showed that mechanical injury in the model is able to mimic the features of RPE-EMT in vivo; and after mechanical injury the RPE cells undergo to EMT showing the characteristic morphology and markers of EMT.

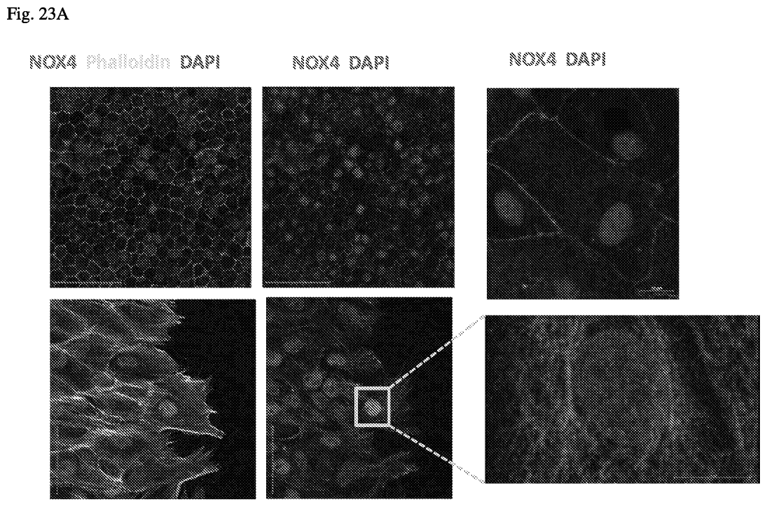

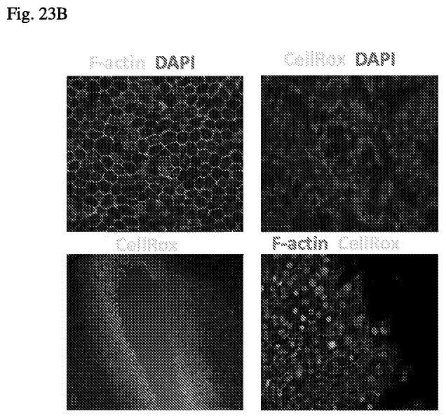

NOX4 is a NADPH enzyme and its primary role is to generate reactive oxygen species (ROS). NOX4 is highly expressed in the injured RPE. FIG. 23A show that Nox4 is present in the intact RPE, and highly expressed in the injured RPE. Also, in FIG. 23B, it is shown that injured RPE generates increased levels of ROS in comparison with intact RPE by using a nuclear dye that becomes fluorescent when oxidized.

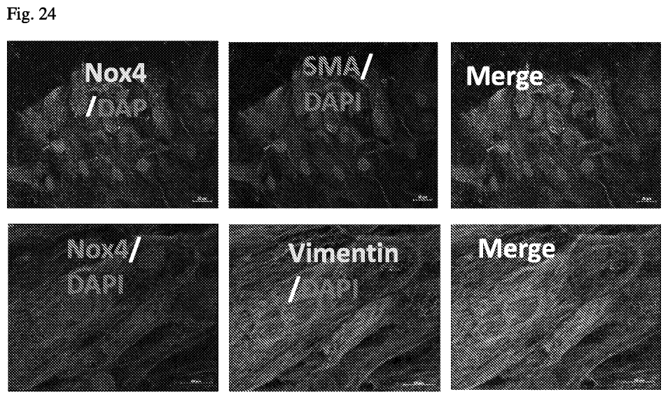

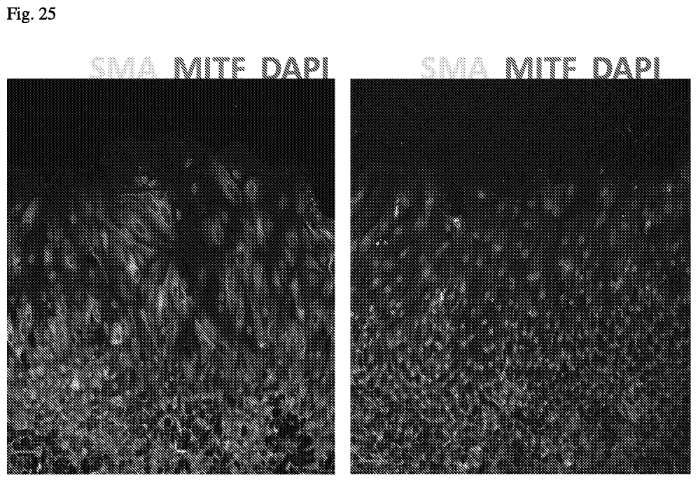

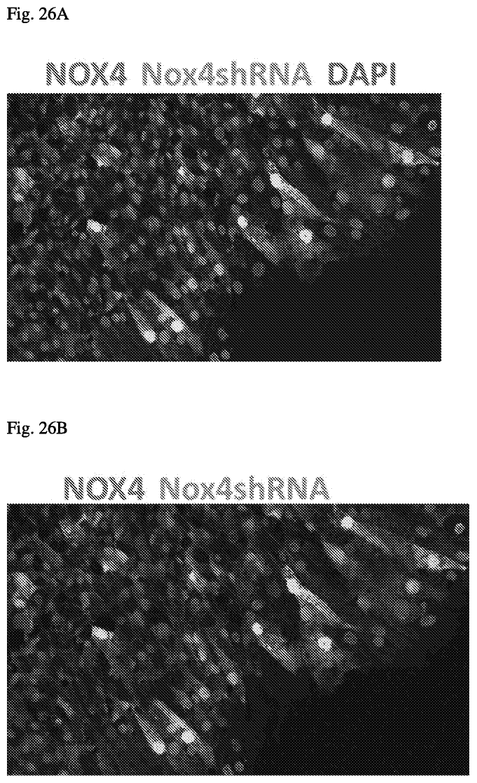

FIG. 24. shows that NOX4 colocalize with Cytoskeletal proteins that are known as a EMT markers, Vimentin and Smooth Muscle Actin (SMA), the association of NOX4 with EMT markers is an indication of the role of NOX4 in EMT.

FIG. 25. Shows that Pharmacological inhibition of NOX4 using VAS2870 Down-regulates SMA an EMT marker.

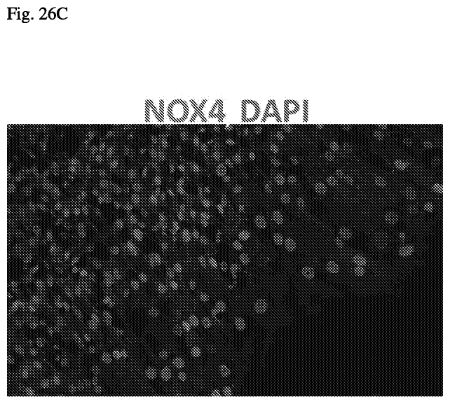

FIGS. 26A-26C show the knockdown of NOX4 by using shRNA and confirms the successful downregulation of NOX4.

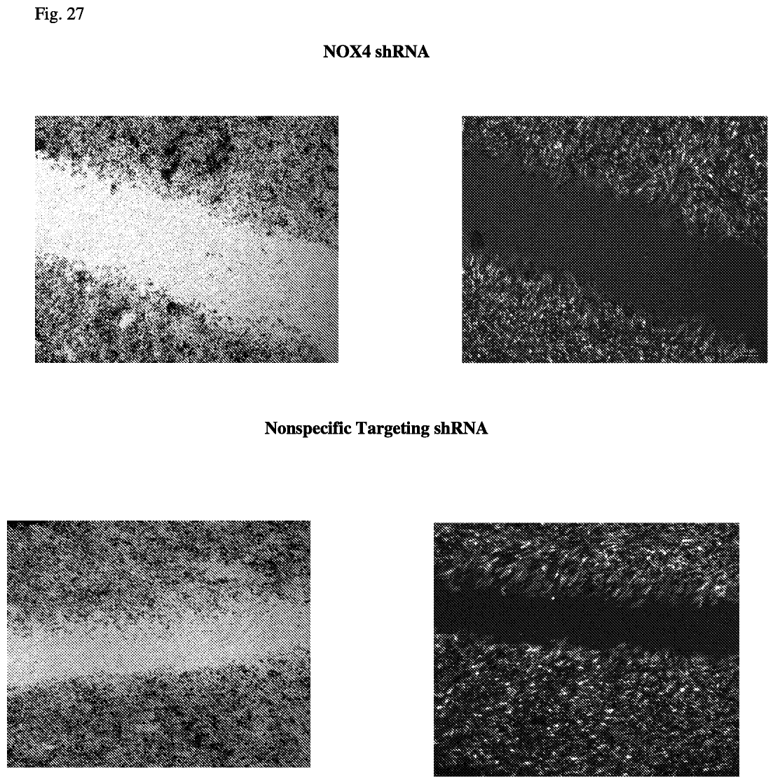

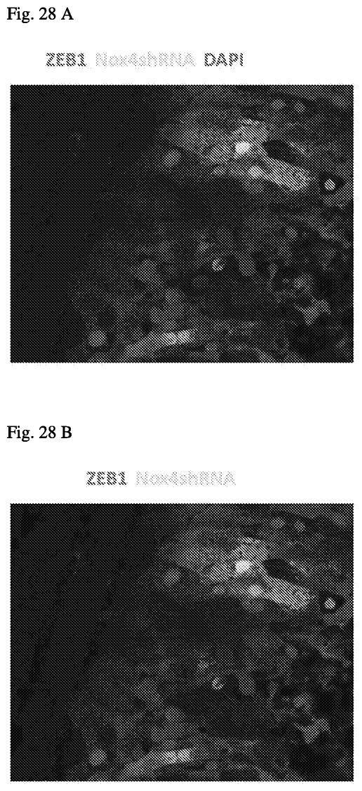

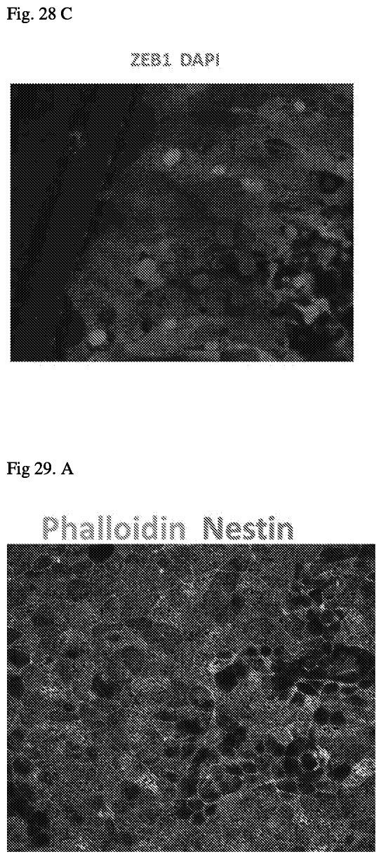

FIG. 27 shows down-regulation of NOX4 using shRNA decreased cell migration in injured RPE. The downregulation of NOX4 downregulates ZEB1—an EMT marker—as shown in FIGS. 28A-28C.

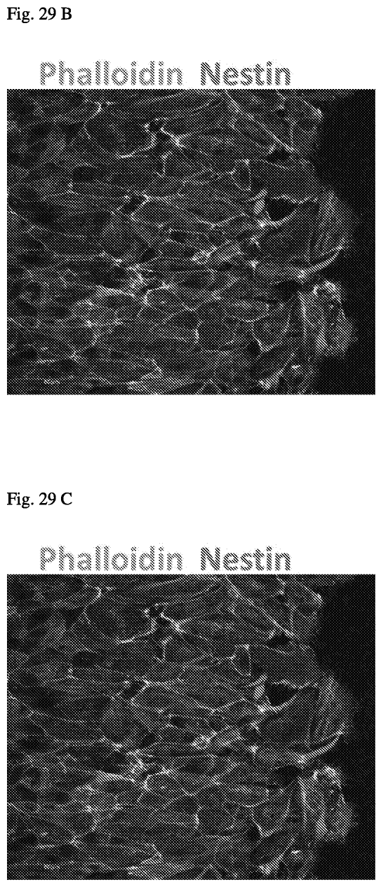

FIGS. 29A and 29B show that NOX4 shRNA lentiviral particles successfully downregulates Nestin in scratched RPE

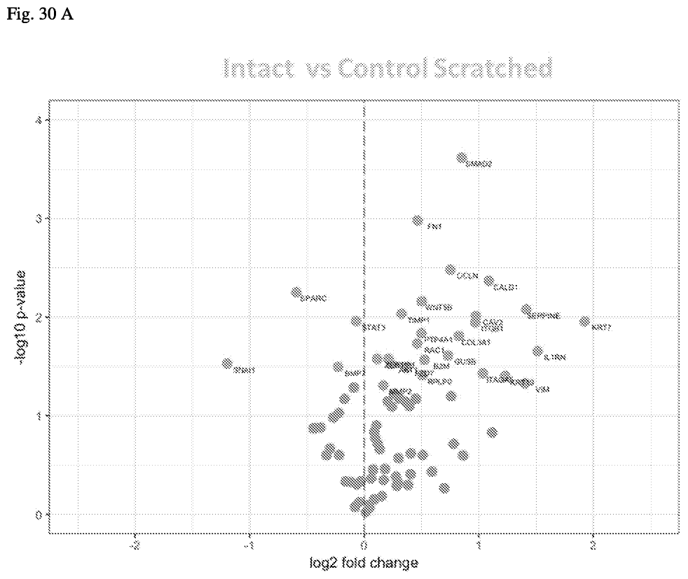

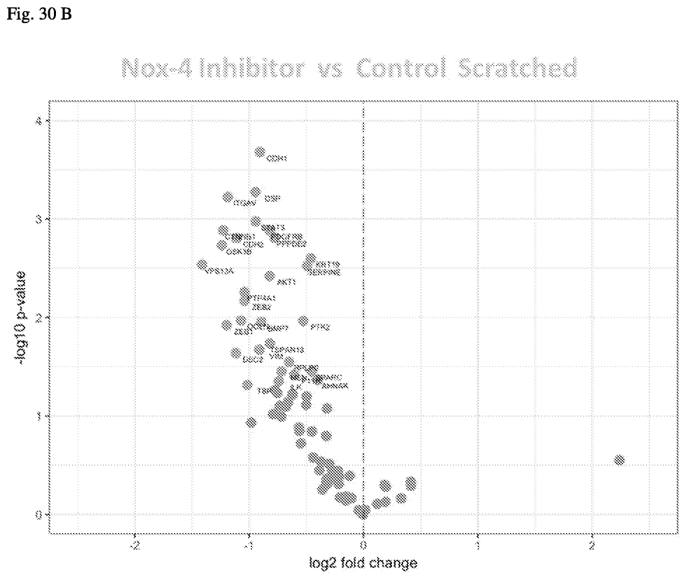

By performing pharmacological inhibition of NOX4 it was confirmed that inhibition of Nox4 effectively downregulates the expression of EMT markers as shown in FIGS. 30A and 30B.

As a result of this experiment, it has been shown that NOX4 is a novel target gene, whose expression modulates epithelial phenotype of human Retinal Pigment Epithelium (RPE). Pharmacological inhibitors that modulate the activity of NOX4 can be used as therapeutics to treat RPE disorders like proliferative viteroretinopathy (PVR), age-related and inherited retinal degenerations, and cancer.

### Example 11—Metformin Treatment Ameliorates Stargardt's Disease

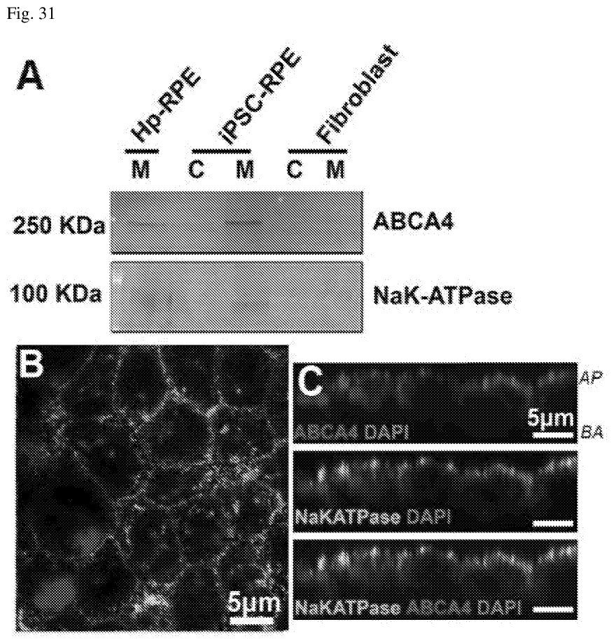

Stargardt disease is a rare inherited retinal degeneration, affecting ˜30,000 people in the U.S., with no current treatment. Progressive photoreceptor (PR) cell death induced by atrophied retinal pigment epithelium (RPE) leads to vision loss in patients. In its etiology, Stargardt is similar to AMD. Both diseases show sub and intra RPE deposits and RPE atrophy. But Stargadrt is a monogenic disease unlike AMD which is a polygenic disease. Stargardt is primarily caused by mutations in gene ABCA4, an ortholog of ABCA1—a known cholesterol transporter in the RPE Briggs, C. E., et al., Mutations in ABCR (ABCA4) in patients with Stargardt macular degeneration or cone-rod degeneration. Investigative ophthalmology & visual science, 2001. 42(10): p. 2229-2236; R Sparrrow, J., D. Hicks, and C. P Hamel, The retinal pigment epithelium in health and disease. Current molecular medicine, 2010. 10(9): p. 802-8231. Functional interactions between the RPE and PR cells are required for vision; thus, atrophied RPE rapidly leads to PR degeneration and blindness in many cases R Sparrrow, J., D. Hicks, and C. P Hamel, The retinal pigment epithelium in health and disease. Current molecular medicine, 2010. 10(9): p. 802-8231. RPE apical surface proteins are required for mediating RPE-PR functional interaction. Cell surface capturing technology (CSC) was used to selectively identify apical and basal surface proteome of polarized RPE monolayer. CSC helped identify several previously unreported proteins on the RPE membrane, including ABCA4, present predominantly on the apical side of RPE cells Khristov, V., et al., Polarized Human Retinal Pigment Epithelium Exhibits Distinct Surface Proteome on Apical and Basal Plasma Membranes, in The Surfaceome. 2018, Springer. p. 223-2471. ABCA4 expression on the RPE cell membrane was confirmed by Western blot (FIG. 31A) and its apical localization with immunostaining, as shown in (FIG. 31B, C). This result has challenged the current dogma that ABCA4 is exclusively expressed in PRs, and RPE atrophy seen in patients is solely due to RPE cells phagocytosing toxic material accumulated in POS due of ABCA4 mutation [Molday, R. S., M. Zhong, and F. Quazi, The role of the photoreceptor ABC transporter ABCA4 in lipid transport and Stargardt macular degeneration. Biochimica et Biophysica Acta (BBA)-Molecular and Cell Biology of Lipids, 2009; 1791(7): p. 573-583., Maugeri, A., et al., Mutations in the ABCA4 (ABCR) gene are the major cause of autosomal recessive cone-rod dystrophy. The American Journal of Human Genetics, 2000. 67(4): p. 960-966].

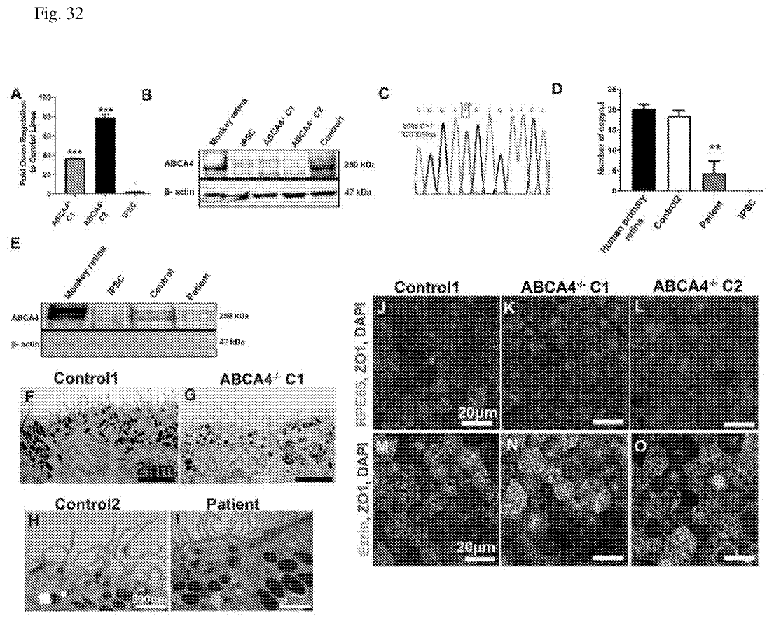

To elucidate the role of ABCA4 in RPE and disease pathogenesis, w Stargardt iPSC-derived RPE (iRPE) with complete loss of ABCA4 function was developed as an in vitro disease model (FIG. 32). We successfully generated two ABCA4−/− iPSC lines and derived fully mature RPE cells (ABCA4−/−C1 and ABCA4−/−C2; FIG. 32). ABCA4 knock out was confirmed by qRT PCR, ddPCR, and Western blot (FIG. 32A, B). In addition, a Stargardt patient iPSC line was derived and differentiated into mature RPE cells. Sanger sequencing confirmed the presence of the mutation (C>T in exon 44 at 6088 bp position) in patient-iRPE (FIG. 32C). ddPCR and Western blot analysis corroborated that mutation causes the non-sense mRNA decay of mutant ABCA4 mRNA in patient-iRPE (FIG. 32 D-E). In terms of molecular (FIG. 32 J-O), structural (FIG. 32 F-I), and functional validation patient and ABCA4−/− iRPE behaved similar to control iRPE monolayers (Control1—isogenic control for ABCA4−/−C1&C2, Control2-unaffected sibling for patient iRPE) (FIG. 32 F-O), suggesting there are no developmental defects in RPE due to ABCA4 loss of function.

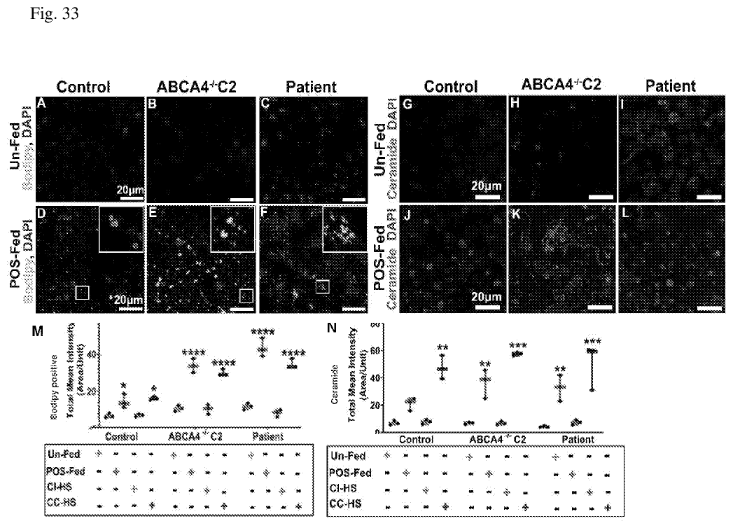

These results prompted the inventors to investigate the role of ABCA4 in mature iRPE monolayer. It was hypothesized that the ABCA4 mutation leads to cell autonomous functional defects in the RPE. To test this hypothesis, Stargardt iRPE (ABCA4−/− clones and patient) were treated with wild type POS for 6 days and the accumulation of intracellular and sub-RPE lipid deposits (one of the disease phenotypes) was evaluated. Analysis of intra/subcellular lipid deposits using BODIPY staining (lipid dye, BODIPY505/515) showed a 2-3 fold increase in lipid accumulation in wild type POS treated Stargardt RPE (compare FIG. 33 A-C with D-F, quantification in G), supporting the hypothesis of cell autonomous defects in Stargardt iRPE cells. To investigate whether these cell autonomous defects are enhanced by dysregulated complement signaling as the accumulation of toxic byproducts of the visual cycle (A2E and lipofuscin) are known to cause complement signaling induced inflammation, Stargardt iRPE was treated with activated human serum (CC-HS) or inactivated human serum (CI-HS) [Lenis, T. L., et al., Complement modulation in the retinal pigment epithelium rescues photoreceptor degeneration in a mouse model of Stargardt disease. Proceedings of the National Academy of Sciences, 2017. 114(15): p. 3987-3992]. Consistent with the previous report, 48-hour treatment of CC-HS triggered increased (2-3 fold) intra and sub-cellular lipid deposits in Stargardt iRPE vs. control cells (FIG. 33 G, compare CC-HS vs. CI-HS).

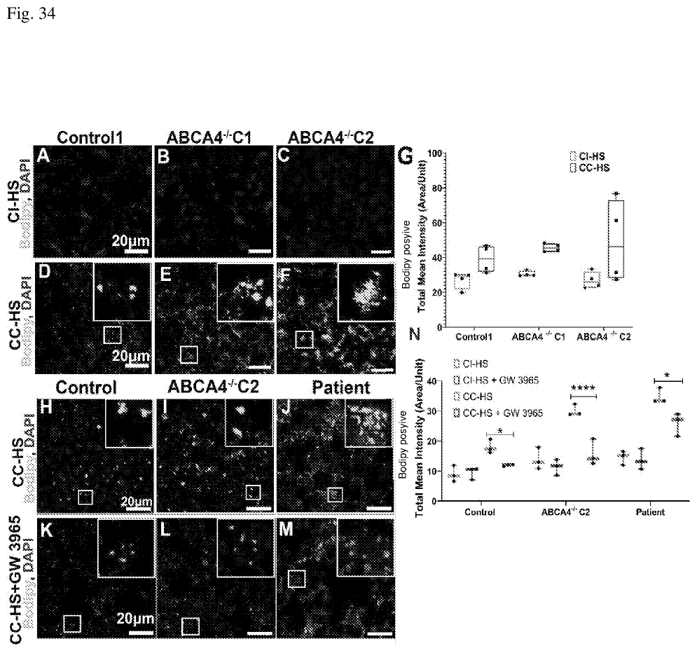

In the pigmented Abca4−/− mouse model, lipofuscin accumulation caused ceramide increase at the RPE's apical side, inducing early endosomes (EE) biogenesis and fusion, and increased C3a resulting activation of the mechanistic target of rapamycin (mTOR), a master regulator of autophagy [Kaur, G., et al., Aberrant early endosome biogenesis mediates complement activation in the retinal pigment epithelium in models of macular degeneration. Proceedings of the National Academy of Sciences, 2018. 115(36): p. 9014-9019]. A 4-5 fold increase in apical ceramide accumulation in Stargardt iRPE was observed when exposed to POS regimen and CC-HS treatment, as seen in the Abca4−/− mouse model. Overall, this work showed that ABCA4 KO and patient iRPE cells represent a physiologically relevant in vitro disease model for Stargardt disease and that ABCA4 loss of function triggers a cell autonomous disease phenotype in RPE cells. To further understand the ABCA4 driven mechanism in disease pathogenesis, this example focuses on ABCA1- an ABCA4 homolog, involved in cholesterol transport. The 64.5% amino acid homology between two proteins suggests that ABCA4 might also be involved in cholesterol and lipid hemostasis [Quazi, F. and R. S. Molday, Differential phospholipid substrates and directional transport by ATP-binding cassette proteins ABCA1, ABCA7, and ABCA4 and disease-causing mutants. Journal of Biological Chemistry, 2013. 288(48): p. 34414-34426, Tanaka, A. R., et al., Human ABCA1 contains a large amino-terminal extracellular domain homologous to an epitope of Sjögren's Syndrome. Biochemical and biophysical research communications, 2001. 283(5): p. 1019-1025, Storti, F., et al., Impaired ABCA1/ABCG1-mediated lipid efflux in the mouse retinal pigment epithelium (RPE) leads to retinal degeneration. Elife, 2019. 8: p. e45100.]. To determine if both ABCA proteins work through the lipid handling pathway, the effects of modulating ABCA1 expression on changes the disease phenotype in Stargardt RPE cells was observed. An shRNA knockdown of ABCA1 in ABCA4 iRPE cells was performed and the cells were treated with CC-HS. The ABCA1 KD exacerbated the lipid deposits in ABCA4 RPE cells, as seen by the BODIPY stain. (FIG. 34A-G), In contrast, lipid accumulation defects in Stargardt RPE cells were rescued by ABCA1 overactivation using GW3965 (ABCA1 activator) (FIG. 34 H-N).

Lipofuscin, a yellowish lipid-rich deposit likely formed from undigested cellular lipid and visual cycle metabolites, is a characteristic feature of Stargardt patient eyes. Lipofuscin accumulation has been associated with RPE dysfunction and its atrophy [Sparrow, J. R., et al., A2E, a fluorophore of RPE lipofuscin: can it cause RPE degeneration?, in Retinal Degenerations. 2003, Springer. p. 205-211; Sparrow, J. R. and M. Boulton, RPE lipofuscin and its role in retinal pathobiology. Experimental eye research, 2005. 80(5): p. 595-606]. These results led to the hypothesis if a drug decreases the rate of lipofuscin accumulation or increases lipofuscin clearance in the RPE—it could possibly delay RPE and retina degeneration associated with this disease [Issa, P. C., et al., Rescue of the Stargardt phenotype in Abca4 knockout mice through inhibition of vitamin A dimerization. Proceedings of the National Academy of Sciences, 2015. 112(27): p. 8415-8420; Tanna, P., et al., Stargardt disease: clinical features, molecular genetics, animal models and therapeutic options. British Journal of Ophthalmology, 2017. 101(1): p. 25-30]. Based on the collected data, it was hypothesized that a lipid-lowering drug-metformin hydrochloride could act as a potential therapeutic intervention for ABCA4 patients. Metformin is a clinically approved medication for type 2 diabetes that enhances cellular lipid metabolism by activating the AMPK pathway and increase lysosomal activity by decreasing lysosomal pH via endosomal Na+/H+ exchangers, and the V-ATPase [Zhang, C.-S., et al., Metformin activates AMPK through the lysosomal pathway. Cell metabolism, 2016. 24(4): p. 521-522; Feng, Y., et al., Metformin promotes autophagy and apoptosis in esophageal squamous cell carcinoma by downregulating Stat3 signaling. Cell death & disease, 2014. 5(2): p. e1088-e1088; Wang, N., et al., Metformin improves lipid metabolism disorders through reducing the expression of microsomal triglyceride transfer protein in OLETF rats. Diabetes research and clinical practice, 2016. 122: p. 170-178; Wang, Y., et al., Metformin induces autophagy and G0/G1 phase cell cycle arrest in myeloma by targeting the AMPK/mTORC1 and mTORC2 pathways. Journal of Experimental & Clinical Cancer Research, 2018. 37(1): p. 63; Anurag, P. and C. Anuradha, Metformin improves lipid metabolism and attenuates lipid peroxidation in high fructose fed rats. Diabetes, Obesity and Metabolism, 2002. 4(1): p. 36-42; Kim, J and Y. J. You, Regulation of organelle function by metformin. IUBMB life, 2017. 69(7): p. 459-469.]. These mechanisms of actions are predicted to enable Stargardt RPE cells manage lipofuscin clearance more efficiently. A clinical trial was proposed to test the ability of metformin to ameliorate disease phenotype in Stargardt patients and to discover its mechanism of action in Stargardt-mouse and the iRPE model. The significance of this approach relies on a previously underappreciated role of ABCA4 in RPE cells. It is noteworthy that the ability to recapitulate Stargardt disease phenotype in ABCA4 mutant iRPE without the use of Stargardt POS suggests a cell-autonomous lipid metabolism defect in these cells. Loss of this cell autonomous role of ABCA4 in RPE lipid metabolism contributes to Stargardt disease pathology and improvement of the activity of this pathway may change disease course.

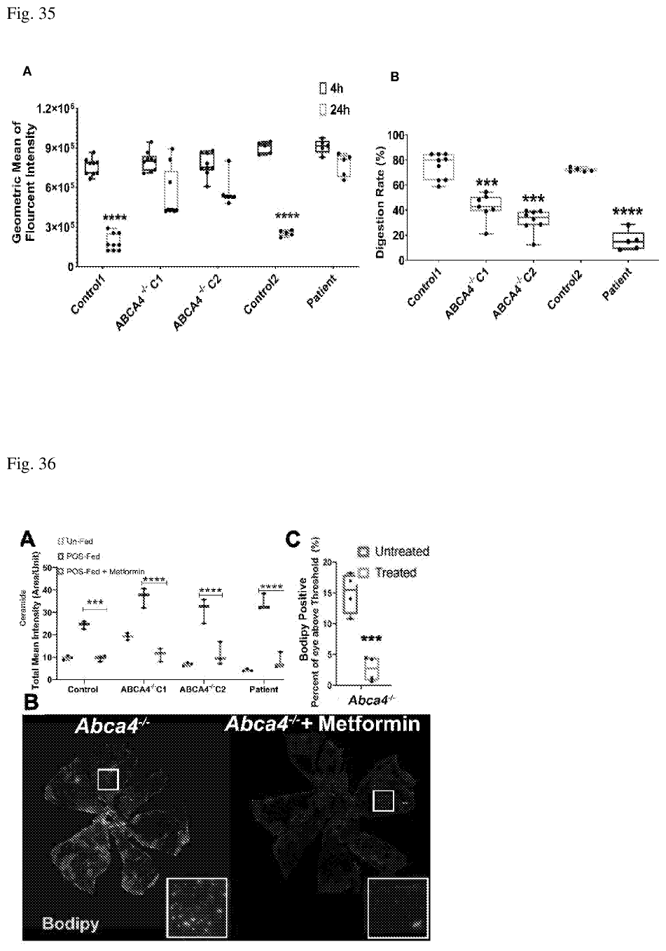

POS digestion defect in Stargardt iRPE cells. Increased lipid and ceramide accumulation in Stargardt iRPE cells suggested a potential lysosomal defect and reduced ability to digest POS that may lead to RPE atrophy and trigger photoreceptor degeneration over time [Carr, A.-J., et al., Molecular characterization and functional analysis of phagocytosis by human embryonic stem cell-derived RPE cells using a novel human retinal assay. Molecular vision, 2009. 15: p. 283.]. To determine if Stargardt iRPE is defective in POS digestion, ABCA4 mutant and control iRPE cells were fed with pHRhodo dye (fluoresces only inside lysosomes) labeled wild type POS (10/RPE cell) for 4 hrs. The dye label helped distinguish between POS uptake (measured after 4 hrs of POS feeding) and the digestion rates (measured after 24 hrs of POS feeding). Stargardt iRPE cells showed a similar ability to uptake POS as control cells (4h time point) (FIG. 35A). However, a 50-70% reduced digestion rate (24h time point) was observed in Stargardt iRPE (FIG. 35B), as compared to control cells. These results suggest that ABCA4 mutation in iRPE cells causes disrupted endo-lysosomal dysfunction, likely contributing to defective lipid metabolism and cellular dysfunction.

Metformin treatment ameliorates lipid deposits in Stargardt iRPE, Reduced ability of Stargardt iRPE cells to digest wild type POS suggested that endo-lysosomal dysfunction is at the center of lipid homeostasis defect in diseased cells. Metformin improves lysosomal function and lipid metabolism [Wang, N., et al., Metformin improves lipid metabolism disorders through reducing the expression of microsomal triglyceride transfer protein in OLETF rats. Diabetes research and clinical practice, 2016. 122: p. 170-178., Anurag, P. and C. Anuradha, Metformin improves lipid metabolism and attenuates lipid peroxidation in high fructose-fed rats. Diabetes, Obesity and Metabolism, 2002. 4(1): p. 36-42; Kim, J. and Y. J. You, Regulation of organelle function by metformin. IUBMB life, 2017. 69(7): p. 459-469]. It was hypothesized that metformin treatment will improve lysosomal activity and lipid metabolism in Stargardt iRPE cells, thus reducing ceramide and lipid accumulation to ameliorate disease phenotypes. To evaluate the therapeutic effect of metformin in an in vitro system, Stargardt and healthy cells were treated with wild type POS (10 POS/cell) for 6 consecutive days in either RPE media+ vehicle or in RPE media containing 3 mM metformin As compared to vehicle treated Stargardt iRPE, metformin treatment significantly reduced (3-4 fold) ceramide levels in POS fed Stargardt iRPE. (FIG. 36A). To translate metformin as a potential treatment of Stargardt disease, it was tested in the Abca4−/− mouse model that recapitulates phenotypes of Stargardt retinopathy, including ceramide and lipid-rich sub-RPE deposits Maur, G., et al., Aberrant early endosome biogenesis mediates complement activation in the retinal pigment epithelium in models of macular degeneration. Proceedings of the National Academy of Sciences, 2018. 115(36): p. 9014-9019., Issa, P. C., et al., Fundus autofluorescence in the Abca4−/− mouse model of Stargardt disease—correlation with accumulation of A2E, retinal function, and histology. Investigative ophthalmology & visual science, 2013. 54(8): p. 5602-5612.1. Mice received oral metformin doses at 400 or 800 mg/day for three months—comparable to the human dose. These doses do not lead to hypoglycemia in treated mice. Mass-spectrometry analysis of the eyes collected from treated animals showed a comparable amount of metformin iRPE/choroid, retina, and plasma, suggesting that drug reaches to the target tissue (data not shown). Our data from RPE/choroid flat-mount of treated Abca4−/− mice showed that metformin treatment drastically reduced lipid levels in the Abca4−/− mice (FIG. 36 B-C). These results confirmed the hypothesis of metformin as a potential treatment of Stargardt and AMD patients.

### Example 12—Intravitreous Injection, Sub-Tenon Injection, Sub-Retinal Injection, and Topical Ocular Treatment Methods Ameliorate AMD and Stargardt's Disease

To confirm efficacy of treatment, the treatments of Examples 1-11 are repeated using a variety of administration methods. In particular, the treatments of Examples 1-11 are repeated using intravitreous injection, sub-tenon injection, sub-retinal injection and topical ocular administration methods. The results of these additional administrations demonstrates the efficacy of treatment using these administration methods.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1-52. (canceled)

53. A method of treating a retinal disease comprising administering to a patient in need thereof a pharmaceutically effective amount of Metformin or a pharmaceutically acceptable salt thereof.

54. The method of treating a retinal disease of claim 53, wherein the retinal disease is macular or peripheral retinal degeneration, geographic atrophy, choroidal neovascularization, retinal pigment epithelium atrophy, macular dystrophy, Stargardt's disease, a Stargardt's-like disease, Best disease, vitelliform macular dystrophy, adult vitelliform dystrophy, retinitis pigmentosa, proliferative vitreoretinopathy, retinal detachment, pathologic myopia, diabetic retinopathy, CMV retinitis, occlusive retinal vascular disease, retinopathy of prematurity (ROP), choroidal rupture, ocular histoplasmosis syndrome (POHS), toxoplasmosis, or Leber's congenital amaurosis.

55. The method of claim 54, wherein the retinal disease is macular retinal degeneration.

56. The method of claim 53, wherein the Metformin or a pharmaceutically acceptable salt thereof is administered in the form of a pharmaceutical composition wherein the pharmaceutical composition comprises Metformin or a pharmaceutically acceptable salt thereof and one or more pharmaceutically acceptable carriers.

57. The method of claim 53, wherein the Metformin or a pharmaceutically acceptable salt thereof is administered topically to the eye of the patient.

58. The method of claim 53, wherein the Metformin or a pharmaceutically acceptable salt thereof is administered to the patient through intravitreous injection, sub-tenon injection, or sub-retinal injection.

59. The method of claim 56, wherein the composition is administered topically to the eye of the patient.

60. The method of claim 56, wherein the composition is administered to the patient through intravitreous injection, sub-tenon injection, or sub-retinal injection.

61. A method of restoring retinal pigment epithelium cells degeneration comprising administering to a patient in need thereof a pharmaceutically effective amount of Metformin or a pharmaceutically acceptable salt thereof.

62. The method of claim 61, wherein the Metformin or a pharmaceutically acceptable salt thereof is administered in the form of a pharmaceutical composition wherein the pharmaceutical composition comprises Metformin or a pharmaceutically acceptable salt thereof and one or more pharmaceutically acceptable carriers.

63. The method of claim 61, wherein the Metformin or a pharmaceutically acceptable salt thereof is administered topically to the eye of the patient.

64. The method of claim 61, wherein the Metformin or a pharmaceutically acceptable salt thereof is administered to the patient through intravitreous injection, sub-tenon injection, or sub-retinal injection.

65. The method of claim 62, wherein the composition is administered topically to the eye of the patient.

66. The method of claim 62, wherein the composition is administered to the subject through intravitreous injection, sub-tenon injection, or sub-retinal patient.

67. A method of treating Stargardt's disease or a Stargardt's-like disease comprising administering to a patient in need thereof Metformin or a pharmaceutically acceptable salt thereof.

68. The method of claim 67, wherein the Metformin or a pharmaceutically acceptable salt thereof is administered in the form of a pharmaceutical composition wherein the pharmaceutical composition comprises Metformin or a pharmaceutically acceptable salt thereof and one or more pharmaceutically acceptable carriers.

69. The method of claim 67, wherein the Metformin or a pharmaceutically acceptable salt thereof is administered topically to the eye of the patient.

70. The method of claim 67, wherein the Metformin or a pharmaceutically acceptable salt thereof is administered to the patient through intravitreous injection, sub-tenon injection, or sub-retinal injection.

71. The method of claim 68, wherein the composition is administered topically to the eye of the patient.

72. The method of claim 68, wherein the composition is administered to the patient through intravitreous injection, sub-tenon injection, or sub-retinal injection.
